SEQUENCE LISTING (1 sequence)

SEQ ID NO: 1 - protein, 43 aa, Homo sapiens
DAEFRHDSGYEVHHQKLVFFAEDVGSNKGAIIGLMVGGVVIAT

(19) United States
(12) Patent Application Publication
(10) Pub. No.: US 2015/0140672 A1
(43) Pub. Date: May 21, 2015

(54) METHODS FOR DETECTING AMYLOID BETA AMYLOIDOSIS

(71) Applicant: WASHINGTON UNIVERSITY, ST. LOUIS, MO (US)

(72) Inventors: Randall Bateman, St. Louis, MO (US); Kwasi Mawuenyega, St. Louis, MO (US); Vitaliy Ovod, St. Louis, MO (US); Anna Bareiss, St. Louis, MO (US); Tom Kasten, St. Louis, MO (US); David Holtzman, St. Louis, MO (US)

(21) Appl. No.: 14/523,148

(22) Filed: Oct. 24, 2014

Related U.S. Application Data
(63) Continuation-in-part of application No. 14/366,831, filed on Jun. 19, 2014.
(60) Provisional application No. 61/577,439, filed on Dec. 19, 2011.
(60) Provisional application No. 61/895,601, filed on Oct. 25, 2013.

(51) Int. Cl. G01N 33/68

(52) U.S. Cl. CPC G01N 33/6893; G01N 2333/4709; G01N 2800/7047; G01N 2560/00; G01N 2458/15
USPC 436/86

(57) ABSTRACT

The present invention relates to methods of detecting, diagnosing, monitoring, and assessing treatment effects for Aβ amyloidosis, early in the course of clinical disease or prior to the onset of brain damage and clinical symptoms.

### CROSS REFERENCES TO RELATED APPLICATIONS

This application claims the priority of U.S. provisional application No. 61/895,601, filed Oct. 25, 2013, and is a continuation-in-part of U.S. application Ser. No. 14/366,831 filed Jun. 19, 2014, which is a national stage entry of PCT/US12/70623, filed Dec. 19, 2012, which claims the priority of U.S. provisional application No. 61/577,439 filed Dec. 19, 2011, each of which is hereby incorporated by reference in its entirety.

### GOVERNMENTAL RIGHTS

This invention was made with government support under R01 NS065667 awarded by NINDS. The government has certain rights in the invention.

### FIELD OF THE INVENTION

The present invention relates to methods of detecting, diagnosing, monitoring, and assessing treatment effects for Aβ amyloidosis, early in the course of clinical disease or prior to the onset of brain damage and clinical symptoms.

### BACKGROUND OF INVENTION

Aβ amyloidosis is clinically defined as evidence of Aβ deposition in the brain either by amyloid imaging (e.g. PiB PET) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Subjects with Aβ amyloidosis are at an increased risk of developing a disease associated with Aβ amyloidosis. Diseases associated with Aβ amyloidosis include, but are not limited to, Alzheimer's Disease (AD), cerebral amyloid angiopathy, Lewy body dementia, and inclusion body myositis.

Alzheimer's Disease (AD) is the most common cause of dementia and is an increasing public health problem. It is currently estimated to afflict 5 million people in the United States, with an expected increase to 13 million by the year 2050 (Herbert et al 2001, Alzheimer Dis. Assoc. Disord. 15(4): 169-173). AD, like other central nervous system (CNS) degenerative diseases, is characterized by disturbances in protein production, accumulation, and clearance. In AD, dysregulation in the metabolism of the protein, amyloid-beta (Aβ), is indicated by a massive buildup, or Aβ amyloidosis, of this protein in the brains of those with the disease. AD leads to loss of memory, cognitive function, and ultimately independence. It takes a heavy personal and financial toll on the patient and the family. Because of the severity and increasing prevalence of this disease in the population, it is urgent that better treatments be developed. Currently, there are some medications that modify symptoms of AD, however, there are no disease-modifying treatments. Disease-modifying treatments will likely be most effective when given before the onset of permanent brain damage. However, by the time clinical diagnosis of AD is made, extensive neuronal loss has already occurred (Price et al. 2001, Arch. Neurol. 58(9): 1395-1402).

Amyloid imaging (e.g. PiB PET) and/or cerebrospinal fluid (CSF) Aβ42 or Aβ42/40 ratio are being used in limited settings to attempt to identify individuals at risk of developing AD before the onset of clinical symptoms or of effectively measuring the effects of treatments that may prevent the onset or slow the progression of the disease. Both of these methods measure static amounts of amyloid depositions and, therefore, have limitations. A need exists, therefore, for improved methods to detect and monitor Aβ amyloidosis.

### SUMMARY OF INVENTION

One aspect of the present disclosure encompasses a method for detecting Aβ amyloidosis in a subject, the method comprising measuring the in vivo relative labeling of at least two Aβ variants in a blood sample from the subject, and calculating the ratio of relative labeling of the first Aβ variant to the relative labeling of the second Aβ variant, wherein a ratio other than 1 indicates the presence of Aβ amyloidosis. The in vivo relative labeling of Aβ42 and another Aβ variant may be determined as described in Section I(a). A blood sample may be obtained from the subject at any point along the labeling curve including, but not limited to, at about 1 min to about 4 hours, about 1 min to about 8 hours, about 1 min to about 12 hours, about 2 hours to about 8 hours, about 8 hours to about 24 hours, about 8 hours to about 36 hours, or about 12 hours to about 36 hours.

In another aspect, the present disclosure encompasses a method for detecting Aβ amyloidosis in a subject, the method comprising measuring the in vivo relative labeling of the in vivo relative labeling of Aβ42 and another Aβ variant in a blood sample from the subject, and calculating the ratio of relative labeling of the Aβ42 to the relative labeling of the other Aβ variant, wherein a ratio other than 1 indicates the presence of Aβ amyloidosis. The in vivo relative labeling of Aβ42 and another Aβ variant may be determined as described in Section I(a). In preferred embodiments, the other Aβ variant is selected from the group consisting of Aβ40, Aβ38 or total Aβ. A blood sample may be obtained from the subject at any point along the labeling curve including, but not limited to, at about 1 min to about 4 hours, about 1 min to about 8 hours, about 1 min to about 12 hours, about 2 hours to about 8 hours, about 8 hours to about 24 hours, about 8 hours to about 36 hours, or about 12 hours to about 36 hours.

In another aspect, the present disclosure encompasses a method for detecting Aβ amyloidosis in a subject, the method comprising measuring the in vivo relative labeling of Aβ42 and another Aβ variant in 1, 2, 3, 4, 5, or more biological samples obtained from the subject, and calculating the ratio of the Aβ42 Fractional Turnover Rate (FTR) to the FTR of the other Aβ variant, wherein a ratio of greater than about 1 indicates the presence of Aβ amyloidosis. FTR is the rate of irreversible loss of an Aβ variant from the central nervous system, and may be expressed as the sum of losses to CSF and other loss pathways in a compartmental-based model of Aβ turnover kinetics. The in vivo relative labeling of Aβ42 and another Aβ variant may be determined as described in Section I(a). In preferred embodiments, a biological sample is a blood sample or a CSF sample, and the other Aβ variant is selected from the group consisting of Aβ40, Aβ38 or total Aβ. One or more biological samples may be obtained from the subject at any point along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

In another aspect, the present disclosure encompasses a method for detecting Aβ amyloidosis in a subject, the method comprising measuring the in vivo relative labeling of Aβ42 and another Aβ variant in at least two (e.g. 2, 3, 4, 5 or more) biological samples obtained from the subject; determining the peak time for labeled Aβ42 and the other labeled Aβ variant; and calculating the ratio of the Aβ42 peak time to the peak time of the other Aβ variant; wherein a ratio of less than about 1 indicates the presence of Aβ amyloidosis. The in vivo relative labeling of Aβ42 and another Aβ variant may be determined as described in Section I(a). In preferred embodiments, a biological sample is a blood sample or a CSF sample, and the other Aβ variant is selected from the group consisting of Aβ40, Aβ38 or total Aβ. The two or more biological samples generally include at least one sample obtained during Aβ labeling increase and at least one sample obtained during Aβ labeling decrease. The biological samples may be obtained from the subject at points along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

In another aspect, the present disclosure encompasses a method for detecting Aβ amyloidosis in a subject, the method comprising measuring the in vivo relative labeling of Aβ42 in 1, 2, 3, 4, 5, or more biological samples obtained from the subject; and calculating the ratio of the Aβ42 exchange rate; wherein an Aβ42 exchange rate of greater than about 1 indicates the presence of Aβ amyloidosis. Aβ42 exchange rate refers to the rate of entry of an Aβ42 into an exchange compartment in a compartmental-model of Aβ turnover kinetics. The in vivo relative labeling of Aβ42 may be determined as described in Section I(a). In preferred embodiments, a biological sample is a blood sample or a CSF sample. The one or more biological samples may be obtained from the subject at any point along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

In another aspect, the present disclosure encompasses a method for monitoring the effectiveness of a therapeutic and/or treatment regimen, the method comprising measuring the in vivo relative labeling of Aβ42 and another Aβ variant in 1, 2, 3, 4, 5, or more biological samples obtained from the subject; and calculating the ratio of the Aβ42 Fractional Turnover Rate (FTR) to the FTR of the other Aβ variant before and during and/or after treatment, wherein a decrease in the value of the kinetic parameter, by an acceptable degree of statistical significance, indicates the treatment is effective, and no change or an increase in the kinetic parameter indicates the treatment is not effective. FTR is the rate of irreversible loss of an Aβ variant from the central nervous system, and may be expressed as the sum of losses to CSF and other loss pathways in a compartmental-based model of Aβ turnover kinetics. The in vivo relative labeling of Aβ42 and another Aβ variant may be determined as described in Section I(a). In preferred embodiments, a biological sample is a blood sample or a CSF sample, and the other Aβ variant is selected from the group consisting of Aβ40, Aβ38 or total Aβ. The one or more biological samples may be obtained from the subject at any point along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

In another aspect, the present disclosure encompasses a method for monitoring the effectiveness of a therapeutic and/or treatment regimen, the method comprising measuring the in vivo relative labeling of Aβ42 in 1, 2, 3, 4, 5, or more biological samples obtained from the subject; and calculating the Aβ42 FTR and/or the Aβ42 exchange rate before and during and/or after treatment, wherein a decrease in the value of the kinetic parameter, by an acceptable degree of statistical significance, indicates the treatment is effective, and no change or an increase in the kinetic parameter indicates the treatment is not effective. Aβ42 FTR is the rate of irreversible loss of Aβ42 from the central nervous system, and may be expressed as the sum of losses to CSF and other loss pathways in a compartmental-based model of Aβ turnover kinetics. Aβ42 exchange rate refers to the rate of entry of an Aβ42 into an exchange compartment in a compartmental-model of Aβ turnover kinetics. The in vivo relative labeling of Aβ42 may be determined as described in Section I(a). In preferred embodiments, a biological sample is a blood sample or a CSF sample. The one or more biological samples may be obtained from the subject at any point along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

In another aspect, the present disclosure encompasses a method for monitoring the effectiveness of a therapeutic and/or treatment regimen, the method comprising measuring the in vivo relative labeling of Aβ42 and another Aβ variant in at least two biological samples obtained from the subject; determining the peak time for labeled Aβ42 and the other labeled Aβ variant; and calculating the ratio of the Aβ42 peak time to the peak time of the other Aβ variant before and during and/or after treatment, wherein a decrease in the value of the kinetic parameter, by an acceptable degree of statistical significance, indicates the treatment is effective, and no change or an increase in the kinetic parameter indicates the treatment is not effective. The in vivo relative labeling of Aβ42 may be determined as described in Section I(a). In preferred embodiments, a biological sample is a blood sample or a CSF sample. The two or more biological samples generally include at least one sample obtained during Aβ labeling increase and at least one sample obtained during Aβ labeling decrease. The biological samples may be obtained from the subject at points along the labeling curve including, but not limited to, at about 1 min to about 4 hours, about 30 min to about 12 hours, about 8 hours to about 24 hours, about 8 hours to about 36 hours, about 12 hours to about 36 hours, or a combination thereof (0 hours=administration of a labeled moiety).

Other aspects and iterations of the invention are described more thoroughly below.

### BRIEF DESCRIPTION OF THE DRAWINGS

The application file contains at least one drawing executed in color. Copies of this patent application publication with color drawing(s) will be provided by the Office upon request and payment of the necessary fee.

FIG. 1 depicts a graph showing the percent ¹³C₆-leucine labeled TTR (orange circle and blue diamond) and Aβ42 (green hyphen and purple X) in blood over 12 hours for amyloid negative (solid line) and amyloid positive (dashed line) participants. Participants received an IV bolus of 800 mg labeled leucine. Note the increased labeling in participants with amyloidosis.

FIG. 2 depicts a graph showing the percent ¹³C₆-leucine labeled Aβ42/40 ratios (red hyphen and green triangles) and Aβ42/38 ratios (blue tick and red circle) in blood over 12 hours for amyloid negative (solid line) and amyloid positive (dashed line) participants. Participants received an IV bolus of 800 mg labeled leucine. Note the higher Aβ42/40 ratios in early hours (e.g. 1-3 hours) in amyloid positive versus amyloid negative participants.

FIG. 3 depicts a graph showing ¹³C₆-leucine labeling is higher in CDR>0 (dashed lines) versus CDR0 (solid lines) participants. Graphed separately is the label (y-axis) of TTR, Aβ42, Aβ40, Aβ38 and total Aβ over 12 hours (x-axis). Participants received an IV bolus of 800 mg labeled leucine.

FIGS. 4A and B depicts graphs showing the relative labeling of a first amyloid beta variant to a second amyloid beta (Aβ) variant by amyloid status 1.5 hours after administration of an IV bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIGS. 5A and B depicts graphs showing the relative labeling of a first amyloid beta variant to a second amyloid beta (Aβ) variant by amyloid status 1.5 hours after administration of ⁶C₁₃-leucine, combining data from subjects receiving either an IV bolus or an oral bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIGS. 6A and B depicts graphs showing the relative labeling of a first amyloid beta variant to a second amyloid beta (Aβ) variant by amyloid status 3 hours after administration an IV bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIGS. 7A and B depicts graphs showing the relative labeling of a first amyloid beta variant to a second amyloid beta (Aβ) variant by amyloid status 3 hours after administration of ⁶C₁₃-leucine, combining data from subjects receiving either an IV bolus or oral bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (B, top row), Aβ40 (A, bottom row), Aβ42 (B, top row), total Aβ (A, bottom row). TTR ratio=tracer-to-tracee ratio, also described as ratio of labeled to unlabeled amyloid beta. Amyloid status: amyloid negative=blue, amyloid positive=red.

FIGS. 8A and B depicts graphs showing the relative labeling of a first amyloid beta variant to a second amyloid beta (Aβ) variant by amyloid status 9 hours after administration of an IV bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (]B, bottom row). TTR ratio=tracer-to-tracee ratio, also described as ratio of labeled to unlabeled amyloid beta. Amyloid status: amyloid negative=blue, amyloid positive=red.

FIGS. 9A and B depicts graphs showing the relative labeling of a first amyloid beta variant to a second amyloid beta (Aβ) variant by amyloid status 9 hours after administration of ⁶C₁₃-leucine, combining data from subjects receiving either an IV bolus or oral bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIGS. 10A and B depicts graphs showing the relative labeling of a first amyloid beta variant to a second amyloid beta (Aβ) variant by amyloid status 24 hours after administration of an IV bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIGS. 11A and B depicts graphs showing the relative labeling of a first amyloid beta variant to a second amyloid beta (Aβ) variant by amyloid status 24 hours after administration of ⁶C₁₃-leucine, combining data from subjects receiving either an IV bolus or oral bolus of ⁶C₁₃-leucine. The first amyloid beta variant is identified on the top of each column, and from left to right reads: Aβ38, Aβ40, Aβ42, total Aβ. The second amyloid beta variant is identified on each row: Aβ38 (A, top row), Aβ40 (A, bottom row), Aβ42 (B, top row), total Aβ (B, bottom row). TTR ratio=tracer-to-tracee ratio, also described as ratio of labeled to unlabeled amyloid beta. Amyloid status: amyloid negative=blue, amyloid positive=red.

FIG. 12A-C depicts graphs showing the ratio of Aβ42 to other Aβ variants in amyloid negative (blue circles) and amyloid positive (red triangles) subjects. Specifically, (A) shows Aβ42:Aβ40 TTR ratio, (B) shows Aβ42:total Aβ TTR ratio, and (C) shows Aβ42:Aβ38 TTR ratio. Each datapoint represents an average of three measurements for each sample collected at either 1.5, 3, 9 and 24 hours after IV bolus and demonstrate increased Aβ42 relative to other isoforms during the increasing labeling phase (including, but not limited to, hours 1.5 & 3) and decreased Aβ42 relative to other isoforms during decreasing labeling phase (including, but not limited to, hours 9 & 24).

FIG. 13 depicts graphs showing normalized kinetic curves of plasma Aβ after administration of an IV bolus of ⁶C₁₃ leucine in amyloid negative (blue) and amyloid positive (red) subjects. (top graph) Aβ38, (second graph from the top), Aβ40 (third graph from the top) Aβ42, (bottom graph) total Aβ.

FIGS. 14A and B depicts graphs showing (A) Aβ42:Aβ40 TTR ratio (top graph), Aβ42:total Aβ TTR ratio (middle graph), and Aβ38:Aβ40 TTR ratio (bottom graph), (B) Aβ42:Aβ38 TTR ratio (top graph), Aβ38:total Aβ TTR ratio (middle graph), and Aβ40:total Aβ TTR ratio (bottom graph) after administration of an IV bolus of ⁶C₁₃ leucine in amyloid negative (blue line) and amyloid positive (red line) subjects. Data represent a single measurement of each sample, and mirror the general patterns seen in FIG. 12.

FIG. 15 depicts graphs showing normalized kinetic curves of plasma Aβ after administration of an oral bolus of ⁶C₁₃ leucine in amyloid negative (blue) subjects. (top graph) Aβ38, (second graph from the top), Aβ40 (third graph from the top) Aβ42, (bottom graph) total Aβ. Note: oral bolus labeling is similar to IV bolus labeling and may be used to detect amyloidosis.

FIG. 16A-D depicts graphs showing that Aβ42 kinetics are altered in brain amyloidosis. (A, B) SILK Aβ time course profiles of the isotopic enrichment of Aβ peptides normalized to plasma leucine for each participant of Aβ38, Aβ40, and Aβ42 show altered Aβ42 kinetics in the amyloid positive group only (mean±95% CI). (A) Amyloid negative participants (PET PIB MCP<0.18 or CSF Aβ42/Aβ40 concentration ratio>=0.12, n=51), (B) amyloid positive participants (n=49). Solid lines represent the mean model fit to the data. Blue: Aβ38; Green: Aβ40; Red: Aβ42. (C, D) SILK labeled Aβ isoform ratios #1 demonstrate altered Aβ42 kinetics in the amyloid positive group (D) (Blue: Aβ38/Aβ40 ratio; Red: Aβ42/Aβ40 ratio, mean±95% CI). The amyloid negative group (C) demonstrated similar kinetics of all three Aβ isoforms.

FIG. 17A-F depicts graphs showing that tertile analysis reveals completely normal SILK Aβ42 kinetics in participants with the highest CSF Aβ42/40 ratio and SILK Aβ42 alterations present at intermediate CSF Aβ42/40. (A, C, E) The mean and 95% confidence interval of the isotopic enrichment of Aβ peptides normalized to plasma leucine for each participant are shown. Solid lines represent the mean model fit to the data. Blue: Aβ38; Green: Aβ40; Red: A342. (B, D, F) The mean (±95% CI) of the ratio of Aβ isoforms labeled demonstrating differences in kinetics between Aβ isoforms when ratios≠1. Blue: Aβ38/Aβ40 ratio; Red: Aβ42/Aβ40 ratio. (A, B) n=34 participants with CSF Aβ42/40 concentration ratio>0.16; (C, D) n=34 participants with concentration ratio between 0.10-0.16; (E, F) right column is n=32 participants with concentration ratio<0.1. In the highest concentration ratio tertile group (A, B), the Aβ38, Aβ40 and Aβ42 time courses were virtually superimposable, with Aβ38/Aβ40 and Aβ42/Aβ40 enrichment ratios˜1.0 throughout the time course, which is considered “normal”. In contrast, there were clearly abnormal changes in Aβ42 kinetics in the lowest concentration ratio tertile group (E, F) and to a lesser extent in the middle tertile group (C, D). These results indicate that normal processing of soluble Aβ isoforms is handled identically between Aβ38, Aβ40, and Aβ42 as part of normal physiology. However, Aβ42 kinetics are altered in the presence of and possibly before significant amyloidosis is detectable by PET PIB or CSF Aβ42/Aβ40 ratios.

FIG. 18 is an illustration of a compartmental model of Aβ turnover kinetics.

FIG. 19A-F depicts graphs showing that Aβ38, Aβ40 and Aβ42 isoform turnover rates are slowed with increased age. (A-C) The time courses of labeled Aβ38 (A), Aβ40 (B) and Aβ42 (C) demonstrate slower turnover with increasing decade of life. Results are shown for the 100 subjects of the present study, plus 12 younger normal control subjects previously published. Results are averaged for amyloid negative participants only, separated into 4 age groups spanning decade ranges. Black open circle=age 30's-50's, n=10; red triangle=age 60's, n=25; green square=age 70's; blue closed circle=age 80's. Error bars represent 95% confidence intervals. Solid lines represent the average model fits to the data for each age group. (D-F) The turnover rates of Aβ38 (D), Aβ40 (E) and Aβ42 (F) are highly negatively correlated with increased age. Results from older amyloid negative (blue circles) and amyloid positive (red triangles) are shown with 12 younger amyloid negative participants (green circles). A linear fit with 95% CI are shown for the age vs. FTR of Aβ. There is a two-fold decrease in turnover rate from the 30's to the 70's.

FIG. 20A-F depicts graphs showing correlation figures of of Aβ SILK parameters (Aβ42/40 peak time ratios, (A) and (B); FTR Aβ42/40, (C) and (D); Aβ42 exchange rate, (E) and (F)) and measures of amyloidosis (PET PIB MCBP, (A), (C), (E); CSF Aβ42/40 concentration ratios, (B), (D), (F)). Correlation of Aβ SILK parameters and measures of amyloidosis identify linear correlations of Aβ42/40 peak time ratios and PET PIB MCBP, r=−0.47 and CSF Aβ42/40 concentration ratios, r=0.63. FTR Aβ42/40 and Aβ42 exchange demonstrate a non-linear or state change relationship to amyloidosis, suggesting these measures detect the presence of amyloidosis, but may not accurately quantify the amount.

FIG. 21A-C depicts graphs showing that fibrillar plaque growth is observed in cognitively normal participants, but plateaus with clinical dementia. (A) The annualized change in PIB MCBP is compared by baseline PIB and CDR. Delta PIB is the annualized change in PIB PET MCBP at times closest to the SILK study. All cognitively normal PIB+ participants (yellow squares) have increasing PIB signal (0.0493/year), while 4 of 6 cognitively impaired participants (red triangles) have no increase in PIB signal. These findings suggest that both fibrillar amyloid accumulation and Aβ42 FTR may have an interaction between clinical status and amyloid deposition. Reference red dotted line represents conditional cutoff for amyloid groups. (B) (C) Correlations between the annualized change in fibillar amyloid by PET PIB and FTR Aβ42 for all amyloid positive subjects (B) or all subjects (C) indicate that increasing fibrillar amyloid deposition is positively correlated with increased FTR Aβ42. In Amyloid positive (triangles) participants, R=0.75, p=0.002 and in both Amyloid positive and Amyloid negative (circles) participants, R=0.56, p=0.0002. The color shows CDR sum of the boxes (redder more impaired clinical dementia).

FIG. 22 is an illustration depicting a biological model for increased Aβ42 exchange and increased irreversible loss. Exchange and faster irreversible loss are present in amyloidosis (regardless of age, ApoE allele type or cognitive impairment), suggesting that amyloid plaques or associated higher-order Aβ structures (e.g. protofibrils or oligomers) underlie altered Aβ42 kinetics. The FTR may represent irreversible loss due to Aβ42 deposition on plaques, while Aβ42 exchange may represent interactions of newly generated soluble Aβ42 with higher order Aβ structures.

### DETAILED DESCRIPTION OF THE PREFERRED EMBODIMENTS

Applicants have unexpectedly discovered that the relative labeling of Aβ variants differs between healthy subjects and subjects with amyloid-6 (Aβ) amyloidosis at specific time points. The current invention provides methods for detecting, diagnosing, or monitoring the progression or treatment of Aβ amyloidosis. The term “Aβ amyloidosis’ refers to Aβ deposition in a subject that may result from differential metabolism (e.g. increased production, reduced clearance, or both). At the time of the invention, Aβ amyloidosis is clinically defined as evidence of Aβ deposition in the brain either by amyloid imaging (e.g. PiB PET) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Subjects with Aβ amyloidosis are also at an increased risk of developing a disease associated with Aβ amyloidosis. Diseases associated with Aβ amyloidosis include, but are not limited to, Alzheimer's Disease (AD), cerebral amyloid angiopathy, Lewy body dementia, and inclusion body myositis. Non-limiting examples of symptoms associated with Aβ amyloidosis may include impaired cognitive function, altered behavior, abnormal language function, emotional dysregulation, seizures, dementia, and impaired nervous system structure or function.

### I. Methods of the Invention

The present invention encompasses methods for detecting Aβ amyloidosis in a subject. Generally speaking, the method comprises measuring at one or more timepoints the in vivo relative labeling of at least two Aβ variants in a blood sample obtained from the subject, and calculating the ratio of relative labeling of a first Aβ variant to the relative labeling of a second Aβ variant, wherein a ratio other than 1 indicates the presence of Aβ amyloidosis. In at least some embodiments, Applicants have also contemplated using the amount of relative labeling of only a single Aβ variant at one or more timepoints to detect Aβ amyloidosis in a subject.

Methods of the invention may also be used for monitoring the progression or treatment of Aβ amyloidosis. The invention greatly enhances the accuracy of detection of Aβ amyloidosis early in the course of clinical disease or prior to the onset of brain damage and clinical symptoms in patients at risk of developing AD and monitoring the progression of the disease. Advantageously, as illustrated in the examples, the method allows for measuring Aβ dynamics in the blood without invasive spinal catheters. In addition, the method allows for specific testing of proposed disease modifying therapeutics which target Aβ.

### (a) Measuring the In Vivo Relative Labeling of Aβ Variants

In an aspect, the present invention provides means to measure the in vivo levels of one or more Aβ variants. For example, the in vivo levels of at least 1, at least 2, at least 3, at least 4, or at least 5 Aβ variants can be measured. In vivo levels of Aβ variants may be measured by labeling Aβ variants as they are synthesized in the central nervous system in vivo, and measuring in vitro the amount of one or more labeled and/or unlabeled Aβ variant over time (i.e. one or more time points) or at a single time in a blood sample obtained from a subject. These measurements may be used to calculate the in vivo relative labeling for an Aβ variant. As used herein, the phrase “relative labeling” refers to the ratio of labeled to unlabeled Aβ variant, the percent labeled Aβ variant, or both. Other kinetic parameters of Aβ metabolism may also be calculated for one or more Aβ variants based on the measurements of the in vivo levels of an Aβ variant. Non-limiting examples of other kinetic parameters of Aβ metabolism include the fractional synthesis rate, the fractional clearance rate, peak time, peak enrichment, initial downturn monoexponential slope, terminal monoexponential slope. Such methods are generally known in the art. For example, see U.S. Pat. No. 7,892,845, U.S. 20110294138, U.S. 20130115716, and Potter et al. Sci Transl Med 5(189), 2013, each hereby incorporated by reference in its entirety. Further details are also provided below.

In vivo levels of Aβ variants may be measured by measuring in vivo levels of labeled Aβ variants. In some embodiments, in vivo levels of labeled total Aβ protein may be measured. In other embodiments, in vivo levels of labeled Aβ40 may be measured. In yet other embodiments, in vivo levels of labeled Aβ42 may be measured. In still other embodiments, in vivo levels of labeled Aβ38 may be measured.

In vivo levels of Aβ variants may be measured by measuring in vivo levels of more than one labeled Aβ variant. In some embodiments, in vivo levels of labeled total Aβ protein and labeled Aβ42 may be measured. In other embodiments, in vivo levels of labeled total Aβ protein and labeled Aβ40 may be measured. In yet other embodiments, in vivo levels of labeled total Aβ protein and labeled Aβ38 may be measured. In additional embodiments, in vivo levels of labeled Aβ40 and labeled Aβ38 may be measured. In still other embodiments, in vivo levels of labeled Aβ42 and labeled Aβ38 may be measured. In different embodiments, in vivo levels of labeled Aβ42 protein and labeled Aβ40 may be measured. In other embodiments, in vivo levels of labeled total Aβ protein, labeled Aβ42 and labeled Aβ40 may be measured. In yet other embodiments, in vivo levels of labeled total Aβ protein, labeled Aβ40 and labeled Aβ38 may be measured. In still other embodiments, in vivo levels of labeled Aβ42, labeled Aβ40 and labeled Aβ38 may be measured. In additional embodiments, in vivo levels of labeled total Aβ protein, labeled Aβ42, labeled Aβ40 and labeled Aβ38 may be measured. In preferred embodiments, in vivo levels of labeled Aβ42 protein and labeled Aβ40 may be measured.

In vivo levels of Aβ variants may be measured by measuring in vivo levels of unlabeled Aβ variants. In some embodiments, in vivo levels of unlabeled total Aβ protein may be measured. In other embodiments, in vivo levels of unlabeled Aβ40 may be measured. In yet other embodiments, in vivo levels of unlabeled Aβ42 may be measured. In still other embodiments, in vivo levels of unlabeled Aβ38 may be measured.

In vivo levels of Aβ variants may be measured by measuring in vivo levels of more than one unlabeled Aβ variant. In some embodiments, in vivo levels of unlabeled total Aβ protein and unlabeled Aβ42 may be measured. In other embodiments, in vivo levels of unlabeled total Aβ protein and unlabeled Aβ40 may be measured. In yet other embodiments, in vivo levels of unlabeled total Aβ protein and unlabeled Aβ38 may be measured. In additional embodiments, in vivo levels of unlabeled Aβ40 and unlabeled Aβ38 may be measured. In still other embodiments, in vivo levels of unlabeled Aβ42 and unlabeled Aβ38 may be measured. In different embodiments, in vivo levels of unlabeled Aβ42 protein and unlabeled Aβ40 may be measured. In other embodiments, in vivo levels of unlabeled total Aβ protein, unlabeled Aβ42 and unlabeled Aβ40 may be measured. In yet other embodiments, in vivo levels of unlabeled total Aβ protein, unlabeled Aβ40 and unlabeled Aβ38 may be measured. In still other embodiments, in vivo levels of unlabeled Aβ42, unlabeled Aβ40 and unlabeled Aβ38 may be measured. In additional embodiments, in vivo levels of unlabeled total Aβ protein, unlabeled Aβ42, unlabeled Aβ40 and unlabeled Aβ38 may be measured. In preferred embodiments, in vivo levels of unlabeled Aβ42 protein and unlabeled Aβ40 may be measured.

In vivo levels of Aβ variants may be measured by measuring in vivo relative labeling of Aβ variants. As used herein, “in vivo relative labeling” refers to the percent of the variant that is labeled in vivo. Non-limiting examples of Aβ variants whose in vivo relative labeling may be measured may include total Aβ protein, Aβ40, Aβ42, or Aβ38. In some embodiments, in vivo relative labeling of total Aβ protein may be measured. In other embodiments, in vivo relative labeling of Aβ40 may be measured. In yet other embodiments, in vivo relative labeling of Aβ42 may be measured. In still other embodiments, in vivo relative labeling of Aβ38 may be measured.

In vivo relative labeling of more than one Aβ variant may be measured in vivo in the subject. In some embodiments, in vivo relative labeling of total Aβ protein and Aβ42 may be measured. In other embodiments, in vivo relative labeling of total Aβ protein and Aβ40 may be measured. In yet other embodiments, in vivo relative labeling of total Aβ protein and Aβ38 may be measured. In additional embodiments, in vivo relative labeling of Aβ40 protein and Aβ38 may be measured. In still other embodiments, in vivo relative labeling of Aβ42 and Aβ38 may be measured. In still other embodiments, in vivo relative labeling of Aβ42 and Aβ40 may be measured. In other embodiments, in vivo relative labeling of total Aβ protein, Aβ42 and Aβ40 may be measured. In yet other embodiments, in vivo relative labeling of total Aβ protein, Aβ40 and Aβ38 may be measured. In still other embodiments, in vivo relative labeling of total Aβ protein, Aβ42 and Aβ38 may be measured. In additional embodiments, in vivo relative labeling of Aβ42, Aβ40 and Aβ38 may be measured. In additional embodiments, in vivo relative labeling of total Aβ protein, Aβ42, Aβ40 and Aβ38 may be measured. In preferred embodiments, in vivo relative labeling of Aβ42 protein and Aβ40 may be measured.

Those of skill in the art will recognize that measuring one or more in vitro digestion products of an Aβ variant (e.g., Aβ₆₋₁₆, Aβ₁₇₋₂₈) may be used to determine the in vivo levels of an Aβ variant comprising the one or more Aβ in vitro digestion product. The specific in vitro digestion products of Aβ produced will depend on the endoprotease used. Non-limiting examples of suitable endoproteases include Glu-C, LysN, and trypsin. In some embodiments, in vivo levels of one or more Aβ variants may be measured by measuring one or more in vitro digestion product of an Aβ variant (e.g., Aβ₆₋₁₆, Aβ₁₇₋₂₈, Aβ₂₉₋₄₀, Aβ₂₉₋₄₂, Aβ₂₉₋₃₈ Aβ₂₈₋₄₂).

In vivo levels of Aβ variants may be measured by labeling Aβ variants as they are synthesized in the central nervous system in vivo, and measuring in vitro the amount of one or more labeled and/or labeled Aβ variant over time (i.e. one or more time points) or at a single time in a biological sample obtained from a subject. These measurements may be used to calculate the ratio of labeled to unlabeled Aβ variant, the percent labeled Aβ variant, or both. Other kinetic parameters of Aβ metabolism may also be calculated for one or more Aβ variants. Non-limiting examples of other kinetic parameters of Aβ metabolism include the fractional synthesis rate, the fractional clearance rate, peak time, peak enrichment, initial downturn monoexponential slope, terminal monoexponential slope. Such methods are generally known in the art. For example, see U.S. Pat. No. 7,892,845, U.S. 20110294138, and U.S. 20130115716, each hereby incorporated by reference in its entirety. Further details are also provided below.

i. Labeling Moiety

An Aβ variant may be labeled in vivo as it is synthesized in the central nervous system using a labeled moiety. Several different moieties may be used to label the Aβ variant. Generally speaking, the two types of labeling moieties typically utilized in the method of the invention are radioactive isotopes and non-radioactive (stable) isotopes. In a preferred embodiment, non-radioactive isotopes may be used and measured by mass spectrometry. Preferred stable isotopes include deuterium ²H, ¹³C, ¹⁵N, ^{17 or 18}O, ^{33, 34, or 36}S, but it is recognized that a number of other stable isotope that change the mass of an atom by more or less neutrons than is seen in the prevalent native form would also be effective. A suitable label generally will change the mass of the biomolecule under study such that it can be detected in a mass spectrometer. In one embodiment, the labeled moiety is an amino acid comprising a non-radioactive isotope (e.g., ¹³C). Alternatively, a radioactive isotope may be used, and the labeled biomolecules may be measured with a scintillation counter rather than a mass spectrometer. One or more labeled moieties may be used simultaneously or in sequence.

Those of skill in the art will appreciate that several amino acids may be used to provide the label of Aβ variant. Generally, the choice of amino acid is based on a variety of factors such as: (1) The amino acid generally is present in at least one residue of the Aβ variant. (2) The amino acid is generally able to quickly reach the site of protein synthesis and rapidly equilibrate across the blood-brain barrier. Leucine is a preferred amino acid to label proteins that are synthesized in the CNS such as Aβ variants. (3) The amino acid ideally may be an essential amino acid (not produced by the body), so that a higher percent of labeling may be achieved. Non-essential amino acids may also be used; however, measurements will likely be less accurate. (4) The amino acid label generally does not influence the metabolism of the protein of interest (e.g., very large doses of leucine may affect muscle metabolism). And (5) availability of the desired amino acid (i.e., some amino acids are much more expensive or harder to manufacture than others). In one embodiment, ¹³C₆-phenylalanine, which contains six ¹³C atoms, is used to label an Aβ variant. In a preferred embodiment, ¹³C₆-leucine is used to label an Aβ variant.

There are numerous commercial sources of labeled amino acids, both non-radioactive isotopes and radioactive isotopes. Generally, the labeled amino acids may be produced either biologically or synthetically. Biologically produced amino acids may be obtained from an organism (e.g., kelp/seaweed) grown in an enriched mixture of ¹³C, ¹⁵N, or another isotope that is incorporated into amino acids as the organism produces proteins. The amino acids are then separated and purified. Alternatively, amino acids may be made with known synthetic chemical processes.

ii. Administration of the Labeled Moiety

The method of the invention provides that the labeled moiety may be administered to a subject. Suitable subjects are described above. The labeled moiety may be administered to a subject by several methods. Suitable methods of administration include intravenously, intra-arterially, subcutaneously, intraperitoneally, intramuscularly, or orally. In a preferred embodiment, the labeled moiety is administered by intravenous infusion. In another preferred embodiment, the labeled moiety may be orally ingested.

The amount (or dose) of labeled moiety can and will vary, as can the duration and frequency of administration. A labeled moiety may be administered to a subject one or more times a day (e.g. 1, 2, 3, 4, 5 or more times a day) on one or more days (e.g. 1, 2, 3, 4, 5, 6, 7, 8, 9, 10 or more days). In each instance, the labeled moiety may be administered slowly over a period of time or as a single dose depending upon the type of analysis chosen (e.g., steady state or bolus). To achieve steady-state levels of the labeled Aβ variant, the labeling time generally should be of sufficient duration so that the labeled Aβ variant may be reliably quantified. The labeling time sufficient for reliable quantification of steady state levels of a labeled Aβ variant in a blood sample is typically less than required time for reliable quantification of steady state levels of the same Aβ variant in a CSF sample.

The amount of labeled Aβ variant is dependent upon (and estimated by) the percentage of label administered and the duration of labeling. Generally speaking, the amount of labeled Aβ variant will approximately equal the percentage of label administered multiplied by the duration of labeling. Stated another way, the amount of time of labeling is inversely related to the percent of the label amino acid compared to unlabeled amino acid (e.g. 10%, 50% or 100%). With less time labeling, more amount of labeled amino acid is required to achieve the same amount of Aβ variant labeling. Generally, the amount is dependent on (and estimated by) the following factors. (1) The type of analysis desired. For example, to achieve a steady state of about 15% labeled leucine in plasma requires about 2 mg/kg/hr over 9 hr after an initial bolus of about 3 mg/kg over 10 min. In contrast, if no steady state is required, a bolus of labeled leucine (e.g., about 400 mg to about 800 mg of labeled leucine) may be given. (2) The Aβ variant under analysis. For example, if the Aβ variant is being produced rapidly, then less labeling time may be needed and less label may be needed—perhaps as little as 100 mg or less as a bolus. And (3) the sensitivity of the technology to detect label. For example, as the sensitivity of label detection increases, the amount of label that is needed may decrease.

The amount of labeled Aβ variant needed for reliable quantification is a function of the sensitivity of the quantitation method. Current mass spectrometry methods can measure as low as approximately 0.01-0.2% labeled Aβ variant, though about 1% to about 2% labeled Aβ variant is preferred. However, these measurements are likely to improve (i.e. lower levels of labeled Aβ variant may be measured) with advances in technology. One skilled in the art will appreciate that the percent labeled Aβ variant needed for reliable quantification via other detection methods can readily be determined by routine experimentation, and labeling protocols can be modified based on the teachings herein.

In some embodiments, the labeled moiety is administered intravenously for an amount of time that is less than the half-life of Aβ in blood. In other embodiments, the labeled moiety is administered intravenously for an amount of time that is greater than the half-life of Aβ in blood. For example, the labeled moiety may be administered intravenously over a duration of minutes to hours, including, but not limited to, for at least 10 minutes, at least 20 minutes, at least 30 minutes, at least 1.0 hour, at least 1.5 hours, at least 2.0 hours, at least 2.5 hours, at least 3.0 hours, at least 3.5 hours, at least 4.0 hours, at least 4.5 hours, at least 5.0 hours, at least 5.5 hours, at least 6.0 hours, at least 6.5 hours, at least 7.0 hours, at least 7.5 hours, at least 8.0 hours, at least 8.5 hours, at least 9.0 hours, at least 9.5 hours, at least 10.0 hours, at least 10.5 hours, 1 at least 1.0 hours, at least 11.5 hours, or at least 12 hours. In other embodiments a labeled moiety is administered orally as multiple doses. The multiple doses may be administered sequentially or an amount of time may elapse between each dose. The amount of time between each dose may be a few seconds, a few minutes, or a few hours. In a preferred embodiment, when labeled amino acid is administered orally, it is provided to a subject as a drink. In each of the above embodiments, the labeled moiety can be labeled leucine, labeled phenylalanine, or any other labeled amino acid that is capable of crossing the blood brain barrier.

In some preferred embodiments, a labeled moiety is administered orally as a single bolus. In other preferred embodiments, a labeled moiety is administered intravenously as a single bolus. In still other preferred embodiments, a labeled moiety is administered intravenously as an infusion for about 1 hour. In still other preferred embodiments, a labeled moiety is administered intravenously as a bolus. As detailed in the Examples, all three methods of administration (oral bolus, IV bolus, and IV infusion) work equally well in terms of providing a reliable measure of amyloid beta metabolism. An intravenous bolus of a labeled moiety and an oral bolus of labeled moiety are easier to administer than an intravenous infusion, and also results in maximal levels of free label at an earlier time point (e.g. about 5 to about 10 minutes, and about 30 to about 60 minutes, respectively, for labeled leucine). In each of the above embodiments, the labeled moiety can be labeled leucine, labeled phenylalanine, or any other labeled amino acid that is capable of crossing the blood brain barrier.

Those of skill in the art will also appreciate that more than one label may be used in a single subject. This would allow multiple labeling of the same Aβ variant and may provide information on the production or clearance of that Aβ variant at different times. For example, a first label may be given to a subject over an initial time period, followed by a pharmacologic agent (drug), and then a second label may be administered. In general, analysis of the samples obtained from this subject would provide a measurement of metabolism before AND after drug administration, directly measuring the pharmacodynamic effect of the drug in the same subject.

Alternatively, multiple labels may be used at the same time to increase labeling of the Aβ variant, as well as obtain labeling of a broader range of Aβ variants.

iii. Biological Sample

The method of the invention provides that a biological sample be obtained from a subject so that the in vivo metabolism of the labeled Aβ variant may be determined. Suitable biological samples include, but are not limited to, cerebral spinal fluid (CSF), blood plasma, blood serum, urine, saliva, perspiration, and tears. In one embodiment of the invention, biological samples are taken from the CSF. In an alternate embodiment, biological samples are collected from the urine. In preferred embodiments, biological samples are collected from CSF or blood. As used herein, “blood” refers to either blood plasma or blood serum.

After administration of a labeled moiety, one or more samples will be collected from the subject. Cerebrospinal fluid may be obtained by lumbar puncture with or without an indwelling CSF catheter (a catheter is preferred if multiple collections are made over time). Blood may be collected by veni-puncture with or without an intravenous catheter, and processed according to methods known in the art. Urine may be collected by simple urine collection or more accurately with a catheter. Saliva and tears may be collected by direct collection using standard good manufacturing practice (GMP) methods. Samples may be used immediately or may be frozen and stored indefinitely.

As will be appreciated by those of skill in the art, the number of samples and when they would be taken generally will depend upon a number of factors such as: the type of analysis, type of administration, the Aβ variant of interest, the rate of metabolism, the type of detection, etc. In some embodiments, the invention provides that a first biological sample be taken from the subject prior to administration of the label to provide a baseline for the subject. In embodiments where a first biological sample is not taken from the subject prior to administration of the label, an assumption can be made that the baseline sample has a normal isotopic distribution.

The kinetic curve of Aβ labeling may be affected by the length of the labeling phase, although the kinetics of Aβ (e.g. production, clearance, turnover rates) will not substantially change. For example, labeled Aβ may peak earlier and lower following 6 hours of intravenous labeling compared to 9 hours of intravenous labeling. However, among a similar group of subjects (e.g. matched by age and/or disease status), the shape of the curve will generally be the same. Accordingly, one skilled in the art would be able to use the data provided herein to select a suitable sampling timeframe based on the labeling protocol.

In general, biological samples obtained during the labeling phase may be used to determine the rate of synthesis of the Aβ variant, and biological samples taken during the clearance phase may be used to determine the clearance rate of the Aβ variant. The amount of labeled Aβ detected in a biological sample increases during labeling and then decreases after the labeling has stopped. During the labeled Aβ increasing phase, some isoforms (e.g. Aβ42) increase more rapidly than other Aβ isoforms (for example Aβ40 or Aβ mid-domain). After labeling, during the labeled Aβ decreasing phase, some Aβ isoforms (e.g. Aβ42) decrease more rapidly than other Aβ isoforms (e.g. Aβ40 or Aβ mid-domain). Both the more rapid change of Aβ42 isoform during and after labeling indicate a more rapid metabolism, turn-over or half-life of Aβ42 compared to other isoforms. Therefore it is the unique metabolism rates of Aβ42 compared to other isoforms in blood that indicates amyloidosis. This is a surprising finding, in part, because blood Aβ isoform concentrations have not been predictive of amyloidosis in large controlled studies. The timeshift of Aβ42 isoform to other isoforms is largely independent of how the label is administered (oral vs. IV) or duration of labeling provided that kinetics of Aβ isoforms can be measured in blood.

In some embodiments, biological samples are taken hourly for 24 hours. Alternatively, biological samples may be taken every other hour or even less frequently. In other embodiments, biological samples are taken hourly for 16 hours. Alternatively, biological samples may be taken every other hour or even less frequently. In still other embodiments, a biological sample is taken during the production phase and a biological sample is taken during the clearance phase. In different embodiments, a biological sample is taken only during the production phase. In alternative embodiments, a biological sample is taken only during the clearance phase. In certain embodiments, at least 1, 2, 3, 4, 5, 6, 7, 8, 9, or 10 samples are taken during the production phase and/or at least 1, 2, 3, 4, 5, 6, 7, 8, 9, or 10 samples are taken during the clearance phase.

In some embodiments, one or more blood samples are taken about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes after administration of the labeled moiety. In some embodiments, one or more blood samples are taken about 0.5, about 1.0, about 1.5, about 2.0, about 2.5, about 3.0, about 3.5, or about 4 hours after administration of the labeled moiety. In other embodiments, one or more blood samples are taken about 4.0, about 4.5, about 5.0, about 5.5, about 6.0, about 6.5, about 7.0, about 7.5, about 8.0, about 8.5, about 9.0, about 9.5, about 10.0, about 10.5, about 11.0, about 11.5 or about 12.0 hours after administration of the labeled moiety. In other embodiments, one or more blood samples are taken about 10, about 11, about 12, about 13, about 14, about 15, about 16, about 17, about 18, about 19 or about 20 hours after administration of the labeled moiety. In other embodiments, one or more blood samples are taken about 16, about 17, about 18, about 19, about 20, about 21, about 22, about 23, or about 24 hours after administration of the labeled moiety. In other embodiments, one or more blood samples are taken about 1 minute to about 4 hours after administration of the labeled moiety. In other embodiments, one or more blood samples are taken about 10 minutes to about 4 hours after administration of the labeled moiety. In other embodiments, one or more blood samples are taken about 30 minutes to about 4 hours after administration of the labeled moiety. In other embodiments, one or more blood samples are taken about 1 minute to about 3 hours after administration of the labeled moiety. In other embodiments, one or more blood samples are taken about 10 minutes to about 3 hours after administration of the labeled moiety. In other embodiments, one or more blood samples are taken about 30 minutes to about 3 hours after administration of the labeled moiety. In other embodiments, one or more blood samples are taken about 1 hour to about 3 hours after administration of the labeled moiety. In other embodiments, one or more blood samples are taken about 4 to about 12 hours after administration of the labeled moiety. In other embodiments, one or more blood samples are taken about 4 to about 16 hours after administration of the labeled moiety. In other embodiments, one or more blood samples are taken about 9 to about 24 hours after administration of the labeled moiety. In other embodiments, one or more blood samples are taken about 16 to about 24 hours after administration of the labeled moiety. In other embodiments, one or more blood samples are taken about 1 minute to about 4 hours after administration of the labeled moiety and about 16 to about 24 hours after administration of the labeled moiety. In other embodiments, one or more blood samples are taken about 10 minutes to about 4 hours after administration of the labeled moiety and about 16 to about 24 hours after administration of the labeled moiety. In other embodiments, one or more blood samples are taken about 30 minutes to about 4 hours after administration of the labeled moiety and about 16 to about 24 hours after administration of the labeled moiety. In other embodiments, one or more blood samples are taken about 30 minutes to about 4 hours after administration of the labeled moiety and about 4 to about 12 hours after administration of the labeled moiety. In other embodiments, one or more blood samples are taken about 1 minute to about 4 hours after administration of the labeled moiety and about 4 to about 12 hours after administration of the labeled moiety. In other embodiments, one or more blood samples are taken about 10 minutes to about 4 hours after administration of the labeled moiety and about 4 to about 12 hours after administration of the labeled moiety. In other embodiments, one or more blood samples are taken about 30 minutes to about 4 hours after administration of the labeled moiety and about 4 to about 12 hours after administration of the labeled moiety.

iv. Detection

The present invention provides that detection of the amount of labeled Aβ and the amount of unlabeled Aβ in the biological sample may be detected. Suitable methods for the detection of labeled and unlabeled Aβ can and will vary according to the type of labeled moiety used to label it. If the labeled moiety is a non-radioactively labeled amino acid, then the method of detection typically should be sensitive enough to detect changes in mass of the labeled protein with respect to the unlabeled protein. In a preferred embodiment, mass spectrometry is used to detect differences in mass between the labeled and unlabeled Aβ. In one embodiment, gas chromatography mass spectrometry is used. In an alternate embodiment, MALDI-TOF mass spectrometry is used. In a preferred embodiment, high-resolution tandem mass spectrometry is used.

Additional techniques may be utilized to separate Aβ from other proteins and biomolecules in the biological sample. As an example, immunoprecipitation may be used to isolate and partially or completely purify Aβ before it is analyzed by mass spectrometry. Other methods of separating or concentrating Aβ may be used alone or in combination with immunoprecipitation. For example, chromatography techniques may be used to separate Aβ (or fragments thereof) by size, hydrophobicity or affinity. In particular, mass spectrometers having chromatography setups may be used to isolate proteins with or without immunoprecipitation, and then Aβ may be measured directly. In an exemplary embodiment, Aβ is immunoprecipitated and then analyzed by a liquid chromatography system interfaced with a tandem MS unit equipped with an electrospray ionization source (LC-ESI-tandem MS).

Labeled and unlabeled Aβ may also be cleaved into smaller peptides prior to detection. For instance, Aβ may be enzymatically cleaved with a protease to create several small peptides. Suitable proteases include, but are not limited to, trypsin, Lys-N, Lys-C, and Arg-N. In an exemplary embodiment, labeled and unlabeled Aβ is completely or partially purified from a biological sample, enzymatically cleaved with a protease, and then analyzed by a liquid chromatography system interfaced with a high-resolution tandem MS unit. In another exemplary embodiment, labeled and unlabeled Aβ is enzymatically cleaved with a protease and completely or partially purified, and then analyzed by a liquid chromatography system interfaced with a high-resolution tandem MS unit. In certain exemplary embodiments, Aβ is completely or partially purified by immunoprecipitation.

The invention also provides that multiple Aβ variants in the same biological sample may be measured simultaneously. That is, both the amount of unlabeled and labeled Aβ may be detected and measured separately or at the same time for multiple Aβ variants. As such, the invention provides a useful method for screening changes in synthesis and clearance of Aβ variants on a large scale (i.e. proteomics/metabolomics) and provides a sensitive means to detect and measure Aβ variants involved in the underlying pathophysiology of AD.

The amount of labeled tau in a biological sample at a given time reflects the metabolism of tau, including the synthesis rate (i.e., production) or the clearance rate (i.e., removal or destruction). Once the amount of labeled and unlabeled Aβ has been detected, the relative labeling of Aβ may be calculated. As used herein, “relative labeling” may refer to a ratio of labeled to unlabeled Aβ or the percent of labeled Aβ. The amount of labeled Aβ and/unlabeled Aβ may also be used to calculate one or more additional parameter of Aβ metabolism. Non-limiting examples of suitable metabolic parameters include the fractional synthesis rate, the fractional clearance rate, absolute synthesis rate, absolute clearance rate, fractional turnover rate, lag time, half-life, time to peak height, peak height, etc. Methods for calculating these parameters are well known in the art, and those of skill in the art will be familiar with the first order kinetic models of labeling that may be used with the method of the invention. In addition, other parameters, such as lag time and isotopic tracer steady state, may be determined and used as measurements of the protein's metabolism and physiology. Also, modeling may be performed on the data to fit multiple compartment models to estimate transfer between compartments. Of course, the type of mathematical modeling chosen will depend on the individual protein synthetic and clearance parameters (e.g., one-pool, multiple pools, steady state, non-steady-state, compartmental modeling, etc.). Parameters disclosed herein may be determined as described in WO 2014081851; WO 2006107814; Potter et al. Sci Transl Med 5(189), 2013; or Bateman et al. Nat Med 12, 2006, each of which is hereby incorporated by reference in its entirety. Generally, the relative labeling of Aβ in a biological sample is directly proportional to the metabolism of Aβ in the CNS. For example, the increase in labeled Aβ during the production phase and the removal of labeled Aβ during the clearance phase reflects the relative production and clearance of Aβ in the CNS. Accordingly, parameters of Aβ metabolism calculated using measurements of labeled and/or unlabeled Aβ also reflect the metabolism of Aβ in the CNS.

### (b) Calculating the Ratio of Relative Labeling and Detecting Aβ Amyloidosis

In another aspect, the present invention provides means for detecting Aβ amyloidosis by calculating the ratio of relative labeling of a first Aβ variant to the relative labeling of a second Aβ variant. Those of skill in the art will recognize that, when the relative labeling of any two Aβ variants are similar in a given sample, the ratio of relative labeling of said two Aβ variants may be about 1. Conversely, when the relative labeling of any one Aβ variant differs from the relative labeling of other Aβ variants in a given sample, the ratio of relative labeling of said Aβ variant to another Aβ variant in the biological sample may be a number other than 1 (i.e. greater than about 1 or less than about 1). As illustrated in the examples, the inventors discovered that the relative labeling of Aβ variants in healthy subjects are similar in a given sample taken at a given time. Therefore, the ratio of relative labeling of any Aβ variant to any other Aβ variant in a healthy subject may be about 1. Surprisingly, the inventors also discovered that the relative labeling of a first Aβ variant in a subject with Aβ amyloidosis is different from the relative labeling of other Aβ variants in a given sample taken at a given time. Therefore, the ratio of relative labeling of a first Aβ variant to any other Aβ variant in a subject with Aβ amyloidosis may be a number other than about 1. In other words, a ratio of relative labeling of a first Aβ variant to any other Aβ variant in a subject of about 1 indicates the absence of Aβ amyloidosis. Conversely, a ratio of relative labeling of a first Aβ variant to any other Aβ variant in a subject is other than one 1, indicates the presence of Aβ amyloidosis.

In some embodiments, the ratio of relative labeling of total Aβ to Aβ42 may be measured. In other embodiments, the ratio of relative labeling of Aβ42 to total Aβ may be measured. In yet other embodiments, the ratio of relative labeling of total Aβ to Aβ40 may be measured. In additional embodiments, the ratio of relative labeling of Aβ40 to total Aβ may be measured. In still other embodiments, the ratio of relative labeling of total Aβ to Aβ38 may be measured. In other embodiments, the ratio of relative labeling of Aβ38 to total Aβ may be measured. In additional embodiments, the ratio of relative labeling of Aβ40 to Aβ38 may be measured. In yet embodiments, the ratio of relative labeling of Aβ38 to Aβ40 may be measured. In still other embodiments, the ratio of relative labeling of Aβ42 to Aβ38 may be measured. In other embodiments, the ratio of relative labeling of Aβ38 to Aβ42 may be measured. In some embodiments, the ratio of relative labeling of Aβ40 to Aβ42 may be measured. In preferred embodiments, the ratio of relative labeling of Aβ42 to Aβ40 may be measured.

As described in the examples, Aβ42 labeling preceded the labeling of the other Aβ variants during the production phase and fell faster than the other Aβ variants during the clearance phase in subjects with Aβ amyloidosis. Thus, a significantly higher rate of metabolism and turnover for Aβ42 can be measured compared to other Aβ variants in blood from a subject with Aβ amyloidosis. This is also reflected in the ratio of relative labeling of Aβ42 to other Aβ variants. Stated another way, the relative labeling of Aβ42 in subjects with Aβ amyloidosis may be higher than the relative labeling of other Aβ variants in blood samples taken during the production phase, and less than the relative labeling of other Aβ variants in blood samples taken during the clearance phase. Thus, the relative labeling of Aβ42 in subjects with Aβ amyloidosis may be higher than the relative labeling of other Aβ variants in blood samples taken at about 1 minute to about 4 hours after the start of bolus labeling, and less than the relative labeling of other Aβ variants in blood samples taken at about 4 to about 24 hours after the start of bolus labeling. Therefore, the ratio of relative labeling of Aβ42 to other Aβ variants in blood samples taken at about 1 minute to about 4 hours may be greater than about 1, and the ratio of relative labeling of Aβ42 to other Aβ variants in blood samples taken at about 4 to about 24 hours may be less than about 1. If a longer labeling protocol is used (e.g. IV infusion), the relative labeling of Aβ42 in subjects with Aβ amyloidosis may be higher than the relative labeling of other Aβ variants in blood samples taken after 4 hours and may be less than the relative labeling of other Aβ variants in blood samples taken after 24 hours. A skilled artisan will appreciate that while the Applicants have generalized their statements as a ratio of Aβ42 to another Aβ variant, the reverse is also true. Thus, the ratio of relative labeling of other Aβ variants to Aβ42 in blood samples taken at about 1 minute to about 4 hours may be less than about 1, and the ratio of relative labeling of other Aβ variants to Aβ42 in blood samples taken at about 4 to about 24 hours may be greater than about 1. Further, any comparison of the amount of a first labeled Aβ isoform to a second labeled Aβ isoform can indicate amyloidosis. Non-limiting means by which a comparison can be performed include by subtraction, by an algorithm or other any other calculation known in the art.

### (i) Relative Labeling of Aβ42 to Aβ40

In some embodiments, a ratio of relative labeling of Aβ42 to Aβ40 greater than about 1 in a blood sample taken at about 1 minute to about 4 hours indicates the presence of Aβ amyloidosis. In other embodiments, a ratio of relative labeling of Aβ42 to Aβ40 greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ42 to Aβ40 greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ42 to Aβ40 greater than about 1 in a blood sample taken at about 10 minutes to about 4 hours indicates the presence of Aβ amyloidosis. In different embodiments, a ratio of relative labeling of Aβ42 to Aβ40 greater than about 1 in a blood sample taken at about 30 minutes to about 4 hours indicates the presence of Aβ amyloidosis. In different embodiments, a ratio of relative labeling of Aβ42 to Aβ40 greater than about 1 in a blood sample taken at about 1 to about 4 hours indicates the presence of Aβ amyloidosis. In still different embodiments, a ratio of relative labeling of Aβ42 to Aβ40 greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ42 to Aβ40 greater than about 1 in a blood sample taken at about 1 minutes to about 3 hours indicates the presence of Aβ amyloidosis. In still additional embodiments, a ratio of relative labeling of Aβ42 to Aβ40 greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ42 to Aβ40 greater than about 1 in a blood sample taken at about 1.5 to about 3 hours indicates the presence of Aβ amyloidosis. In alternative embodiments, a ratio of relative labeling of Aβ42 to Aβ40 greater than about 1 in a blood sample taken at about 1.5, about 2.0, about 2.5, or about 3.0 indicates the presence of Aβ amyloidosis. In a preferred embodiment, a ratio of relative labeling of Aβ42 to Aβ40 greater than about 1 in a blood sample taken at about 3 hours, indicates the presence of Aβ amyloidosis.

In some embodiments, a ratio of relative labeling of Aβ42 to Aβ40 may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of Aβ42 to A640 may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of Aβ42 to A640 may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of Aβ42 to A640 may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of Aβ42 to A640 may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of Aβ42 to A640 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 1 minute indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 1 minute indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 10 minutes indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 10 minutes indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 0.5 hour indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 0.5 hour indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 1 hour indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 1 hour indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 1.5 hours indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 1.5 hours indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 2 hours indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 2 hours indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 4 hours indicates the presence of Aβ amyloidosis. In yet other embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 4 hours indicates the presence of Aβ amyloidosis. In additional embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 4 hours indicates the presence of Aβ amyloidosis. In other embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 4 hours indicates the presence of Aβ amyloidosis.

In some preferred embodiments, a ratio of relative labeling of Aβ42 to Aβ40 of about 1.10, about 1.15, about 1.16, about 1.17, about 1.18, about 1.19, about 1.20, about 1.21, about 1.22, about 1.23, about 1.24, about 1.25 or above in a blood sample taken at about 3 hours indicates the presence of Aβ amyloidosis. In other preferred embodiments, a ratio of relative labeling of Aβ42 to Aβ40 may about 1.20, about 1.21, about 1.22, about 1.23, about 1.24, about 1.25, about 1.26, about 1.27, about 1.28, about 1.29, about 1.30 in a blood sample taken at about 3 hours indicates the presence of Aβ amyloidosis. In still other preferred embodiments, a ratio of relative labeling of Aβ42 to Aβ40 may be about 1.2, about 1.3, about 1.4, about 1.5, about 1.6, about 1.7, about 1.8, about 1.9, or about 2.0 in a blood sample taken at about 3 hours indicates the presence of Aβ amyloidosis. In additional preferred embodiments, a ratio of relative labeling of Aβ42 to Aβ40 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

As described in the examples, the relative labeling of the Aβ42 variant in subjects with Aβ amyloidosis may be lower than the relative labeling of other Aβ variants in blood samples taken at about 4 to about 24 hours. Therefore, the ratio of relative labeling of Aβ42 to Aβ40 in blood samples taken at about 4 to about 24 hours may be lower than about 1.

In some embodiments, a ratio of relative labeling of Aβ42 to Aβ40 less than about 1 in a blood sample taken at about 4 to about 24 hours indicates the presence of Aβ amyloidosis. In other embodiments, a ratio of relative labeling of Aβ42 to Aβ40 less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ42 to Aβ40 less than about 1 in a blood sample taken at about 9 to about 24 hours indicates the presence of Aβ amyloidosis. In other embodiments, a ratio of relative labeling of Aβ42 to Aβ40 less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ42 to Aβ40 less than about 1 in a blood sample taken at about 12 to about 24 hours indicates the presence of Aβ amyloidosis. In further embodiments, a ratio of relative labeling of Aβ42 to Aβ40 less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ42 to Aβ40 less than about 1 in a blood sample taken at about 16 to about 24 hours indicates the presence of Aβ amyloidosis. In still other embodiments, a ratio of relative labeling of Aβ42 to Aβ40 less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ42 to Aβ40 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ42 to Aβ40 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ42 to Aβ40 may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ42 to Aβ40 less than about 1 in a blood sample taken at about 6 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ42 to Aβ40 less than about 1 in a blood sample taken at about 9 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ42 to Aβ40 less than about 1 in a blood sample taken at about 12 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ42 to Aβ40 less than about 1 in a blood sample taken at about 16 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ42 to Aβ40 less than about 1 in a blood sample taken at about 20 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ42 to Aβ40 less than about 1 in a blood sample taken at about 24 hours indicates the presence of Aβ amyloidosis. In one embodiment, a ratio of relative labeling of Aβ42 to Aβ40 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ42 to Aβ40 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ42 to Aβ40 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ42 to Aβ40 less than about 1 in a blood sample taken at about 9 hours indicates the presence of Aβ amyloidosis. In another preferred embodiment, a ratio of relative labeling of Aβ42 to Aβ40 may be about 0.85, 0.80, 0.75, 0.70, 0.65, 0.60, 0.55, or about 0.50 in a blood sample taken at about 9 hours, indicating the presence of Aβ amyloidosis. In yet another preferred embodiment, a ratio of relative labeling of Aβ42 to Aβ40 less than about 1 in a blood sample taken at about 24 hours indicates the presence of Aβ amyloidosis. In still another preferred embodiment, a ratio of relative labeling of Aβ42 to Aβ40 may be about 0.85, 0.80, 0.75, 0.70, 0.65, 0.60, 0.55, or about 0.50 in a blood sample taken at about 24 hours, indicating the presence of Aβ amyloidosis.

### (ii) Relative Labeling of Aβ42 to Aβ38

In some embodiments, a ratio of relative labeling of Aβ42 to Aβ38 greater than about 1 in a blood sample taken at about 1 minute to about 4 hours indicates the presence of Aβ amyloidosis. In other embodiments, a ratio of relative labeling of Aβ42 to Aβ38 greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ42 to Aβ38 greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ42 to Aβ38 greater than about 1 in a blood sample taken at about 10 minutes to about 4 hours indicates the presence of Aβ amyloidosis. In different embodiments, a ratio of relative labeling of Aβ42 to Aβ38 greater than about 1 in a blood sample taken at about 30 minutes to about 4 hours indicates the presence of Aβ amyloidosis. In different embodiments, a ratio of relative labeling of Aβ42 to Aβ38 greater than about 1 in a blood sample taken at about 1 to about 4 hours indicates the presence of Aβ amyloidosis. In still different embodiments, a ratio of relative labeling of Aβ42 to Aβ38 greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ42 to Aβ38 greater than about 1 in a blood sample taken at about 1 minutes to about 3 hours indicates the presence of Aβ amyloidosis. In still additional embodiments, a ratio of relative labeling of Aβ42 to Aβ38 greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ42 to Aβ38 greater than about 1 in a blood sample taken at about 1.5 to about 3 hours indicates the presence of Aβ amyloidosis. In alternative embodiments, a ratio of relative labeling of Aβ42 to Aβ38 greater than about 1 in a blood sample taken at about 1.5, about 2.0, about 2.5, or about 3.0 indicates the presence of Aβ amyloidosis. In a preferred embodiment, a ratio of relative labeling of Aβ42 to Aβ38 greater than about 1 in a blood sample taken at about 3 hours, indicates the presence of Aβ amyloidosis.

In some embodiments, a ratio of relative labeling of Aβ42 to Aβ38 may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of Aβ42 to A638 may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of Aβ42 to A638 may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of Aβ42 to A638 may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of Aβ42 to A638 may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In additional embodiments, a ratio of relative labeling of Aβ42 to A638 may be about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 1 minute indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 1 minute indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 1 minute indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 1 minute indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 10 minutes indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 10 minutes indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 10 minutes indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 10 minutes indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 0.5 hour indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 0.5 hour indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 0.5 hour indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 0.5 hour indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 1.0 hour indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 1 hour indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 1 hour indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 1 hour indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 1.5 hour indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 1.5 hours indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 1.5 hours indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 1.5 hours indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 2 hours indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 2 hours indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 2 hours indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 2 hours indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 2.5 hours indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 2.5 hours indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 2.5 hours indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 2.5 hours indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 2.5 hours indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2.5 hours indicates the presence of Aβ amyloidosis.

In some embodiments, a ratio of relative labeling of Aβ42 to Aβ38 of about 1.10, about 1.20, about 1.21, about 1.22, about 1.23, about 1.24, about 1.25, about 1.26, about 1.27, about 1.28, about 1.29, about 1.30 or above in a blood sample taken at about 3 hours indicates the presence of Aβ amyloidosis. In other embodiments, a ratio of relative labeling of Aβ42 to Aβ38 of about 1.30, about 1.31, about 1.32, about 1.33, about 1.34, about 1.35, about 1.36, about 1.37, about 1.38, about 1.39, about 1.40 or above in a blood sample taken at about 3 hours indicates the presence of Aβ amyloidosis. In yet other embodiments, a ratio of relative labeling of Aβ42 to Aβ38 may about 1.40, about 1.41, about 1.42, about 1.43, about 1.44, about 1.45, about 1.46, about 1.47, about 1.48, about 1.49, about 1.50 in a blood sample taken at about 3 hours indicates the presence of Aβ amyloidosis. In additional embodiments, a ratio of relative labeling of Aβ42 to Aβ38 may be about 1.3, about 1.4, about 1.5, about 1.6, about 1.7, about 1.8, about 1.9, or about 2.0 in a blood sample taken at about 3 hours indicates the presence of Aβ amyloidosis. In additional embodiments, a ratio of relative labeling of Aβ42 to Aβ38 may be about 1.5, about 1.6, about 1.7, about 1.8, about 1.9, or about 2.0 in a blood sample taken at about 3 hours indicates the presence of Aβ amyloidosis. In other embodiments, a ratio of relative labeling of Aβ42 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

As described in the examples, the relative labeling of the Aβ42 variant in subjects with Aβ amyloidosis may be lower than the relative labeling of other Aβ variants in blood samples taken at about 4 to about 24 hours. Therefore, the ratio of relative labeling of Aβ42 to Aβ38 in blood samples taken at about 4 to about 24 hours may be lower than about 1.

In some embodiments, a ratio of relative labeling of Aβ42 to Aβ38 less than about 1 in a blood sample taken at about 4 to about 24 hours indicates the presence of Aβ amyloidosis. In other embodiments, a ratio of relative labeling of Aβ42 to Aβ38 less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ42 to Aβ38 less than about 1 in a blood sample taken at about 9 to about 24 hours indicates the presence of Aβ amyloidosis. In other embodiments, a ratio of relative labeling of Aβ42 to Aβ38 less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ42 to Aβ38 less than about 1 in a blood sample taken at about 12 to about 24 hours indicates the presence of Aβ amyloidosis. In further embodiments, a ratio of relative labeling of Aβ42 to Aβ38 less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ42 to Aβ38 less than about 1 in a blood sample taken at about 16 to about 24 hours indicates the presence of Aβ amyloidosis. In still other embodiments, a ratio of relative labeling of Aβ42 to Aβ38 less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ42 to Aβ38 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ42 to Aβ38 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ42 to Aβ38 may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ42 to Aβ38 less than about 1 in a blood sample taken at about 6 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ42 to Aβ38 less than about 1 in a blood sample taken at about 9 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ42 to Aβ38 less than about 1 in a blood sample taken at about 12 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ42 to Aβ38 less than about 1 in a blood sample taken at about 16 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ42 to Aβ38 less than about 1 in a blood sample taken at about 20 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ42 to Aβ38 less than about 1 in a blood sample taken at about 24 hours indicates the presence of Aβ amyloidosis. In one embodiment, a ratio of relative labeling of Aβ42 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ42 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ42 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ42 to Aβ38 less than about 1 in a blood sample taken at about 9 hours indicates the presence of Aβ amyloidosis. In another preferred embodiment, a ratio of relative labeling of Aβ42 to Aβ38 may be about 0.85, 0.80, 0.75, 0.70, 0.65, 0.60, 0.55, or about 0.50 in a blood sample taken at about 9 hours, indicating the presence of Aβ amyloidosis. In yet another preferred embodiment, a ratio of relative labeling of Aβ42 to Aβ38 less than about 1 in a blood sample taken at about 24 hours indicates the presence of Aβ amyloidosis. In still another preferred embodiment, a ratio of relative labeling of Aβ42 to Aβ38 may be about 0.85, 0.80, 0.75, 0.70, 0.65, 0.60, 0.55, or about 0.50 in a blood sample taken at about 24 hours, indicating the presence of Aβ amyloidosis.

### (iii) Relative Labeling of Aβ42 to Total Aβ

In some embodiments, a ratio of relative labeling of Aβ42 to total Aβ greater than about 1 in a blood sample taken at about 1 minute to about 4 hours indicates the presence of Aβ amyloidosis. In other embodiments, a ratio of relative labeling of Aβ42 to total Aβ greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ42 to total Aβ greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ42 to total Aβ greater than about 1 in a blood sample taken at about 10 minutes to about 4 hours indicates the presence of Aβ amyloidosis. In different embodiments, a ratio of relative labeling of Aβ42 to total Aβ greater than about 1 in a blood sample taken at about 30 minutes to about 4 hours indicates the presence of Aβ amyloidosis. In different embodiments, a ratio of relative labeling of Aβ42 to total Aβ greater than about 1 in a blood sample taken at about 1 to about 4 hours indicates the presence of Aβ amyloidosis. In still different embodiments, a ratio of relative labeling of Aβ42 to total Aβ greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ42 to Total Aβ greater than about 1 in a blood sample taken at about 1 minutes to about 3 hours indicates the presence of Aβ amyloidosis. In still additional embodiments, a ratio of relative labeling of Aβ42 to total Aβ greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ42 to total Aβ greater than about 1 in a blood sample taken at about 1.5 to about 3 hours indicates the presence of Aβ amyloidosis. In alternative embodiments, a ratio of relative labeling of Aβ42 to total Aβ greater than about 1 in a blood sample taken at about 1.5, about 2.0, about 2.5, or about 3.0 indicates the presence of Aβ amyloidosis. In a preferred embodiment, a ratio of relative labeling of Aβ42 to total Aβ greater than about 1 in a blood sample taken at about 3 hours, indicates the presence of Aβ amyloidosis.

In some embodiments, a ratio of relative labeling of Aβ42 to total Aβ may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of Aβ42 to total Aβ may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of Aβ42 to total Aβ may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 minute indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 1 minute indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 10 minutes indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 10 minutes indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 0.5 hour indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 0.5 hour indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 hour indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 1 hour indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1.5 hours indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 1.5 hours indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 2 hours indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 2 hours indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 4 hours indicates the presence of Aβ amyloidosis. In yet other embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 4 hours indicates the presence of Aβ amyloidosis. In additional embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 4 hours indicates the presence of Aβ amyloidosis. In other embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 4 hours indicates the presence of Aβ amyloidosis.

In some preferred embodiments, a ratio of relative labeling of Aβ42 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 3 hours indicates the presence of Aβ amyloidosis. In other preferred embodiments, a ratio of relative labeling of Aβ42 to total Aβ may about 1.20, about 1.21, about 1.22, about 1.23, about 1.24, about 1.25, about 1.26, about 1.27, about 1.28, about 1.29, about 1.30 in a blood sample taken at about 3 hours indicates the presence of Aβ amyloidosis. In still other preferred embodiments, a ratio of relative labeling of Aβ42 to total Aβ may be about 1.2, about 1.3, about 1.4, about 1.5, about 1.6, about 1.7, about 1.8, about 1.9, or about 2.0 in a blood sample taken at about 3 hours indicates the presence of Aβ amyloidosis. In additional preferred embodiments, a ratio of relative labeling of Aβ42 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

As described in the examples, the relative labeling of the Aβ42 variant in subjects with Aβ amyloidosis may be lower than the relative labeling of other Aβ variants in blood samples taken at about 4 to about 24 hours. Therefore, the ratio of relative labeling of Aβ42 to total Aβ in blood samples taken at about 4 to about 24 hours may be lower than about 1.

In some embodiments, a ratio of relative labeling of Aβ42 to total Aβ less than about 1 in a blood sample taken at about 4 to about 24 hours indicates the presence of Aβ amyloidosis. In other embodiments, a ratio of relative labeling of Aβ42 to total Aβ less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ42 to total Aβ less than about 1 in a blood sample taken at about 9 to about 24 hours indicates the presence of Aβ amyloidosis. In other embodiments, a ratio of relative labeling of Aβ42 to total Aβ less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ42 to total Aβ less than about 1 in a blood sample taken at about 12 to about 24 hours indicates the presence of Aβ amyloidosis. In further embodiments, a ratio of relative labeling of Aβ42 to total Aβ less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ42 to total Aβ less than about 1 in a blood sample taken at about 16 to about 24 hours indicates the presence of Aβ amyloidosis. In still other embodiments, a ratio of relative labeling of Aβ42 to total Aβ less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ42 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ42 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ42 to total Aβ may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ42 to total Aβ less than about 1 in a blood sample taken at about 6 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ42 to total Aβ less than about 1 in a blood sample taken at about 9 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ42 to total Aβ less than about 1 in a blood sample taken at about 12 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ42 to total Aβ less than about 1 in a blood sample taken at about 16 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ42 to total Aβ less than about 1 in a blood sample taken at about 20 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ42 to total Aβ less than about 1 in a blood sample taken at about 24 hours indicates the presence of Aβ amyloidosis. In one embodiment, a ratio of relative labeling of Aβ42 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ42 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ42 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ42 to total Aβ less than about 1 in a blood sample taken at about 9 hours indicates the presence of Aβ amyloidosis. In another preferred embodiment, a ratio of relative labeling of Aβ42 to total Aβ may be about 0.85, 0.80, 0.75, 0.70, 0.65, 0.60, 0.55, or about 0.50 in a blood sample taken at about 9 hours, indicating the presence of Aβ amyloidosis. In a preferred embodiment, a ratio of relative labeling of Aβ42 to total Aβ less than about 1 in a blood sample taken at about 24 hours indicates the presence of Aβ amyloidosis. In yet another preferred embodiment, a ratio of relative labeling of Aβ42 to total Aβ may be about 0.85, 0.80, 0.75, 0.70, 0.65, 0.60, 0.55, or about 0.50 in a blood sample taken at about 24 hours, indicating the presence of Aβ amyloidosis.

### (iv) Relative Labeling of Aβ40 to Aβ38

In some embodiments, a ratio of relative labeling of Aβ40 to Aβ38 greater than about 1 in a blood sample taken at about 1 minute to about 4 hours indicates the presence of Aβ amyloidosis. In other embodiments, a ratio of relative labeling of Aβ40 to Aβ38 greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ40 to Aβ38 greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ40 to Aβ38 greater than about 1 in a blood sample taken at about 10 minutes to about 4 hours indicates the presence of Aβ amyloidosis. In different embodiments, a ratio of relative labeling of Aβ40 to Aβ38 greater than about 1 in a blood sample taken at about 30 minutes to about 4 hours indicates the presence of Aβ amyloidosis. In different embodiments, a ratio of relative labeling of Aβ40 to Aβ38 greater than about 1 in a blood sample taken at about 1 to about 4 hours indicates the presence of Aβ amyloidosis. In still different embodiments, a ratio of relative labeling of Aβ40 to Aβ38 greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ40 to Aβ38 greater than about 1 in a blood sample taken at about 1 minutes to about 3 hours indicates the presence of Aβ amyloidosis. In still additional embodiments, a ratio of relative labeling of Aβ40 to Aβ38 greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ40 to Aβ38 greater than about 1 in a blood sample taken at about 1.5 to about 3 hours indicates the presence of Aβ amyloidosis. In alternative embodiments, a ratio of relative labeling of Aβ40 to Aβ38 greater than about 1 in a blood sample taken at about 1.5, about 2.0, about 2.5, or about 3.0 indicates the presence of Aβ amyloidosis. In a preferred embodiment, a ratio of relative labeling of Aβ40 to Aβ38 greater than about 1 in a blood sample taken at about 3 hours, indicates the presence of Aβ amyloidosis.

In some embodiments, a ratio of relative labeling of Aβ40 to Aβ38 may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of A640 to A638 may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of A640 to A638 may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of A640 to A638 may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of A640 to A638 may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of A640 to A638 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 1 minute indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 1 minute indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 10 minutes indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 10 minutes indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 0.5 hour indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 0.5 hour indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 1 hour indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 1 hour indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 1.5 hours indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 1.5 hours indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 2 hours indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 2 hours indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 4 hours indicates the presence of Aβ amyloidosis. In yet other embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 4 hours indicates the presence of Aβ amyloidosis. In additional embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 4 hours indicates the presence of Aβ amyloidosis. In other embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 4 hours indicates the presence of Aβ amyloidosis.

In some preferred embodiments, a ratio of relative labeling of Aβ40 to Aβ38 of about 1.10, about 1.15, about 1.16, about 1.17, about 1.18, about 1.19, about 1.20, about 1.21, about 1.22, about 1.23, about 1.24, about 1.25 or above in a blood sample taken at about 3 hours indicates the presence of Aβ amyloidosis. In other preferred embodiments, a ratio of relative labeling of Aβ40 to Aβ38 may about 1.20, about 1.21, about 1.22, about 1.23, about 1.24, about 1.25, about 1.26, about 1.27, about 1.28, about 1.29, about 1.30 in a blood sample taken at about 3 hours indicates the presence of Aβ amyloidosis. In still other preferred embodiments, a ratio of relative labeling of Aβ40 to Aβ38 may be about 1.2, about 1.3, about 1.4, about 1.5, about 1.6, about 1.7, about 1.8, about 1.9, or about 2.0 in a blood sample taken at about 3 hours indicates the presence of Aβ amyloidosis. In additional preferred embodiments, a ratio of relative labeling of Aβ40 to Aβ38 may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

In some embodiments, a ratio of relative labeling of Aβ40 to Aβ38 less than about 1 in a blood sample taken at about 4 to about 24 hours indicates the presence of Aβ amyloidosis. In other embodiments, a ratio of relative labeling of Aβ40 to Aβ38 less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ40 to Aβ38 less than about 1 in a blood sample taken at about 9 to about 24 hours indicates the presence of Aβ amyloidosis. In other embodiments, a ratio of relative labeling of Aβ40 to Aβ38 less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ40 to Aβ38 less than about 1 in a blood sample taken at about 12 to about 24 hours indicates the presence of Aβ amyloidosis. In further embodiments, a ratio of relative labeling of Aβ40 to Aβ38 less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ40 to Aβ38 less than about 1 in a blood sample taken at about 16 to about 24 hours indicates the presence of Aβ amyloidosis. In still other embodiments, a ratio of relative labeling of Aβ40 to Aβ38 less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ40 to Aβ38 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ40 to Aβ38 may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ40 to Aβ38 may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ40 to Aβ38 less than about 1 in a blood sample taken at about 6 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ40 to Aβ38 less than about 1 in a blood sample taken at about 9 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ40 to Aβ38 less than about 1 in a blood sample taken at about 12 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ40 to Aβ38 less than about 1 in a blood sample taken at about 16 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ40 to Aβ38 less than about 1 in a blood sample taken at about 20 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ40 to Aβ38 less than about 1 in a blood sample taken at about 24 hours indicates the presence of Aβ amyloidosis. In one embodiment, a ratio of relative labeling of Aβ40 to Aβ38 may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ40 to Aβ38 may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ40 to Aβ38 may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ40 to Aβ38 less than about 1 in a blood sample taken at about 9 hours indicates the presence of Aβ amyloidosis. In yet another preferred embodiment, a ratio of relative labeling of Aβ40 to Aβ38 may be about 0.85, 0.80, 0.75, 0.70, 0.65, 0.60, 0.55, or about 0.50 in a blood sample taken at about 9 hours, indicating the presence of Aβ amyloidosis. In a preferred embodiment, a ratio of relative labeling of Aβ40 to Aβ38 less than about 1 in a blood sample taken at about 24 hours indicates the presence of Aβ amyloidosis. In yet another preferred embodiment, a ratio of relative labeling of Aβ40 to Aβ38 may be about 0.85, 0.80, 0.75, 0.70, 0.65, 0.60, 0.55, or about 0.50 in a blood sample taken at about 24 hours, indicating the presence of Aβ amyloidosis.

### (v) Relative Labeling of Aβ40 to Total Aβ

In some embodiments, a ratio of relative labeling of Aβ40 to total Aβ greater than about 1 in a blood sample taken at about 1 minute to about 4 hours indicates the presence of Aβ amyloidosis. In other embodiments, a ratio of relative labeling of Aβ40 to total Aβ greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ40 to total Aβ greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ40 to total Aβ greater than about 1 in a blood sample taken at about 10 minutes to about 4 hours indicates the presence of Aβ amyloidosis. In different embodiments, a ratio of relative labeling of Aβ40 to total Aβ greater than about 1 in a blood sample taken at about 30 minutes to about 4 hours indicates the presence of Aβ amyloidosis. In different embodiments, a ratio of relative labeling of Aβ40 to total Aβ greater than about 1 in a blood sample taken at about 1 to about 4 hours indicates the presence of Aβ amyloidosis. In still different embodiments, a ratio of relative labeling of Aβ40 to total Aβ greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ40 to total Aβ greater than about 1 in a blood sample taken at about 1 minutes to about 3 hours indicates the presence of Aβ amyloidosis. In still additional embodiments, a ratio of relative labeling of Aβ40 to total Aβ greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ40 to total Aβ greater than about 1 in a blood sample taken at about 1.5 to about 3 hours indicates the presence of Aβ amyloidosis. In alternative embodiments, a ratio of relative labeling of Aβ40 to total Aβ greater than about 1 in a blood sample taken at about 1.5, about 2.0, about 2.5, or about 3.0 indicates the presence of Aβ amyloidosis. In a preferred embodiment, a ratio of relative labeling of Aβ40 to total Aβ greater than about 1 in a blood sample taken at about 3 hours, indicates the presence of Aβ amyloidosis.

In some embodiments, a ratio of relative labeling of Aβ40 to total Aβ may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of A640 to total Aβ may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of A640 to total Aβ may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of A640 to total Aβ may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of A640 to total Aβ may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of A640 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 minute indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 1 minute indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 10 minutes indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 10 minutes indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 0.5 hour indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 0.5 hour indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 hour indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 1 hour indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1.5 hours indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 1.5 hours indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 2 hours indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 2 hours indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 4 hours indicates the presence of Aβ amyloidosis. In yet other embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 4 hours indicates the presence of Aβ amyloidosis. In additional embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 4 hours indicates the presence of Aβ amyloidosis. In other embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 4 hours indicates the presence of Aβ amyloidosis.

In some preferred embodiments, a ratio of relative labeling of Aβ40 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 3 hours indicates the presence of Aβ amyloidosis. In other preferred embodiments, a ratio of relative labeling of Aβ40 to total Aβ may about 1.20, about 1.21, about 1.22, about 1.23, about 1.24, about 1.25, about 1.26, about 1.27, about 1.28, about 1.29, about 1.30 in a blood sample taken at about 3 hours indicates the presence of Aβ amyloidosis. In still other preferred embodiments, a ratio of relative labeling of Aβ40 to total Aβ may be about 1.2, about 1.3, about 1.4, about 1.5, about 1.6, about 1.7, about 1.8, about 1.9, or about 2.0 in a blood sample taken at about 3 hours indicates the presence of Aβ amyloidosis. In additional preferred embodiments, a ratio of relative labeling of Aβ40 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

In some embodiments, a ratio of relative labeling of Aβ40 to total Aβ less than about 1 in a blood sample taken at about 4 to about 24 hours indicates the presence of Aβ amyloidosis. In other embodiments, a ratio of relative labeling of Aβ40 to total Aβ less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ40 to total Aβ less than about 1 in a blood sample taken at about 9 to about 24 hours indicates the presence of Aβ amyloidosis. In other embodiments, a ratio of relative labeling of Aβ40 to total Aβ less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ40 to total Aβ less than about 1 in a blood sample taken at about 12 to about 24 hours indicates the presence of Aβ amyloidosis. In further embodiments, a ratio of relative labeling of Aβ40 to total Aβ less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ40 to total Aβ less than about 1 in a blood sample taken at about 16 to about 24 hours indicates the presence of Aβ amyloidosis. In still other embodiments, a ratio of relative labeling of Aβ40 to total Aβ less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ40 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ40 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ40 to total Aβ may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ40 to total Aβ less than about 1 in a blood sample taken at about 6 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ40 to total Aβ less than about 1 in a blood sample taken at about 9 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ40 to total Aβ less than about 1 in a blood sample taken at about 12 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ40 to total Aβ less than about 1 in a blood sample taken at about 16 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ40 to total Aβ less than about 1 in a blood sample taken at about 20 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ40 to total Aβ less than about 1 in a blood sample taken at about 24 hours indicates the presence of Aβ amyloidosis. In one embodiment, a ratio of relative labeling of Aβ40 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ40 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ40 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ40 to total Aβ less than about 1 in a blood sample taken at about 9 hours indicates the presence of Aβ amyloidosis. In another preferred embodiment, a ratio of relative labeling of Aβ40 to total Aβ may be about 0.85, 0.80, 0.75, 0.70, 0.65, 0.60, 0.55, or about 0.50 in a blood sample taken at about 9 hours, indicating the presence of Aβ amyloidosis. In still another preferred embodiment, a ratio of relative labeling of Aβ40 to total Aβ less than about 1 in a blood sample taken at about 24 hours indicates the presence of Aβ amyloidosis. In yet another preferred embodiment, a ratio of relative labeling of Aβ40 to total Aβ may be about 0.85, 0.80, 0.75, 0.70, 0.65, 0.60, 0.55, or about 0.50 in a blood sample taken at about 24 hours, indicating the presence of Aβ amyloidosis.

### (vi) Relative Labeling of Aβ38 to Total Aβ

In some embodiments, a ratio of relative labeling of Aβ42 to total Aβ greater than about 1 in a blood sample taken at about 1 minute to about 4 hours indicates the presence of Aβ amyloidosis. In other embodiments, a ratio of relative labeling of Aβ42 to total Aβ greater than about 1 in a blood sample taken at about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 minutes indicates the presence of Aβ amyloidosis. In other embodiments, a ratio of relative labeling of Aβ42 to total Aβ greater than about 1 in a blood sample taken at about 0.5, about 1.0, about 1.5, about 2.0, about 2.5, about 3.0, about 3.5, or about 4.0 hours indicates the presence of Aβ amyloidosis. In different embodiments, a ratio of relative labeling of Aβ42 to total Aβ greater than about 1 in a blood sample taken at about 10 minutes to about 4 hours indicates the presence of Aβ amyloidosis. In different embodiments, a ratio of relative labeling of Aβ42 to total Aβ greater than about 1 in a blood sample taken at about 30 minutes to about 4 hours indicates the presence of Aβ amyloidosis. In different embodiments, a ratio of relative labeling of Aβ42 to total Aβ greater than about 1 in a blood sample taken at about 1 to about 4 hours indicates the presence of Aβ amyloidosis. In still different embodiments, a ratio of relative labeling of Aβ42 to total Aβ greater than about 1 in a blood sample taken at about 1.0, about 1.5, about 2.0, about 2.5, about 3.0, about 3.5, or about 4.0 hours indicates the presence of Aβ amyloidosis. In additional embodiments, a ratio of relative labeling of Aβ42 to Total Aβ greater than about 1 in a blood sample taken at about 1 minutes to about 3 hours indicates the presence of Aβ amyloidosis. In still additional embodiments, a ratio of relative labeling of Aβ42 to total Aβ greater than about 1 in a blood sample taken at about 1.0 minute, about 10 minutes, about 30 minutes, about 1 hour, about 1.5 hours, about 2.0 hours, about 2.5 hours, or about 3.0 hours indicates the presence of Aβ amyloidosis. In further embodiments, a ratio of relative labeling of Aβ42 to total Aβ greater than about 1 in a blood sample taken at about 1.5 to about 3 hours indicates the presence of Aβ amyloidosis. In alternative embodiments, a ratio of relative labeling of Aβ42 to total Aβ greater than about 1 in a blood sample taken at about 1.5, about 2.0, about 2.5, or about 3.0 indicates the presence of Aβ amyloidosis. In a preferred embodiment, a ratio of relative labeling of Aβ42 to total Aβ greater than about 1 in a blood sample taken at about 3 hours, indicates the presence of Aβ amyloidosis.

In some embodiments, a ratio of relative labeling of Aβ38 to total Aβ may be about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis. In other embodiments, a ratio of relative labeling of A638 to total Aβ may be about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, or about 1.50 in a blood sample taken at about 0.5 to about 4 hours, indicating the presence of Aβ amyloidosis. In still other embodiments, a ratio of relative labeling of A638 to total Aβ may be about 1.15, about 1.16, about 1.17, about 1.18, about 1.19, about 1.20, about 1.21, about 1.22, about 1.23, about 1.24, or about 1.25 in a blood sample taken at about 0.5 to about 4 hours, indicating the presence of Aβ amyloidosis. In yet other embodiments, a ratio of relative labeling of A638 to total Aβ may about 1.20, about 1.21, about 1.22, about 1.23, about 1.24, about 1.25, about 1.26, about 1.27, about 1.28, about 1.29, about 1.30 in a blood sample taken at about 0.5 to about 4 hours, indicating the presence of Aβ amyloidosis. In additional embodiments, a ratio of relative labeling of A638 to total Aβ may be about 1.2, about 1.3, about 1.4, about 1.5, about 1.6, about 1.7, about 1.8, about 1.9, or about 2.0 in a blood sample taken at about 0.5 to about 4 hours, indicating the presence of Aβ amyloidosis. In other embodiments, a ratio of relative labeling of A638 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 to about 4 hours, indicating the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 minute indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 1 minute indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 1 minute indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 minute indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 10 minutes indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 10 minutes indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 10 minutes indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 10 minutes indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 0.5 hour indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 0.5 hour indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 0.5 hour indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 0.5 hour indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1 hour indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 1 hour indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 1 hour indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1 hour indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 1.5 hours indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 1.5 hours indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 1.5 hours indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 1.5 hours indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 2 hours indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 2 hours indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 2 hours indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 2 hours indicates the presence of Aβ amyloidosis.

In some embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 4 hours indicates the presence of Aβ amyloidosis. In yet other embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 4 hours indicates the presence of Aβ amyloidosis. In additional embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 4 hours indicates the presence of Aβ amyloidosis. In other embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 4 hours indicates the presence of Aβ amyloidosis.

In some preferred embodiments, a ratio of relative labeling of Aβ38 to total Aβ of about 1.10, about 1.15, about 1.16, about 1.17, about 1.18, about 1.19, about 1.20, about 1.21, about 1.22, about 1.23, about 1.24, about 1.25 or above in a blood sample taken at about 3 hours indicates the presence of Aβ amyloidosis. In other preferred embodiments, a ratio of relative labeling of Aβ38 to total Aβ may about 1.20, about 1.21, about 1.22, about 1.23, about 1.24, about 1.25, about 1.26, about 1.27, about 1.28, about 1.29, about 1.30 in a blood sample taken at about 3 hours indicates the presence of Aβ amyloidosis. In still other preferred embodiments, a ratio of relative labeling of Aβ38 to total Aβ may be about 1.2, about 1.3, about 1.4, about 1.5, about 1.6, about 1.7, about 1.8, about 1.9, or about 2.0 in a blood sample taken at about 3 hours indicates the presence of Aβ amyloidosis. In additional preferred embodiments, a ratio of relative labeling of Aβ38 to total Aβ may be about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above in a blood sample taken at about 3 hours indicates the presence of Aβ amyloidosis.

In some embodiments, a ratio of relative labeling of Aβ38 to total Aβ less than about 1 in a blood sample taken at about 4 to about 24 hours indicates the presence of Aβ amyloidosis. In other embodiments, a ratio of relative labeling of Aβ38 to total Aβ less than about 1 in a blood sample taken at about 4, about 5, about 6, about 7, about 8, about 9, about 10, about 11, about 12, about 13, about 14, about 15, about 16, about 17, about 18, about 19, about 20, about 21, about 22, about 23, or about 24 hours indicates the presence of Aβ amyloidosis. In different embodiments, a ratio of relative labeling of Aβ38 to total Aβ less than about 1 in a blood sample taken at about 9 to about 24 hours indicates the presence of Aβ amyloidosis. In other embodiments, a ratio of relative labeling of Aβ38 to total Aβ less than about 1 in a blood sample taken at about 9, about 10, about 11, about 12, about 13, about 14, about 15, about 16, about 17, about 18, about 19, about 20, about 21, about 22, about 23, or about 24 hours indicates the presence of Aβ amyloidosis. In alternative embodiments, a ratio of relative labeling of Aβ38 to total Aβ less than about 1 in a blood sample taken at about 12 to about 24 hours indicates the presence of Aβ amyloidosis. In further embodiments, a ratio of relative labeling of Aβ38 to total Aβ less than about 1 in a blood sample taken at about 12, about 13, about 14, about 15, about 16, about 17, about 18, about 19, about 20, about 21, about 22, about 23, or about 24 hours indicates the presence of Aβ amyloidosis. In other embodiments, a ratio of relative labeling of Aβ38 to total Aβ less than about 1 in a blood sample taken at about 16 to about 24 hours indicates the presence of Aβ amyloidosis. In still other embodiments, a ratio of relative labeling of Aβ38 to total Aβ less than about 1 in a blood sample taken at about 16, about 17, about 18, about 19, about 20, about 21, about 22, about 23, or about 24 hours indicates the presence of Aβ amyloidosis.

In some embodiments, a ratio of relative labeling of Aβ38 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50, about 0.49, about 0.48, about 0.47, about 0.46, about 0.45, about 0.44, about 0.43, about 0.42, about 0.41, about 0.40, about 0.39, about 0.38, about 0.37, about 0.36, about 0.35, about 0.34, about 0.33, about 0.32, about 0.31, about 0.30, about 0.29, about 0.28, about 0.27, about 0.26, about 0.25, about 0.24, about 0.23, about 0.22, about 0.21, about 0.20, about 0.10, or less in a blood sample taken at about 4 to about 24 hours, indicating the presence of Aβ amyloidosis. In other embodiments, a ratio of relative labeling of Aβ38 to total Aβ may be about 0.90, about 0.89, about 0.88, about 0.87, about 0.86, about 0.85, about 0.84, about 0.83, about 0.82, about 0.81, about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.60, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis. In other embodiments, a ratio of relative labeling of Aβ38 to total Aβ may be about 0.80, about 0.79, about 0.78, about 0.77, about 0.76, about 0.75, about 0.74, about 0.73, about 0.72, about 0.71, about 0.70, about 0.69, about 0.68, about 0.67, about 0.66, about 0.65, about 0.64, about 0.63, about 0.62, about 0.61, about 0.6, about 0.59, about 0.58, about 0.57, about 0.56, about 0.55, about 0.54, about 0.53, about 0.52, about 0.51, about 0.50 or less in a blood sample taken at about 4 to about 24 hours, indicating the presence of Aβ amyloidosis.

In some embodiments, a ratio of relative labeling of Aβ38 to total Aβ less than about 1 in a blood sample taken at about 6 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 6 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 6 hours, indicating the presence of Aβ amyloidosis.

In some embodiments, a ratio of relative labeling of Aβ38 to total Aβ less than about 1 in a blood sample taken at about 9 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 9 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 9 hours, indicating the presence of Aβ amyloidosis.

In some embodiments, a ratio of relative labeling of Aβ38 to total Aβ less than about 1 in a blood sample taken at about 12 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 12 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 12 hours, indicating the presence of Aβ amyloidosis.

In some embodiments, a ratio of relative labeling of Aβ38 to total Aβ less than about 1 in a blood sample taken at about 16 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 16 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 16 hours, indicating the presence of Aβ amyloidosis.

In some embodiments, a ratio of relative labeling of Aβ38 to total Aβ less than about 1 in a blood sample taken at about 20 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 20 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 20 hours, indicating the presence of Aβ amyloidosis.

In some embodiments, a ratio of relative labeling of Aβ38 to total Aβ less than about 1 in a blood sample taken at about 24 hours indicates the presence of Aβ amyloidosis. In one embodiment, a ratio of relative labeling of Aβ38 to total Aβ may be about 0.9, 0.89, 0.88, 0.87, 0.86, 0.85, 0.84, 0.83, 0.82, 0.81, 0.8, 0.79, 0.78, 0.77, 0.76, 0.75, 0.74, 0.73, 0.72, 0.71, 0.7, 0.69, 0.68, 0.67, 0.66, 0.65, 0.64, 0.63, 0.62, 0.61, 0.6, 0.59, 0.58, 0.57, 0.56, 0.55, 0.54, 0.53, 0.52, 0.51, 0.5, 0.49, 0.48, 0.47, 0.46, 0.45, 0.44, 0.43, 0.42, 0.41, 0.4, 0.39, 0.38, 0.37, 0.36, 0.35, 0.34, 0.33, 0.32, 0.31, 0.3, 0.29, 0.28, 0.27, 0.26, 0.25, 0.24, 0.23, 0.22, 0.21, 0.2, 0.1, or less in a blood sample taken at about 24 hours, indicating the presence of Aβ amyloidosis. In another embodiment, a ratio of relative labeling of Aβ38 to total Aβ may be about 0.9, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis. In another embodiment, a ratio of relative labeling of Aβ38 to total Aβ may be about 0.8, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51, 0.50 or less in a blood sample taken at about 24 hours, indicating the presence of Aβ amyloidosis.

In a preferred embodiment, a ratio of relative labeling of Aβ38 to total Aβ less than about 1 in a blood sample taken at about 9 hours indicates the presence of Aβ amyloidosis. In another preferred embodiment, a ratio of relative labeling of Aβ38 to total Aβ may be about 0.85, 0.80, 0.75, 0.70, 0.65, 0.60, 0.55, or about 0.50 in a blood sample taken at about 9 hours, indicating the presence of Aβ amyloidosis. In still another preferred embodiment, a ratio of relative labeling of Aβ38 to total Aβ less than about 1 in a blood sample taken at about 24 hours indicates the presence of Aβ amyloidosis. In yet another preferred embodiment, a ratio of relative labeling of Aβ38 to total Aβ may be about 0.85, 0.80, 0.75, 0.70, 0.65, 0.60, 0.55, or about 0.50 in a blood sample taken at about 24 hours, indicating the presence of Aβ amyloidosis.

### (vii) Exemplary Embodiments

In one exemplary embodiment, the in vivo relative labeling of Aβ42 and Aβ40 are measured by administering labeled leucine to a subject and collecting one or more blood samples over a period of 24 hours. The labeled leucine may be administered as an intravenous bolus, an intravenous infusion, or an oral bolus. The amount of labeled and unlabeled Aβ in the biological samples is typically determined by immunoprecipitation followed by LC-ESI-tandem MS. From these measurements, a ratio of labeled to unlabeled Aβ may be calculated to determine the relative labeling of Aβ42 and Aβ40. A ratio of relative labeling of Aβ42 to relative labeling of Aβ40 may then be calculated in a given sample. A ratio of relative labeling of Aβ42 to relative labeling of Aβ40 in a given sample other than 1 indicates the presence of Aβ amyloidosis.

In another exemplary embodiment, the in vivo relative labeling of Aβ42 and Aβ38 are measured by administering labeled leucine to a subject and collecting one or more blood samples over a period of 24 hours. The labeled leucine may be administered as an intravenous bolus, an intravenous infusion, or an oral bolus. The amount of labeled and unlabeled Aβ in the biological samples is typically determined by immunoprecipitation followed by LC-ESI-tandem MS. From these measurements, a ratio of labeled to unlabeled Aβ may be calculated to determine the relative labeling of Aβ42 and Aβ38. A ratio of relative labeling of Aβ42 to relative labeling of Aβ38 may then be calculated in a given sample. A ratio of relative labeling of Aβ42 to relative labeling of Aβ38 in a given sample other than 1 indicates the presence of Aβ amyloidosis.

In another exemplary embodiment, the in vivo relative labeling of Aβ42 and total Aβ are measured by administering labeled leucine to a subject and collecting one or more blood samples over a period of 24 hours. The labeled leucine may be administered as an intravenous bolus, an intravenous infusion, or an oral bolus. The amount of labeled and unlabeled Aβ in the biological samples is typically determined by immunoprecipitation followed by LC-ESI-tandem MS. From these measurements, a ratio of labeled to unlabeled Aβ may be calculated to determine the relative labeling of Aβ42 and Aβ38. A ratio of relative labeling of Aβ42 to relative labeling of Aβ38 may then be calculated in a given sample. A ratio of relative labeling of Aβ42 to relative labeling of Aβ38 in a given sample other than 1 indicates the presence of Aβ amyloidosis.

### (c) Aβ Peak Time

In another aspect, the present disclosure encompasses a method for detecting Aβ amyloidosis in a subject, the method comprising measuring the in vivo relative labeling of Aβ42 and another Aβ variant in at least two biological samples obtained from the subject; determining the peak time for labeled Aβ42 and the other labeled Aβ variant; and calculating the ratio of the Aβ42 peak time to the peak time of the other Aβ variant; wherein a ratio of less than about 1 indicates the presence of Aβ amyloidosis. Generally speaking, the peak time of a labeled Aβ variant can be determined from at least 2, 3, 4, 5, 6, 7, 8, 9, 10 or more measurements of the in vivo levels of Aβ, wherein the measurements of the in vivo levels of Aβ are determined as described in Section I(a). In preferred embodiments, a biological sample is a blood sample or a CSF sample, and the other Aβ variant is selected from the group consisting of Aβ40, Aβ38 or total Aβ. The two or more biological samples generally include at least one sample obtained during Aβ labeling increase and at least one sample obtained during Aβ labeling decrease. Suitable time points are described in Section I(a).

In certain embodiments, the labeled moiety is administered as a bolus, and two or more biological samples may be obtained from the subject at about 1 min to about 12 hours, at about 1 min to about 10 hours, at about 1 min to about 8 hours, at about 1 min to about 6 hours, or at about 1 min to about 5 hours. In other embodiments, the labeled moiety is administered as a bolus, and two or more biological samples may be obtained from the subject at about 15 min to about 12 hours, at about 15 min to about 10 hours, at about 15 min to about 8 hours, at about 15 min to about 6 hours, or at about 15 min to about 5 hours. In still other embodiments, the labeled moiety is administered as a bolus, and two or more biological samples may be obtained from the subject at about 30 min to about 12 hours, at about 30 min to about 10 hours, at about 30 min to about 8 hours, at about 30 min to about 6 hours, or at about 30 min to about 5 hours. In yet other embodiments, the labeled moiety is administered as a bolus, and two or more biological samples may be obtained from the subject at about 1 hour to about 12 hours, at about 1 hour to about 10 hours, at about 1 hour to about 8 hours, at about 1 hour to about 6 hours or about 1 hour to about 5 hours. Alternatively, the labeled moiety is administered as a bolus, and two or more biological samples may be obtained from the subject at at about 2 hours to about 12 hours, at about 2 hours to about 10 hours, at about 2 hours to about 8 hours, at about 2 hours to about 6 hours or about 2 hours to about 5 hours.

The labeled moiety may also be administered as an infusion. The length of an infusion can and will affect sampling time as described in Section I(a). In some embodiments, the labeled moiety is administered as about a 9-hour infusion, and two or more biological samples may be obtained from the subject at about 6 hours to about 32 hours, at about 6 hours to about 30 hours, at about 6 hours to about 28 hours, at about 6 hours to about 26 hours, at about 6 hours to about 24 hours, at about 6 hours to about 22 hours, or at about 6 hours to about 20 hours. In other embodiments, the labeled moiety is administered as about a 9-hour infusion and two or more blood samples may be obtained from the subject at about 6 hours to about 12 hours, at about 6 hours to about 11 hours, or at about 6 hours to about 10 hours. In still other embodiments, the labeled moiety is administered as about a 9-hour infusion and two or more blood samples may be obtained from the subject at about 7 hours to about 12 hours, at about 7 hours to about 11 hours, or at about 7 hours to about 10 hours. In yet other embodiments, the labeled moiety is administered as about a 9-hour infusion and two or more CSF samples may be obtained from the subject at about 14 hours to about 32 hours, at about 14 hours to about 30 hours, at about 14 hours to about 28 hours, at about 14 hours to about 26 hours, at about 14 hours to about 24 hours, at about 14 hours to about 22 hours, or at about 14 hours to about 20 hours. In yet other embodiments, the labeled moiety is administered as about a 9-hour infusion and two or more CSF samples may be obtained from the subject at about 16 hours to about 32 hours, at about 16 hours to about 30 hours, at about 16 hours to about 28 hours, at about 16 hours to about 26 hours, at about 16 hours to about 24 hours, at about 16 hours to about 22 hours, or at about 16 hours to about 20 hours.

In another aspect, the present disclosure encompasses a method for monitoring the effectiveness of a therapeutic and/or treatment regimen, the method comprising measuring the in vivo relative labeling of Aβ42 and another Aβ variant in at least two biological samples obtained from the subject; determining the peak time for labeled Aβ42 and the other labeled Aβ variant; and calculating the ratio of the Aβ42 peak time to the peak time of the other Aβ variant before and during and/or after treatment, wherein a decrease in the value of the kinetic parameter, by an acceptable degree of statistical significance, indicates the treatment is effective, and no change or an increase in the kinetic parameter indicates the treatment is not effective. Suitable times for obtaining a sample are as described above in this Section.

### (d) Use of Model-Based Parameters

One or more kinetic parameters derived from a compartmental model of Aβ turnover kinetics may be used to detect Aβ amyloidosis, to monitor the progression of Aβ amyloidosis, or to evaluate whether a new therapy or treatment regimen alters Aβ amyloidosis. Suitable methods for developing a compartmental model of Aβ turnover kinetics are known in the art; see for example Potter et al. Sci Transl Med 5(189), 2013 and WO 2014081851, each hereby incorporated by reference in its entirety. Suitable methods are also further detailed in the Examples. Generally speaking, a compartmental model of Aβ turnover kinetics can be derived from 1, 2, 3, 4, 5, 6, 7, 8, 9, 10 or more measurements of the in vivo levels of Aβ, wherein the measurements of the in vivo levels of Aβ are determined as described in Section I(a). In preferred embodiments, the measurements of the in vivo levels of Aβ are determined from a CSF or blood sample obtained from a subject intravenously or orally administered a labeled moiety. In particularly preferred embodiments, the labeled moiety is ¹³C₆-leucine. Non-limiting examples of suitable parameters of a compartmental model of Aβ turnover kinetics includes fractional turnover rate (FTR) for the irreversible loss of each Aβ variant, a ratio of Aβ42 FTR to the FTR of another Aβ variant, kₑₓ₃₈, kₑₓ₄₀, kₑₓ₄₂, k_{delay}, k_{APP}, k_{Aβ} for each Aβ variant (“k_{Aβxx}”). In preferred embodiments, a kinetic parameter is selected from the group consisting of Aβ38 FTR, Aβ40 FTR, Aβ42 FTR, Aβ total FTR, ratio of Aβ42 FTR to Aβ40 FTR, a ratio of Aβ42 FTR to Aβ28 FTR, Aβ42 FTR to total Aβ FTR, kₑₓ₄₂, and a combination thereof.

In an exemplary embodiment, a model comprises a plasma leucine pool, a subsystem for production of Aβ through APP and C99, turnover of Aβ, and transport of Aβ to CSF. Fluid transport through the CSF may be depicted as a system of multiple compartments (e.g. 2, 3, 4, 5, or more compartments), which together with the APP and C99 compartment represent the total number of compartments that comprise a time delay that is common to all Aβ isoforms, which can be resolved from the turnover of Aβ. In certain embodiments, fluid transport through the CSF may be depicted as a system of three compartments. The model may allow for exchange of Aβ38, Aβ40, and/or Aβ42 with another compartment, In certain embodiments, the model may only allow for exchange of Aβ42 with another compartment. Non-limiting examples of parameters obtained from kinetic analysis of Aβ using the model may include, but are not limited to, fractional turnover rate (FTR) for the irreversible loss of each Aβ variant, kₑₓ₃₈, kₑₓ₄₀, kₑₓ₄₂, k_{delay}, k_{APP}, k_{Aβ} for each Aβ variant (“k_{Aβxx}”). In preferred embodiments, a kinetic parameter is selected from the group consisting of Aβ38 FTR, Aβ40 FTR, Aβ42 FTR, Aβ total FTR, ratio of Aβ42 FTR to Aβ40 FTR, a ratio of Aβ42 FTR to Aβ28 FTR, Aβ42 FTR to total Aβ FTR, kₑₓ₄₂, and a combination thereof.

In some aspects, a method for detecting Aβ amyloidosis in a subject may comprise calculating one or more kinetic parameter derived from a compartmental model of Aβ turnover kinetics in a subject over time. As noted in the Examples, amyloidosis is associated with a higher irreversible loss of soluble Aβ42 and a higher reversible exchange rate that is independent of the age-associated slowing of Aβ turnover that affects all Aβ isoforms. In some embodiments, the kinetic parameter is Aβ42 FTR and an increase in Aβ42 FTR over time indicates Aβ amyloidosis when the increase is greater than would have been predicted by age-alone. For example, the age-associated increase in Aβ turnover rates is about two-fold slowing over three decades. Accordingly, if Aβ42 FTR in a subject increases more than about two-fold over three decades (i.e. as predicted by age alone), then the change in FTR can be attributed to amyloid-associated alterations. A skilled artisan will appreciate that the amount of the increase can and will vary depending upon several factors, including frequency of measurement. Generally speaking, amyloidosis may be associated with an increase of about 5%, preferably about 10%, more preferably about 20%, more preferably about 30% or more over the age-associated increase in Aβ42 FTR. In other embodiments, rather than measuring Aβ42 FTR in a subject over time, a subject's Aβ42 FTR is compared with the Aβ42 FTR of an age-matched control subject without amyloidosis or, more preferably, a group of age-matched control subjects without amyloidosis. In these embodiments, amyloidosis may be associated with an increase of about 5%, 10%, 15%, 20%, 25%, 30%, 35%, 40%, 45%, or about 50% or more increase in Aβ42 FTR. In other embodiments, the kinetic parameter is Aβ42 exchange rate (kₑₓ₄₂) and an increase in kₑₓ₄₂ over time indicates Aβ amyloidosis. Without wishing to be bound by theory, Applicants believe kₑₓ₄₂ is independent of age. Accordingly, an increase kₑₓ₄₂ in a subject over time from zero to greater than zero indicates the presence of Aβ amyloidosis. For example, an Aβ42 exchange rate of greater than about 0.01, 0.02, or 0.03 indicates the presence of Aβ amyloidosis.

In some aspects, a method for detecting Aβ amyloidosis in a subject may comprise calculating the ratio of Aβ42 FTR to Aβxx FTR, wherein Aβxx is an Aβ variant other than Aβ42. As stated elsewhere, Aβ42 kinetics are altered compared to other Aβ variants in subjects with amyloidosis. Accordingly, comparing Aβ4242 FTR to Aβₓₓ FTR allows one of skill in the art to detect Aβ amyloidosis without profiling Aβ kinetics over time. Aβxx may be selected from the group consisting of Aβ38, Aβ40 and total Aβ. Generally, a ratio of greater than about 1 indicates the presence of Aβ amyloidosis. In some embodiments a ratio of about 1.10, about 1.15, about 1.16, about 1.17, about 1.18, about 1.19, about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, about 1.60, about 1.61, about 1.62, about 1.63, about 1.64, about 1.65, about 1.66, about 1.67, about 1.68, about 1.69, about 1.70, about 1.71, about 1.72, about 1.73, about 1.74, about 1.75, about 1.76, about 1.77, about 1.78, about 1.79, about 1.80, about 1.81, about 1.82, about 1.83, about 1.84, about 1.85, about 1.86, about 1.87, about 1.88, about 1.89, about 1.90, about 1.91, about 1.92, about 1.93, about 1.94, about 1.95, about 1.96, about 1.97, about 1.98, about 1.99, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0 or above indicates the presence of amyloidosis. In other embodiments, a subject's Aβ42 FTR to Aβxx FTR ratio is compared with the same ratio of an age-matched control subject without amyloidosis or, more preferably, a group of age-matched control subjects without amyloidosis. In these embodiments, amyloidosis may be associated with an increase of about 1%, 5%, 10%, 15%, 20%, 25%, 30%, 35%, 40%, 45%, or about 50% or more increase in Aβ42 FTR.

In some aspects, a method for detecting Aβ amyloidosis in a subject may comprise calculating the Aβ42 exchange rate (kₑₓ₄₂). Generally an Aβ42 exchange rate of greater than about 0.01, preferably greater than about 0.02, more preferably greater than about 0.03 indicates the presence of Aβ amyloidosis. In some embodiments an Aβ42 exchange rate about 0.01, 0.02, 0.03, 0.04, 0.05 or more indicates Aβ amyloidosis.

In some aspects, a method for monitoring the effectiveness of a therapeutic and/or treatment regimen comprises calculating one or more kinetic parameter derived from a compartmental model of Aβ turnover kinetics in a subject before and/or during and/or after treatment, wherein the kinetic parameter is selected from the group consisting of Aβ42 FTR, a ratio of Aβ42 FTR to the FTR of another Aβ variant (Aβₓₓ FTR), and Aβ42 exchange rate, and wherein a decrease in the value of the kinetic parameter, by an acceptable degree of statistical significance, indicates the treatment is effective, and no change or an increase in the kinetic parameter indicates the treatment is not effective. An acceptable degree of statistical significance is known to one skilled in the art.

### (e) Optionally Comparing to a Control

In some embodiments, comparing the level of an Aβ variant, the relative labeling of Aβ42, the ratio of Aβ42 relative labeling to the relative labeling of another Aβ variant (Aβxx), Aβ42 FTR, the ratio of Aβ42 FTR to Aβxx FTR, or Aβ42 exchange rate a subject to the same measurement in an individual with no amyloidosis, one skilled in the art may be able to diagnose Aβ amyloidosis in a subject before the development of symptoms associated with Aβ amyloidosis. In other embodiments, by comparing the level of an Aβ variant in a subject to the level of another Aβ variant in the subject, one skilled in the art may be able to diagnose Aβ amyloidosis in the subject before the development of symptoms associated with Aβ amyloidosis. In addition, the invention permits the measurement of the pharmacodynamic effects of disease-modifying therapeutics in a subject.

### II. Kits for Detecting, Diagnosing or Monitoring the Progression or Treatment of Aβ Amyloidosis

The current invention provides kits for diagnosing or monitoring the progression or treatment of Aβ amyloidosis by measuring the ratio of relative labeling of two Aβ variants in a subject. Generally, a kit comprises a labeled amino acid, means for administering the labeled amino acid, means for collecting one or more blood samples over time, and instructions for detecting and determining the ratio of labeled to unlabeled protein so that the ratio of relative labeling of the two Aβ variants may be calculated. A ratio of relative labeling of the two Aβ variants of about one indicates the absence of Aβ amyloidosis, whereas a ratio of relative labeling of the two Aβ variants other than one indicates the absence of Aβ amyloidosis. These comparisons may enable a practitioner to identify a subject at risk of developing diseases associated with Aβ amyloidosis, predict the advent of diseases associated with Aβ amyloidosis, monitor the progression of Aβ amyloidosis, or verify the effectiveness of a treatment for Aβ amyloidosis. In a preferred embodiment, the kit comprises ¹³C₆-leucine or ¹³C₆-phenylalanine, the Aβ variants to be measured are Aβ42 and Aβ40, and the disease associated with Aβ is AD.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Aβ variant”, as used herein, refers to any naturally occurring Aβ isoform known in the art. The length of each Aβ isoform is defined by the N-terminal and C-terminal cleavage sites. Non-limiting examples of C-terminal cleavage sites known in the art include position 43, 42, 41, 40, 39, 38, 37, and 35. Non-limiting examples of N-terminal cleavage sites known in the art include position 1, 2, and 5. Thus, the term “Aβ variant” refers to any combination of N-terminal and C-terminal cleavage. For example, the term “Aβ variant” may refer to Aβ1-35, Aβ1-37, Aβ1-38, Aβ1-39, Aβ1-40, Aβ1-41, Aβ1-42, Aβ1-43, Aβ2-35, Aβ2-37, Aβ2-38, Aβ2-39, Aβ2-40, Aβ2-41, Aβ2-42, Aβ2-43, Aβ5-35, Aβ5-37, Aβ5-38, Aβ5-39, Aβ5-40, Aβ5-41, Aβ5-42, or Aβ5-43. Also included in the definition of the term “Aβ variant” are C-truncated forms of Aβ. Non-limiting examples of C-truncated forms of Aβ include Aβ1-14, Aβ1-15, Aβ1-16, Aβ1-17, Aβ2-14, Aβ2-15, Aβ2-16, and Aβ2-17.

“Aβ38”, as used herein, refers to Aβx-38, where “x” is any N-terminal cleavage site.

“Aβ40”, as used herein, refers to Aβx-40, where “x” is any N-terminal cleavage site.

“Aβ42”, as used herein, refers to Aβx-42, where “x” is any N-terminal cleavage site.

“Aβ43”, as used herein, refers to Aβx-43, where “x” is any N-terminal cleavage site. The amino acid sequence of Aβ1-43 is DAEFRHDSGYEVHHQKLVFFAEDVGSNKGAIIGLMVGGVVIAT (SEQ ID NO: 1).

“Clearance phase”, as used herein, may be used interchangeably with the phrase “decreasing phase of the labeling”, and refers to a state in which labeled amyloid beta is decreasing. The timing of the clearance phase depends, in part, on the length of the labeling phase. For example, after bolus labeling, the clearance phase generally begins at about 3 to about 6 hours. A skilled artisan will appreciate that if an alternative labeling protocol contemplated herein is used, for example an IV infusion for 6 hours, the clearance phase will occur later than at about 6 hours.

“Fractional clearance rate” or FCR is calculated as the natural log of the ratio of labeled biomolecule, such as tau, over a specified period of time.

“Fractional synthesis rate” or FSR is calculated as the slope of the increasing ratio of labeled biomolecule, such as tau, over a specified period of time divided by the predicted value of the labeled precursor.

“Fractional turnover rate” or FTR is the rate of irreversible loss of a biomolecule, such as an Aβ variant, from the CNS, and is the sum of losses to CSF and other loss pathways (e.g. local tissue uptake, proteolysis, deposition into amyloid plaques).

“Isotope” refers to all forms of a given element whose nuclei have the same atomic number but have different mass numbers because they contain different numbers of neutrons. By way of a non-limiting example, ¹²C and ¹³C are both stable isotopes of carbon.

“kₑₓ” or “exchange rate” refers to the rate of entry of an Aβ variant into an exchange compartment in a compartmental-model of Aβ turnover kinetics. “kₑₓ₄₂^{”} refers to the rate of Aβ42 exchange with another compartment, particularly when amyloid plaques are present, which alters the shape of the back end of the tracer time course and causes isotopic dilution of the peak enrichment for Aβ42. Similarly, “kₑₓ₃₈” refers to the rate of Aβ38 exchange and “kₑₓ₄₀” refers to the rate of Aβ40 exchange.

“Lag time” generally refers to the delay of time from when the biomolecule is first labeled until the labeled biomolecule is detected.

“Metabolism” refers to any combination of the synthesis, transport, breakdown, modification, or clearance rate of a biomolecule.

“Metabolic index” refers to a measurement comprising the fractional synthesis rate (FSR) and the fractional clearance rate (FCR) of the biomolecule of interest. Comparison of metabolic indices from normal and diseased individuals may aid in the diagnosis or monitoring of neurological or neurodegenerative diseases.

“Neurally derived cells” includes all cells within the blood-brain-barrier including neurons, astrocytes, microglia, choroid plexus cells, ependymal cells, other glial cells, etc.

“Production phase”, as used herein, may be used interchangeably with the phrases “labeling phase” or “increasing phase of the labeling”, and refers to a state in which labeled amyloid beta is increasing.

“Relative labeling” refers to the ratio of labeled tau to unlabeled tau or the percent labeled tau. Relative labeling may be expressed using any suitable unit. As a non-limiting example, the ratio of labeled tau to unlabeled tau may be expressed as a tracer to trace relationship (TTR) obtained from a mass spectrometric analysis. As another non-limiting example, TTR ratios may be converted to mole fraction labeled.

“Steady state” refers to a state during which there is insignificant change in the measured parameter over a specified period of time.

“Synthesis rate” refers to the rate at which the biomolecule of interest is synthesized.

In metabolic tracer studies, a “stable isotope” is a nonradioactive isotope that is less abundant than the most abundant naturally occurring isotope.

“Subject” as used herein means a living organism having a central nervous system. In particular, the subject is a mammal. Suitable subjects include research animals, companion animals, farm animals, and zoo animals. The preferred subject is a human.

“Total Aβ”, as used herein, refers to any labeled Aβ isoform.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and the scope of the invention. Therefore, all matter set forth or shown in the accompanying drawings is to be interpreted as illustrative and not in a limiting sense.

### EXAMPLES

The following examples are included to demonstrate preferred embodiments of the invention.

### Example 1

### Labeling Protocol Provides a Simple Blood Test of Amyloidosis or AD Risk

18 participants were labeled with 3 labeling protocols (IV bolus, IV 60 minute infusion, or oral bolus) of labeled leucine 800 mg. Two clinical groups, cognitively normal (CDR 0) vs. impaired (CDR 0.5 or 1), were enrolled n=9 per group. Within each group, n=3 per labeling protocol. Blood samples were collected over 24 hours for labeled Abeta isoform measurements and free leucine labeling in blood.

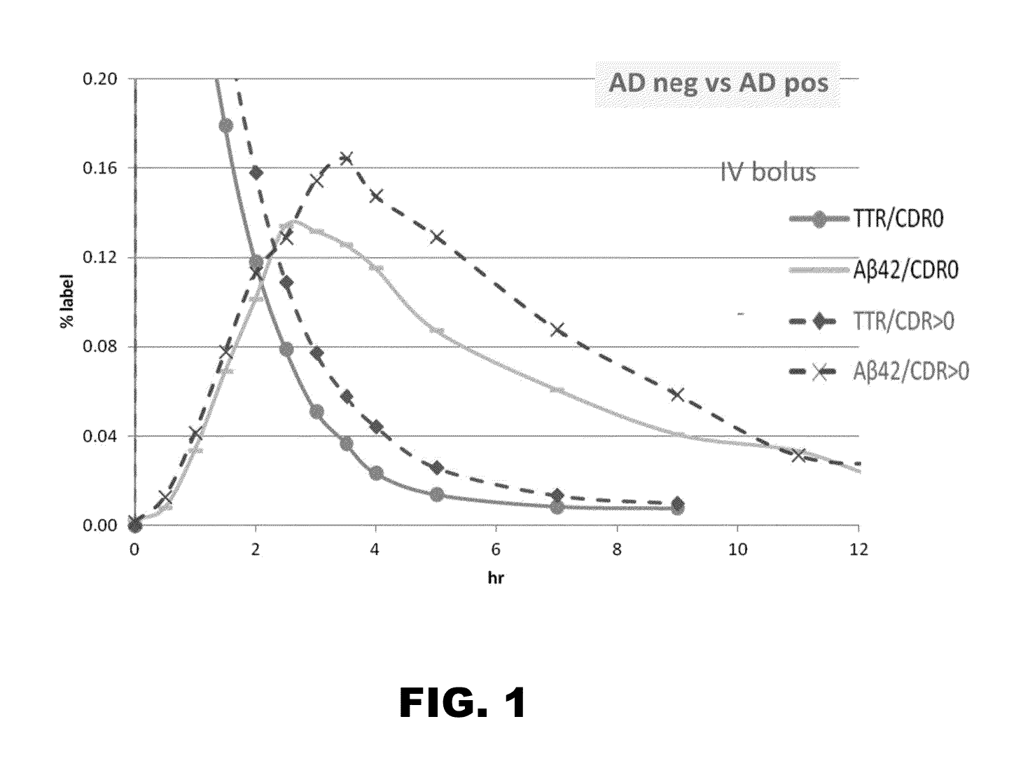

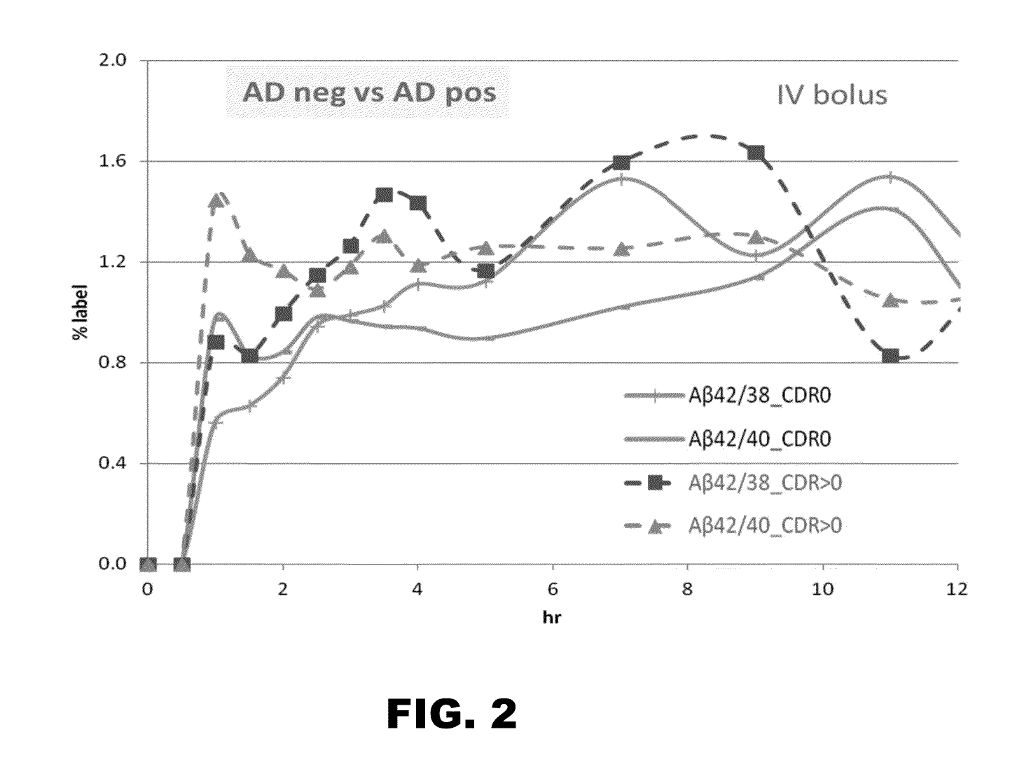

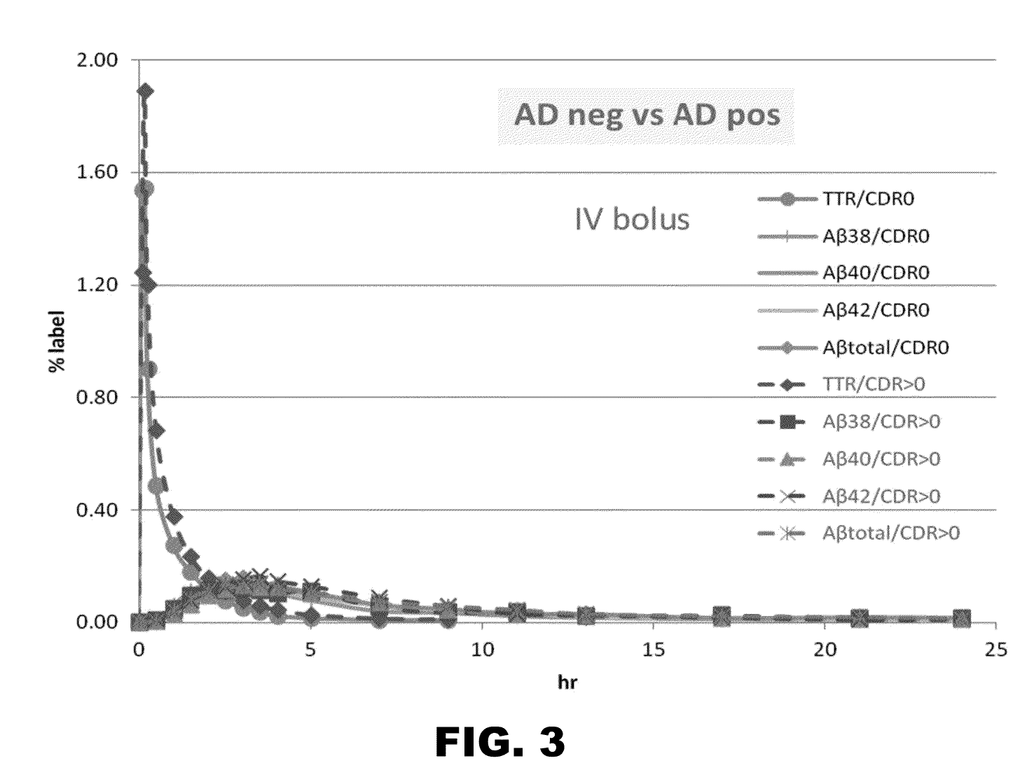

As shown in FIG. 3, leucine labeling was higher in very mild or mild dementia (CDR>0) vs. controls (CDR0). For example, the percent labeled tracer-to-tracee ratio (TTR, measured by quantifying the relative amounts of ¹³C₆-leucine and dividing by the amount of unlabeled leucine in each sample) in CDR>0 plasma samples (blue dashed line) is greater than in CDR) plasma samples (orange solid line). Labeling kinetics are also altered in patients with amyloidosis, as shown in FIG. 1 (Aβ42 ¹³C₆-leucine labeling in plasma samples from amyloid negative vs. amyloid positive patients) and FIG. 2 (Aβ42/40 ratios ¹³C₆-leucine labeling in plasma samples from amyloid negative vs. amyloid positive patients). Amyloid status was determined by PIB/CSF Aβ42 status.

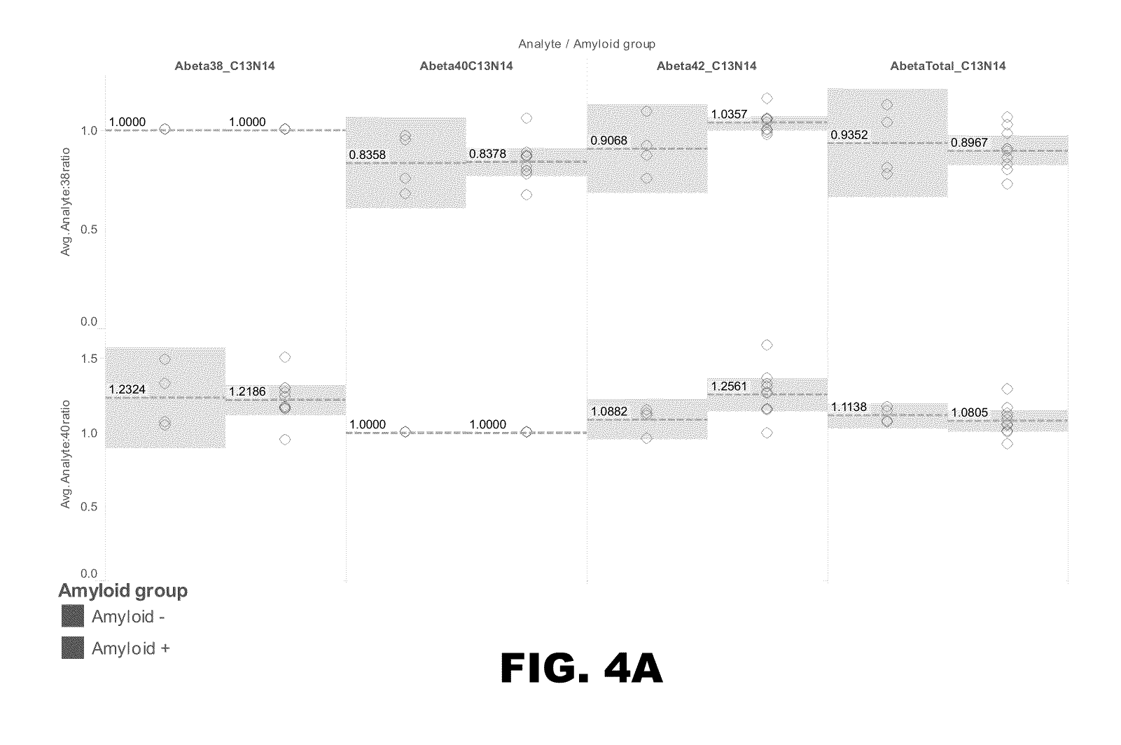

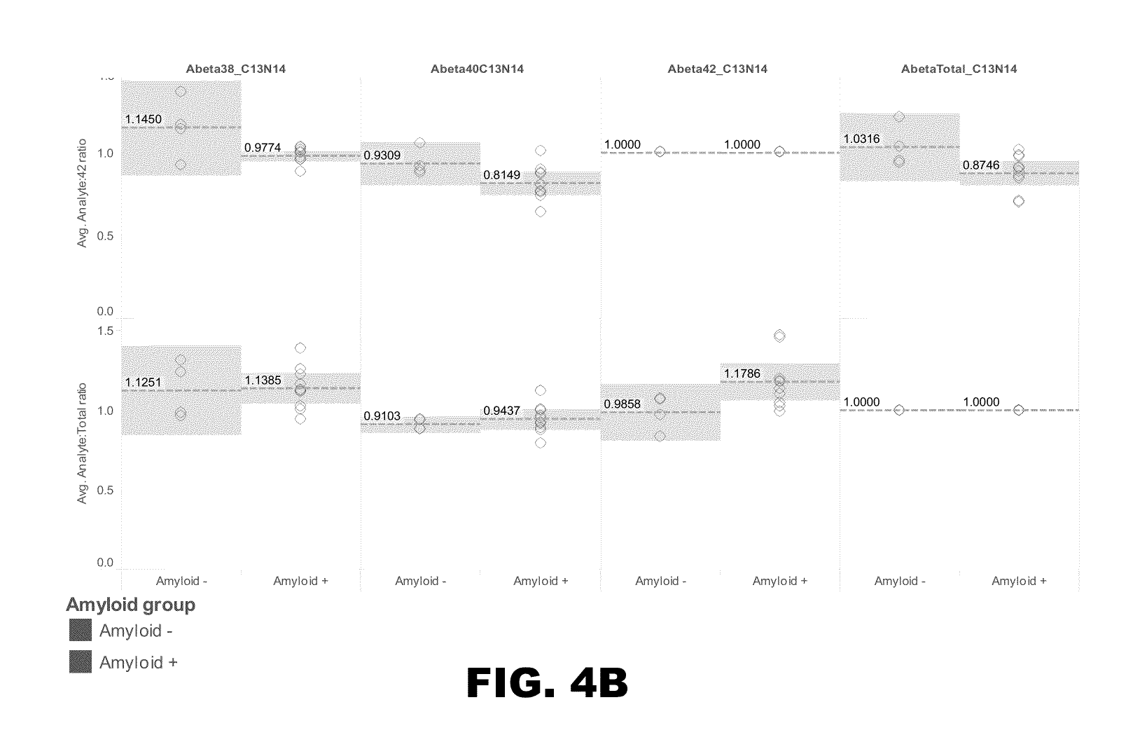

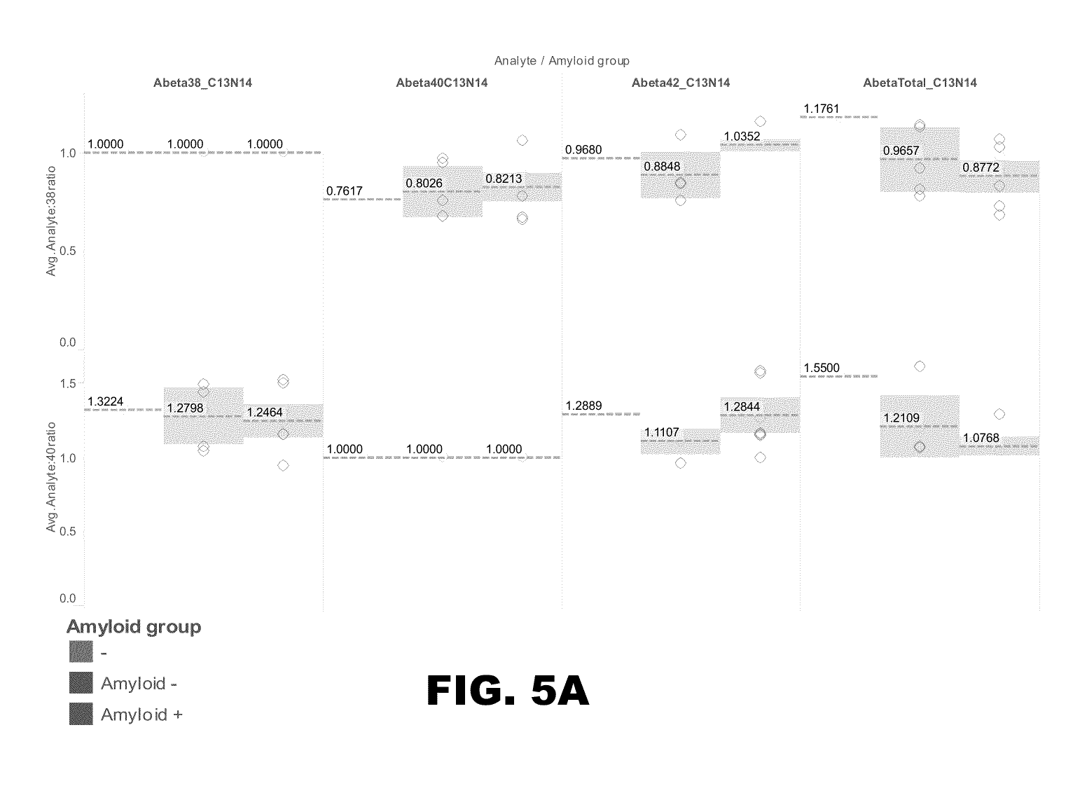

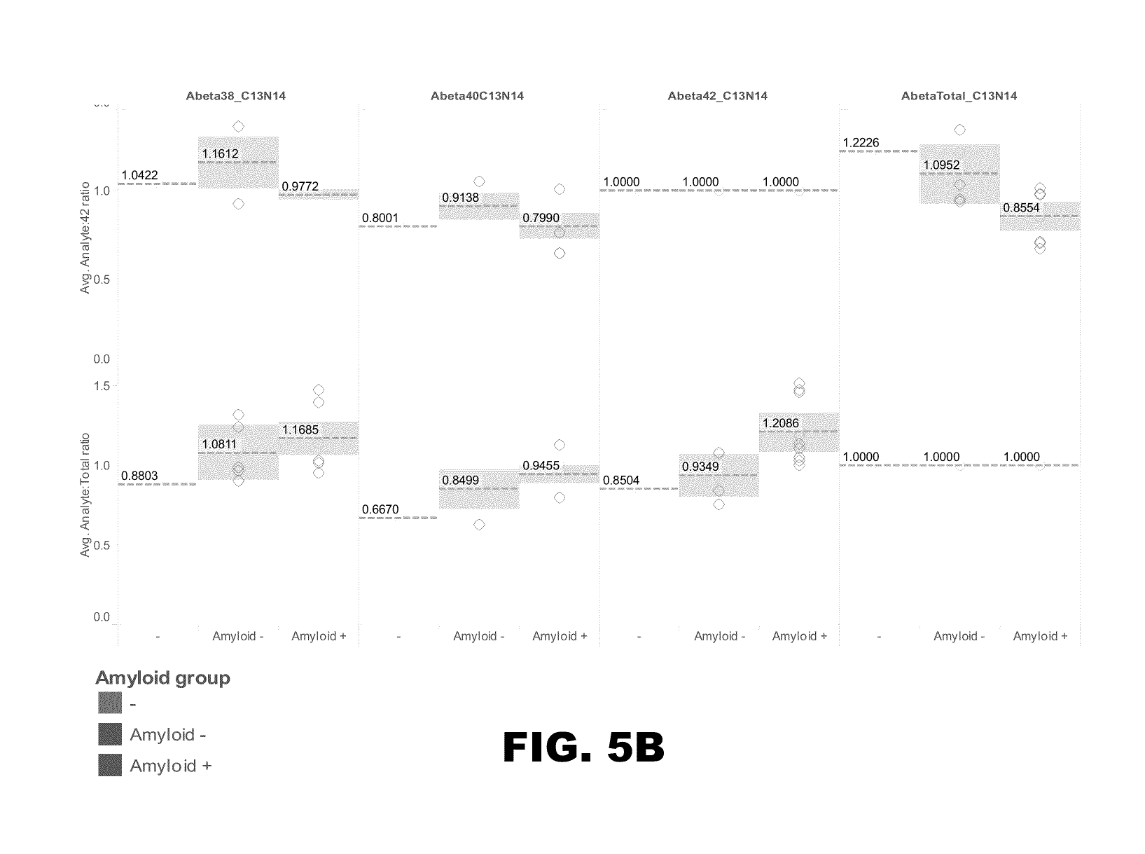

Interestingly, the data show for the first time that Aβ42/40 ratios in plasma varied significantly different by amyloid (PIB/CSF Aβ42) status at hours˜1-3 and ˜16-24 in a very similar fashion to CSF changes (Ab42 leads Ab40 during labeling and then falls faster than Ab40 during clearance/washout). As show in FIGS. 2 and 4, hours 1.5, 2, and 2.5 show significant Aβ42/40 increases between amyloid positive and amyloid negative patients. Hours 13-24 show significant decreases in Aβ42/40 between amyloid positive and amyloid negative patients (FIG. 5). No significant differences were observed between labeling protocols.

### Example 2

### IV Bolus and Oral Bolus are Equally Effective Labeling Protocols

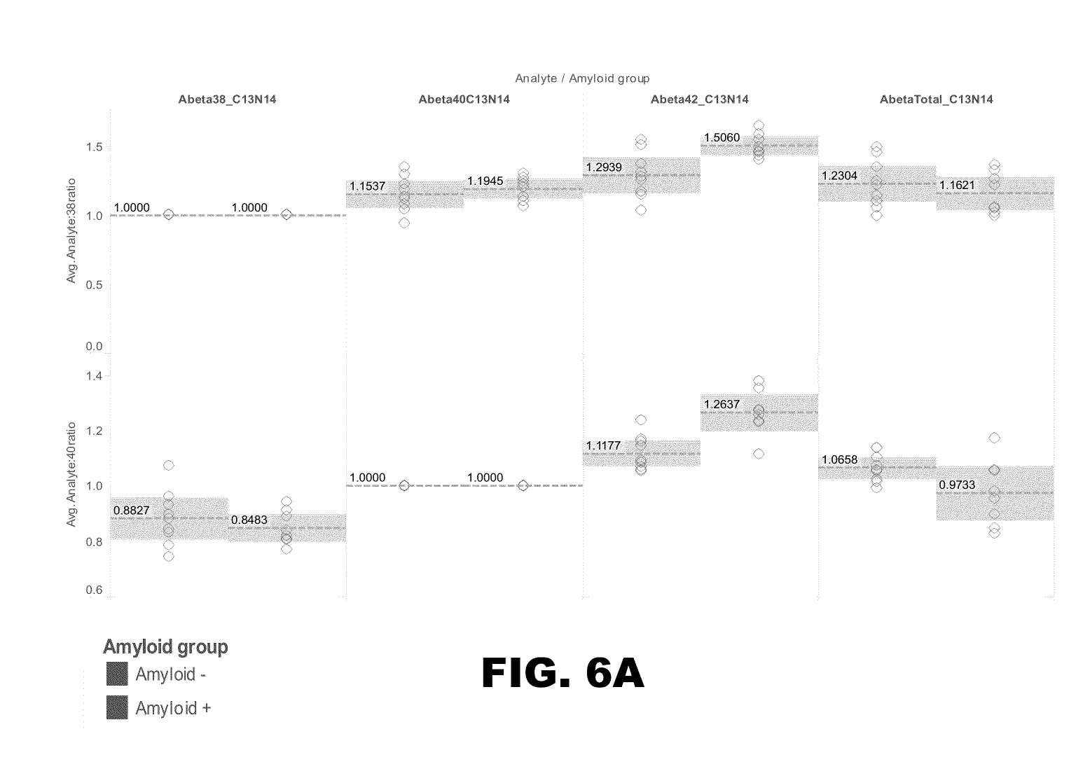

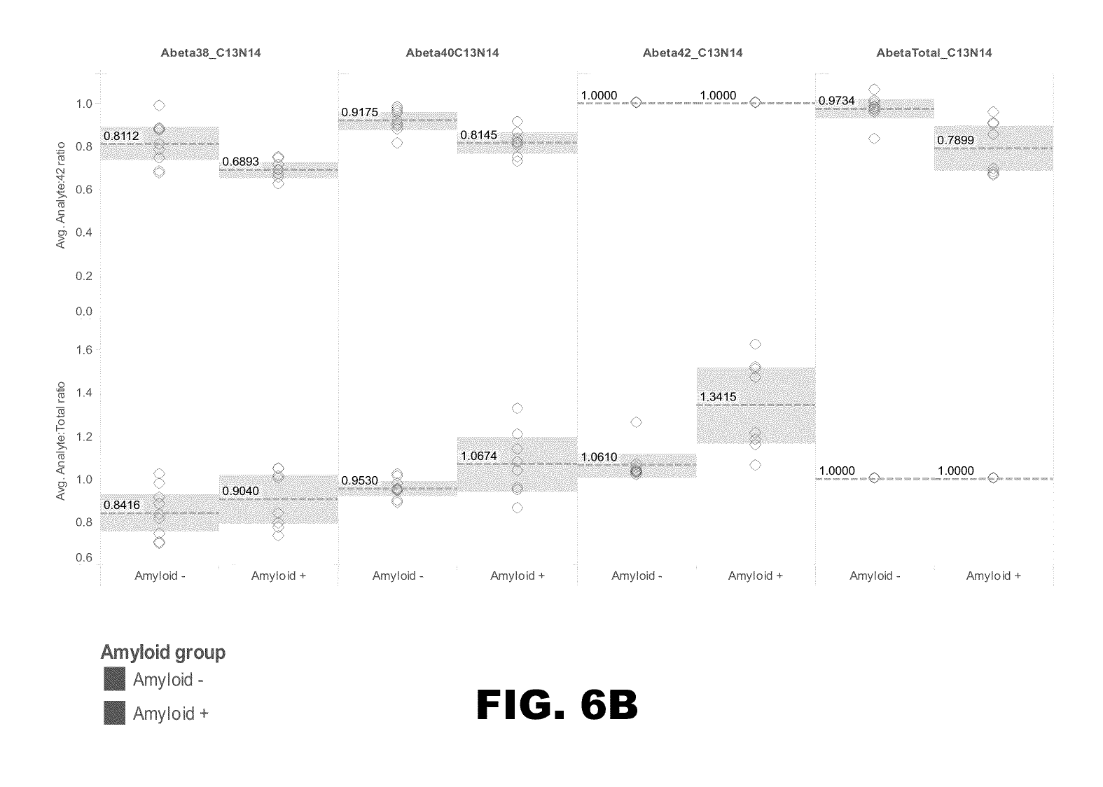

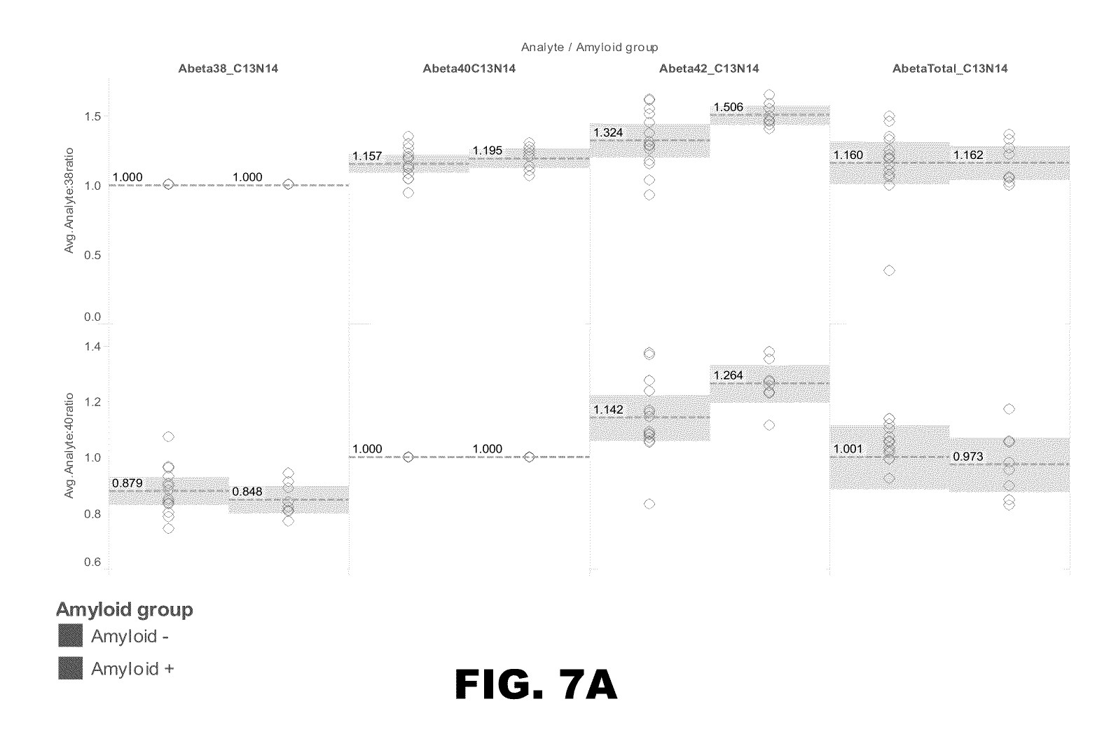

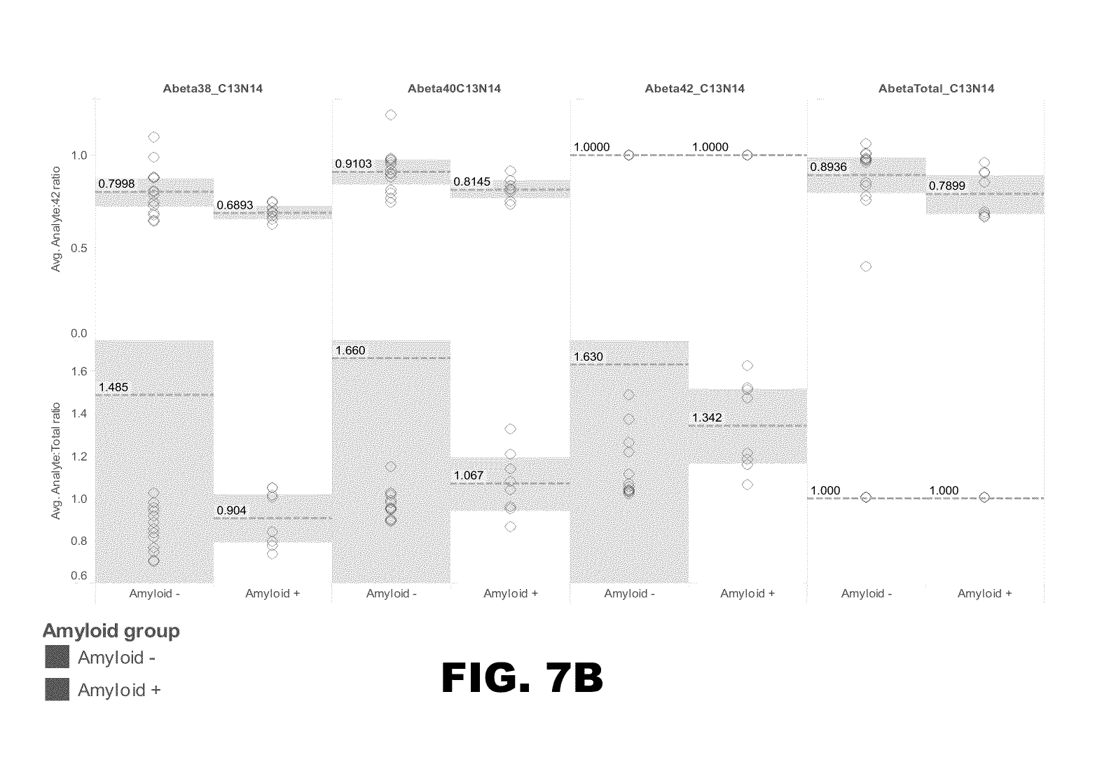

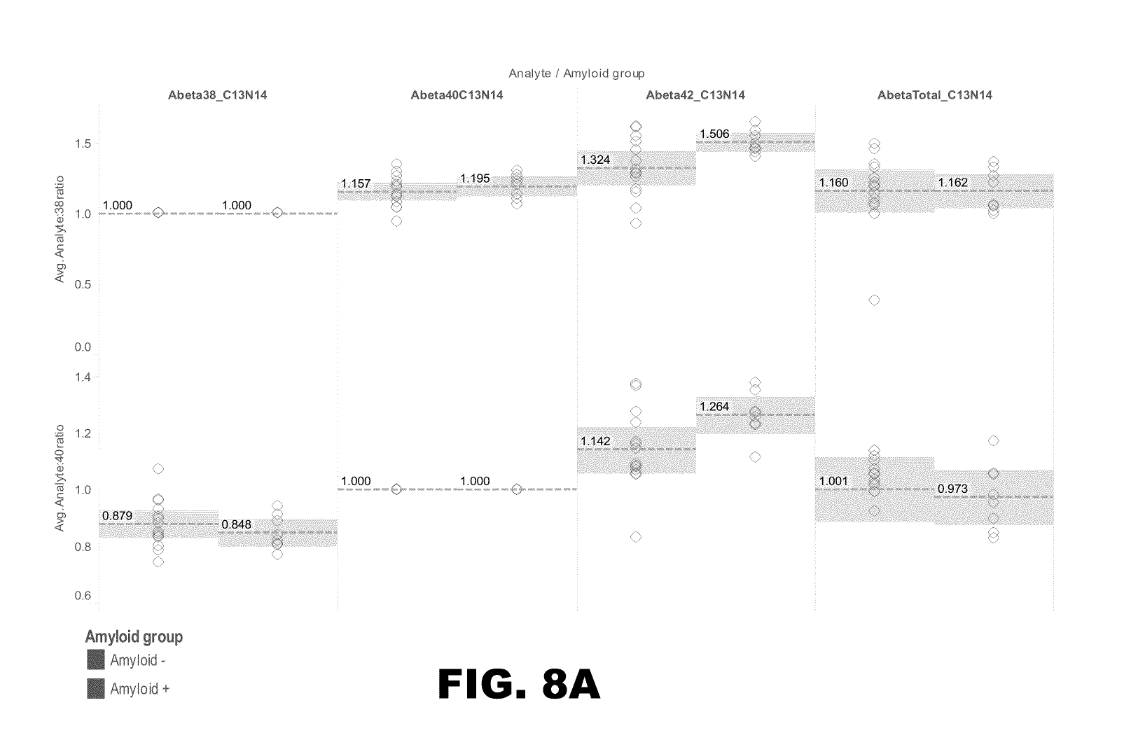

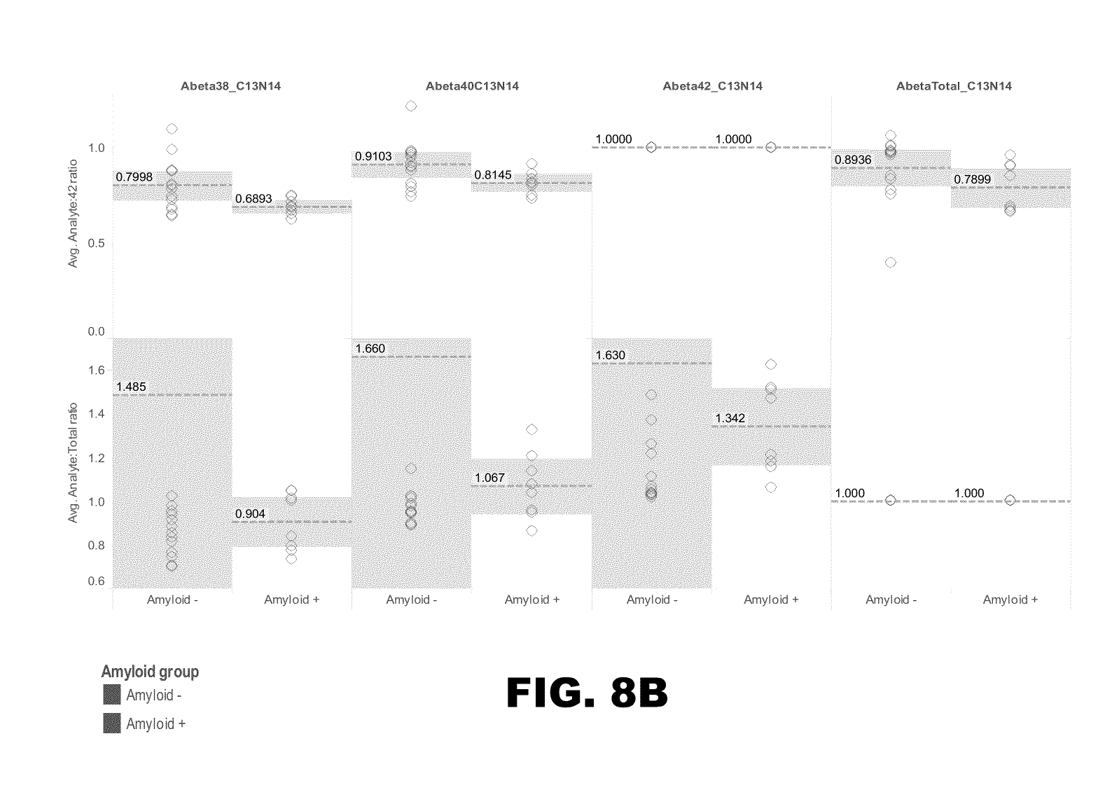

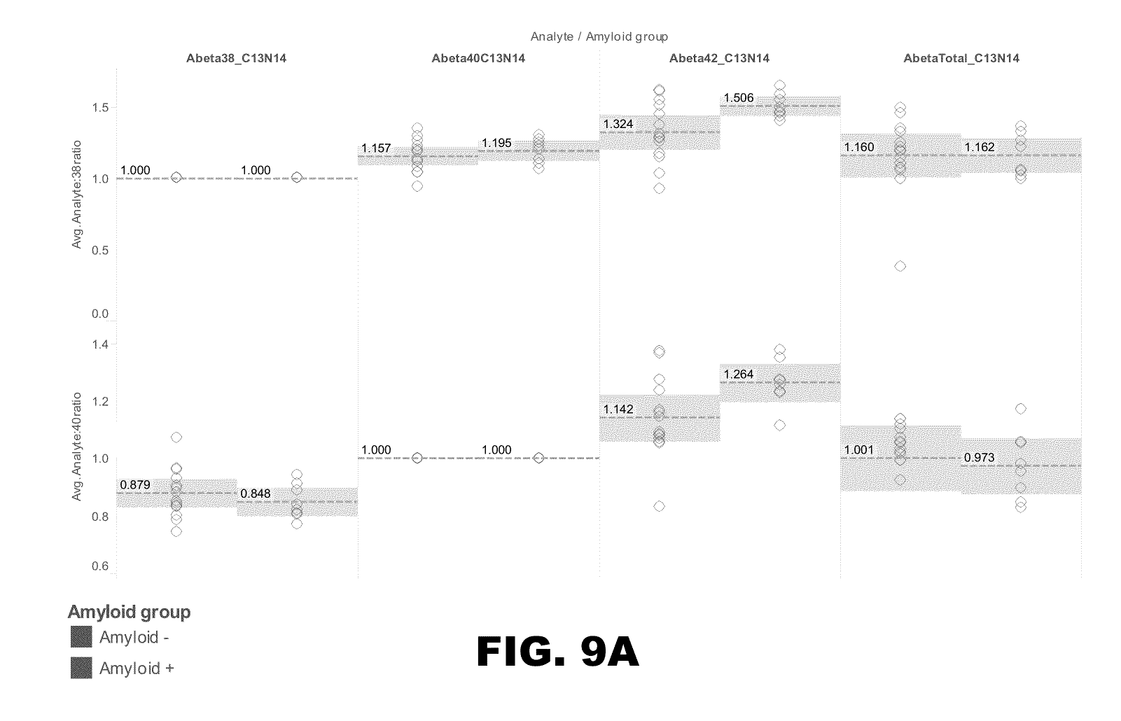

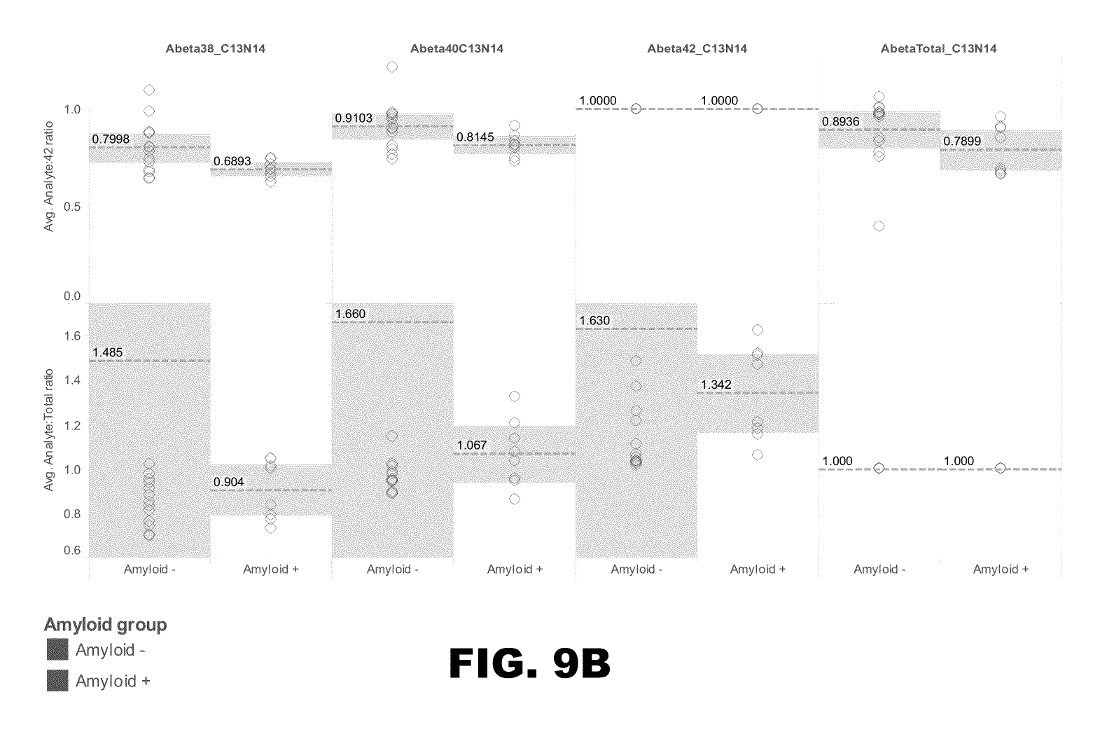

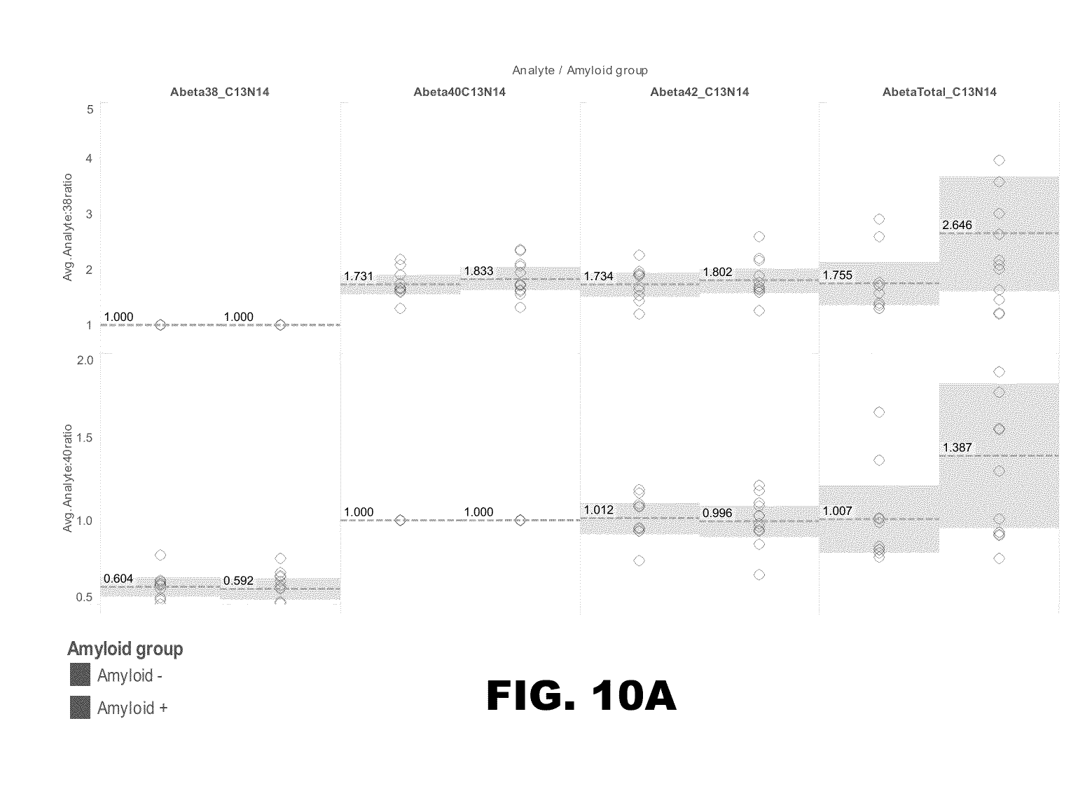

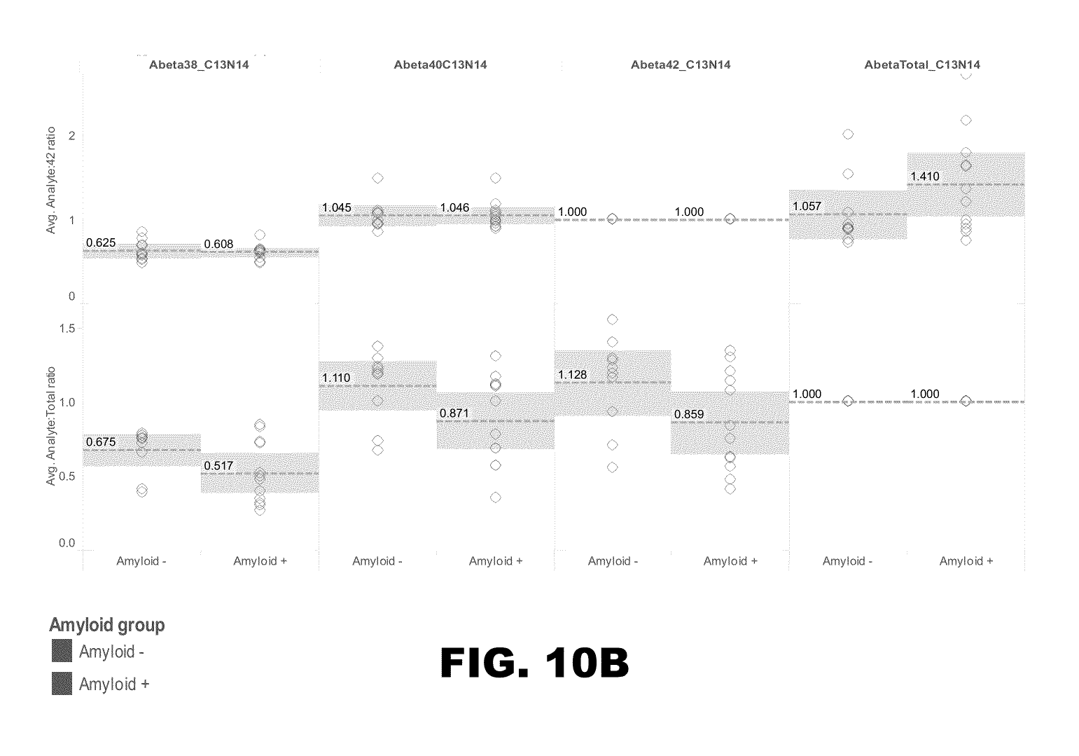

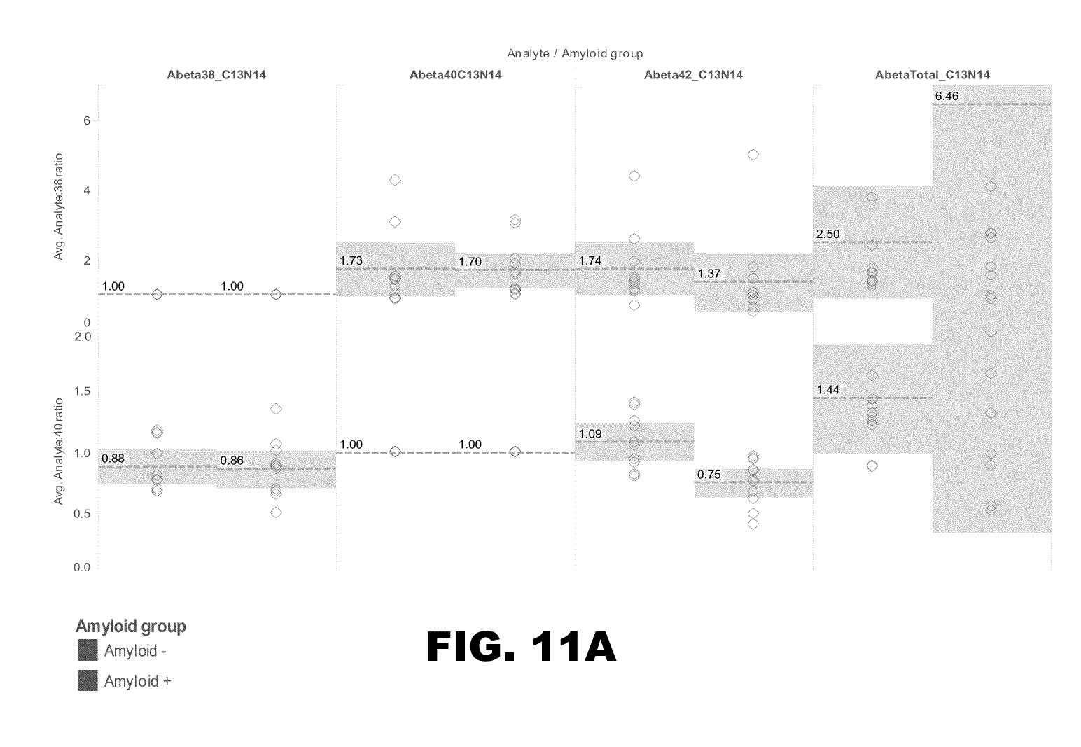

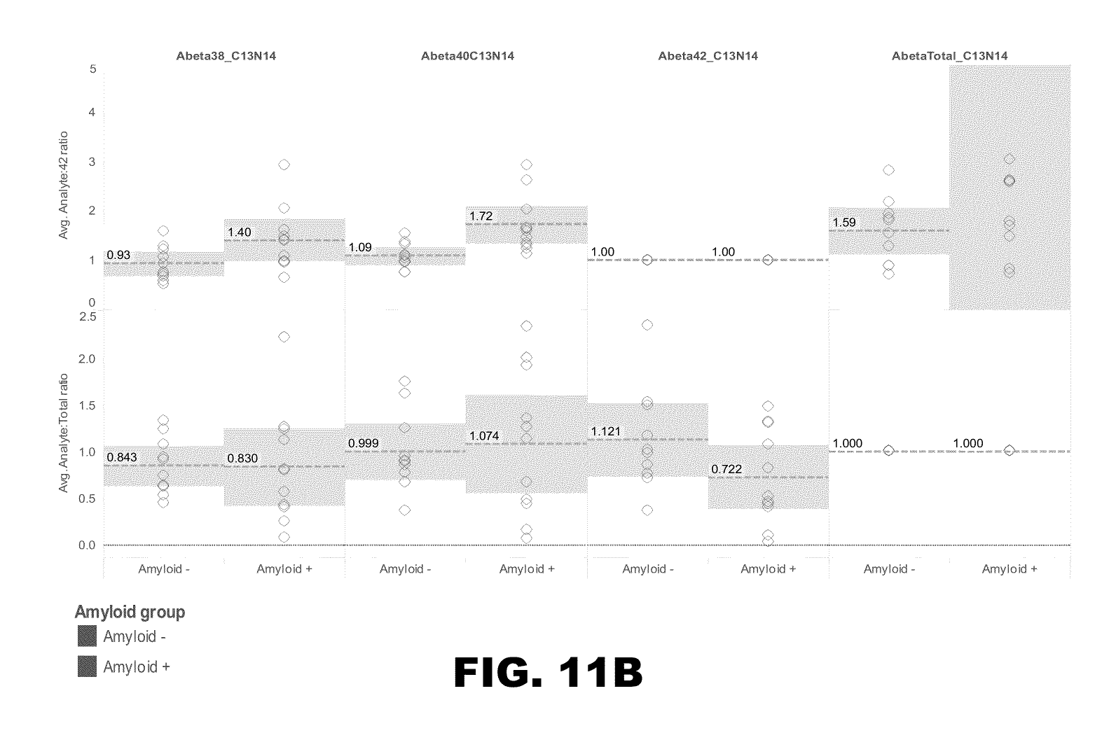

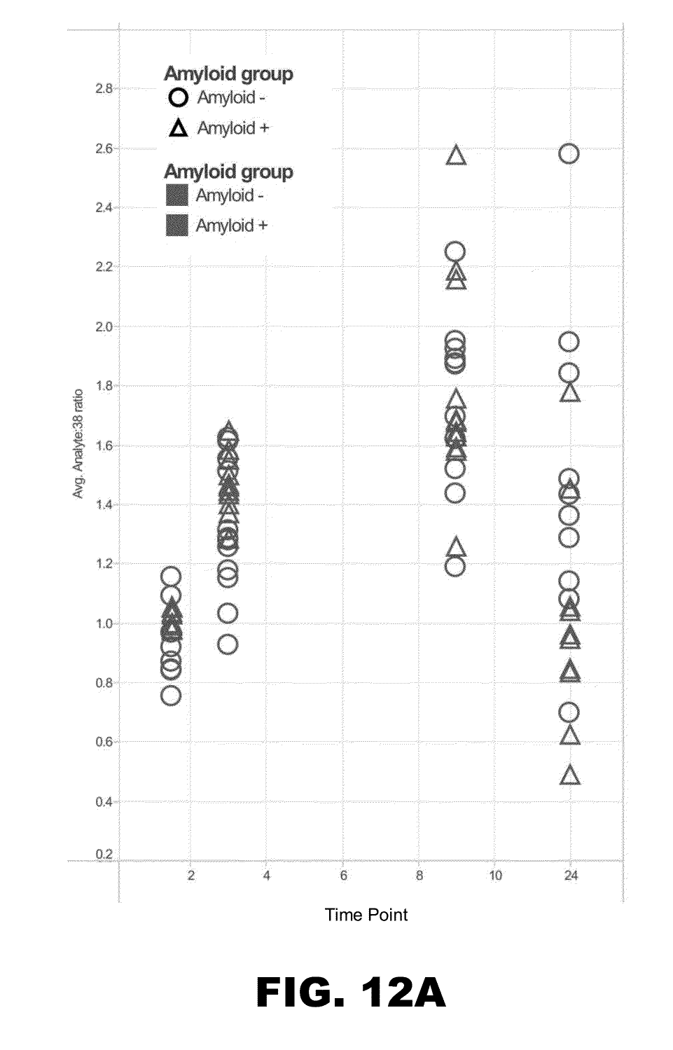

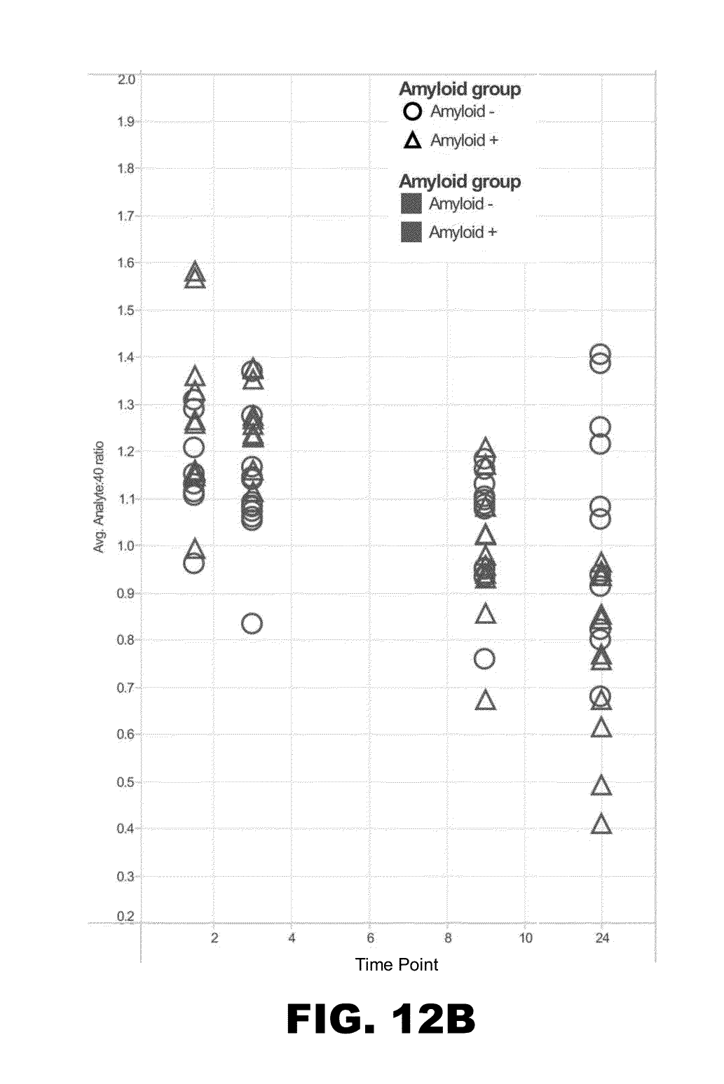

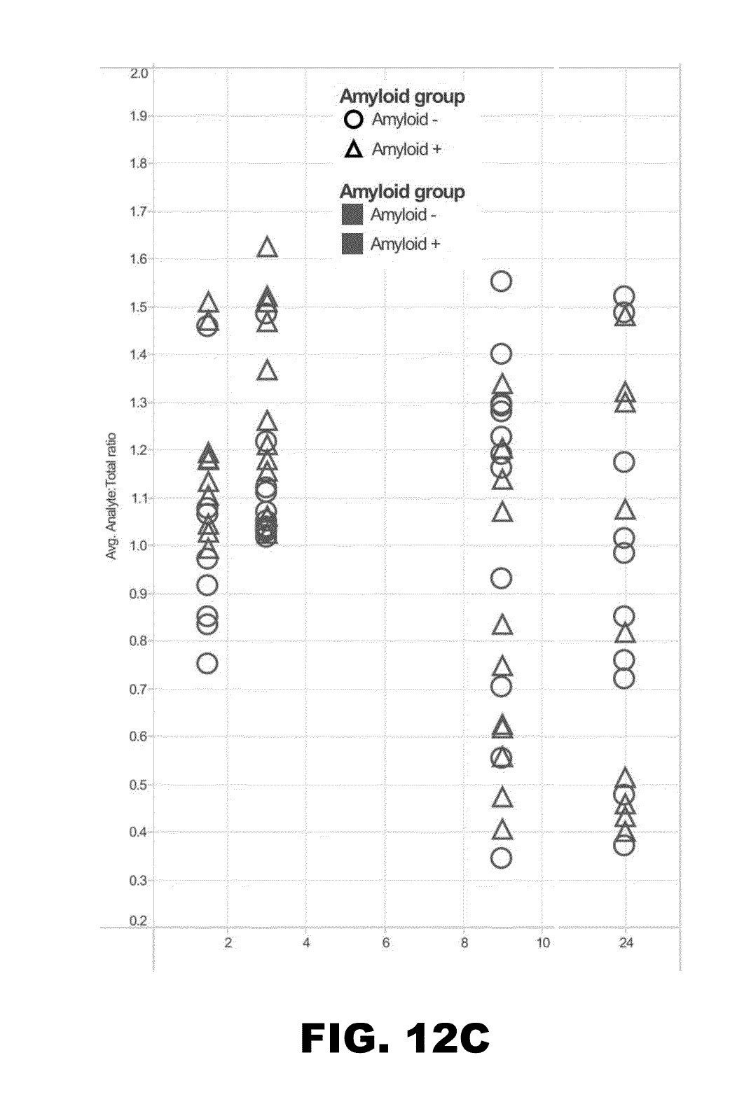

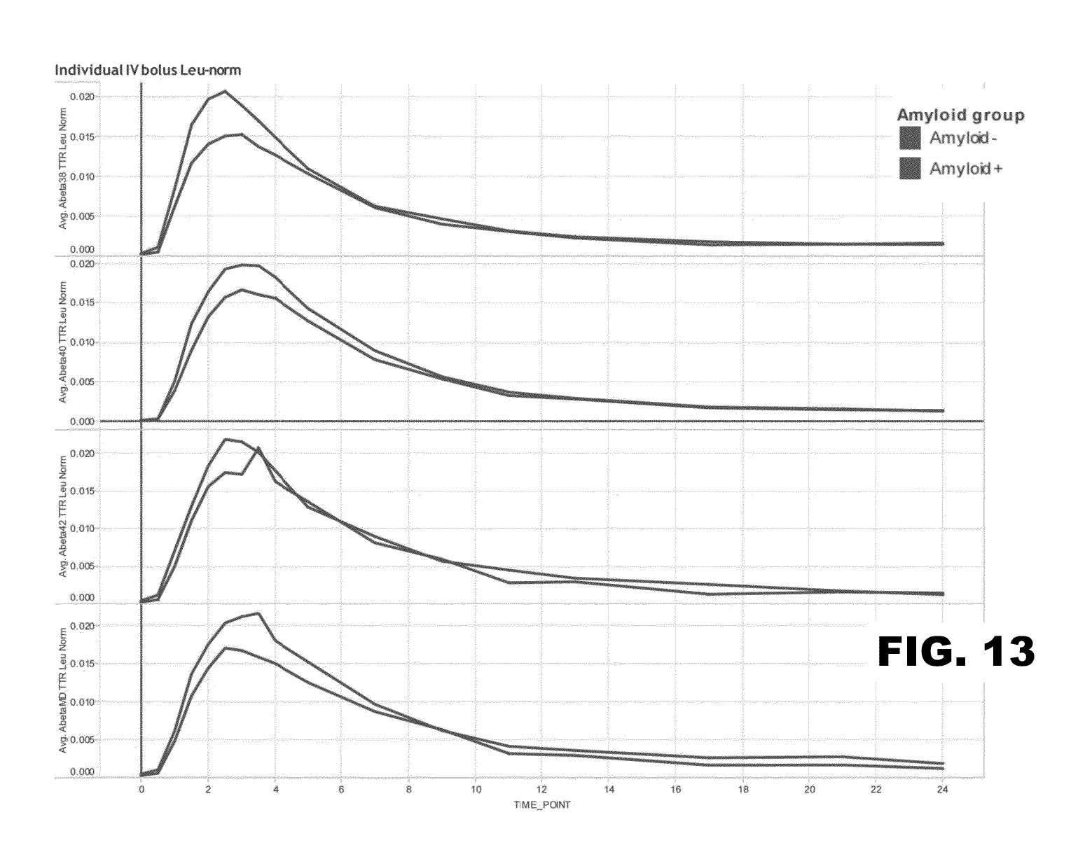

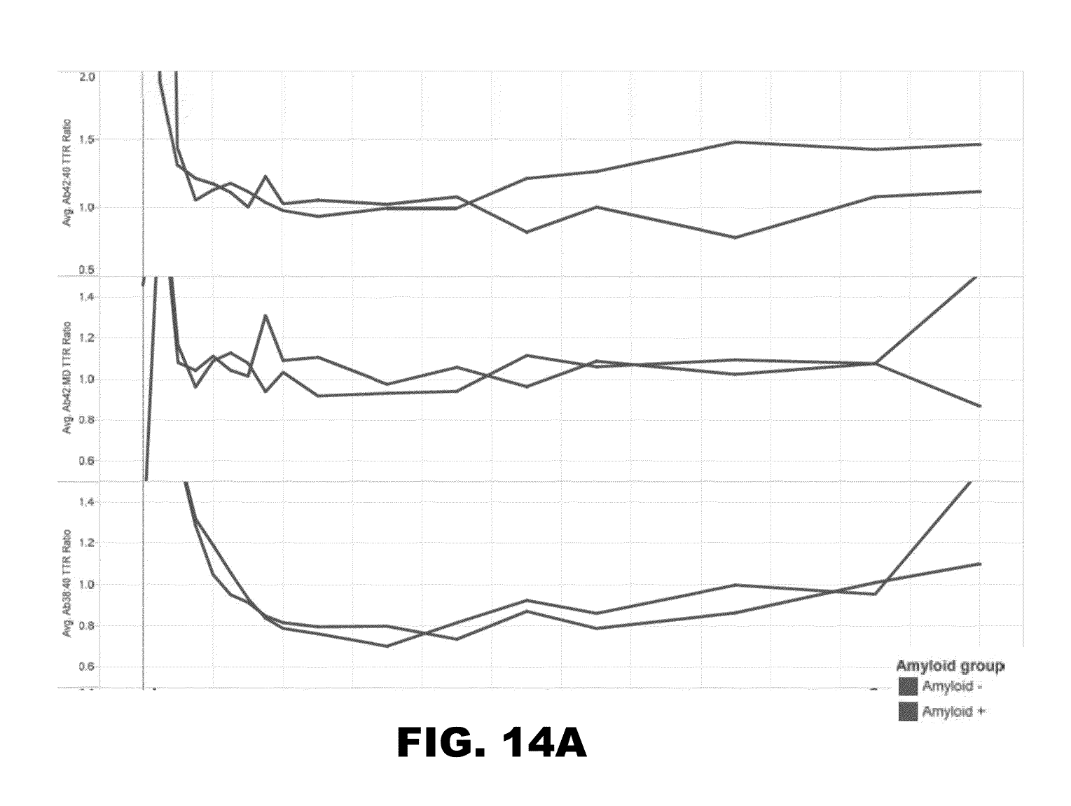

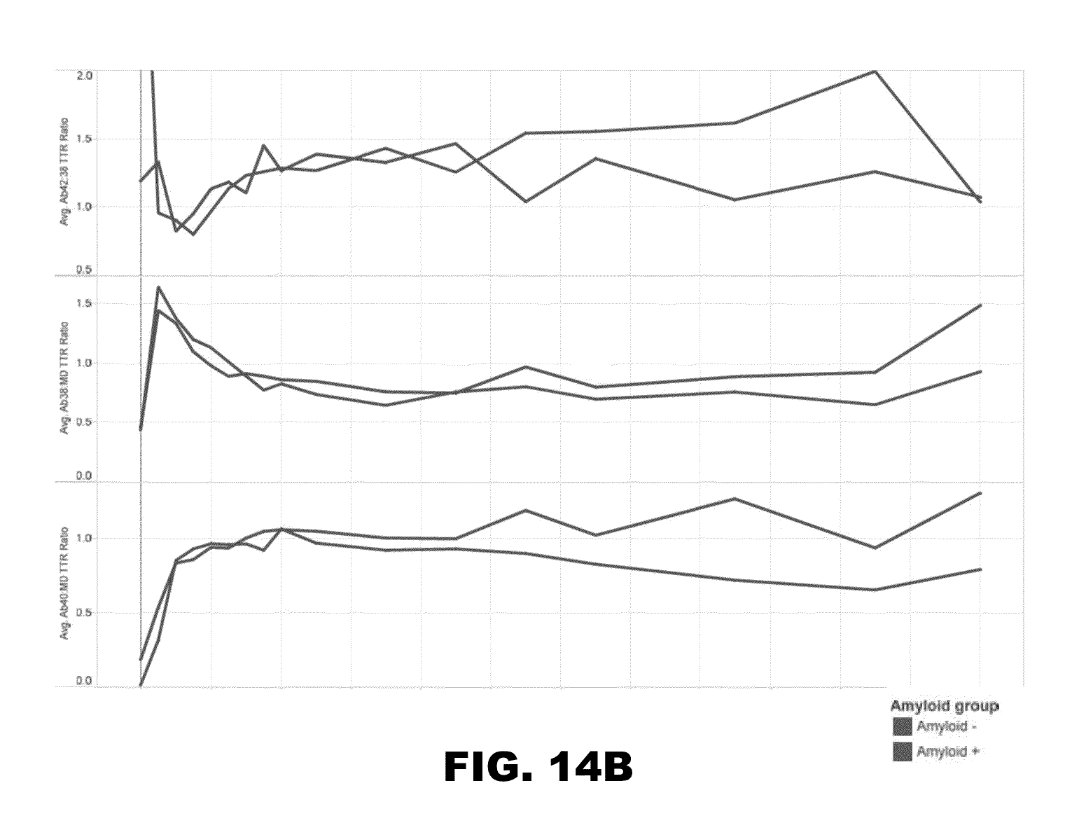

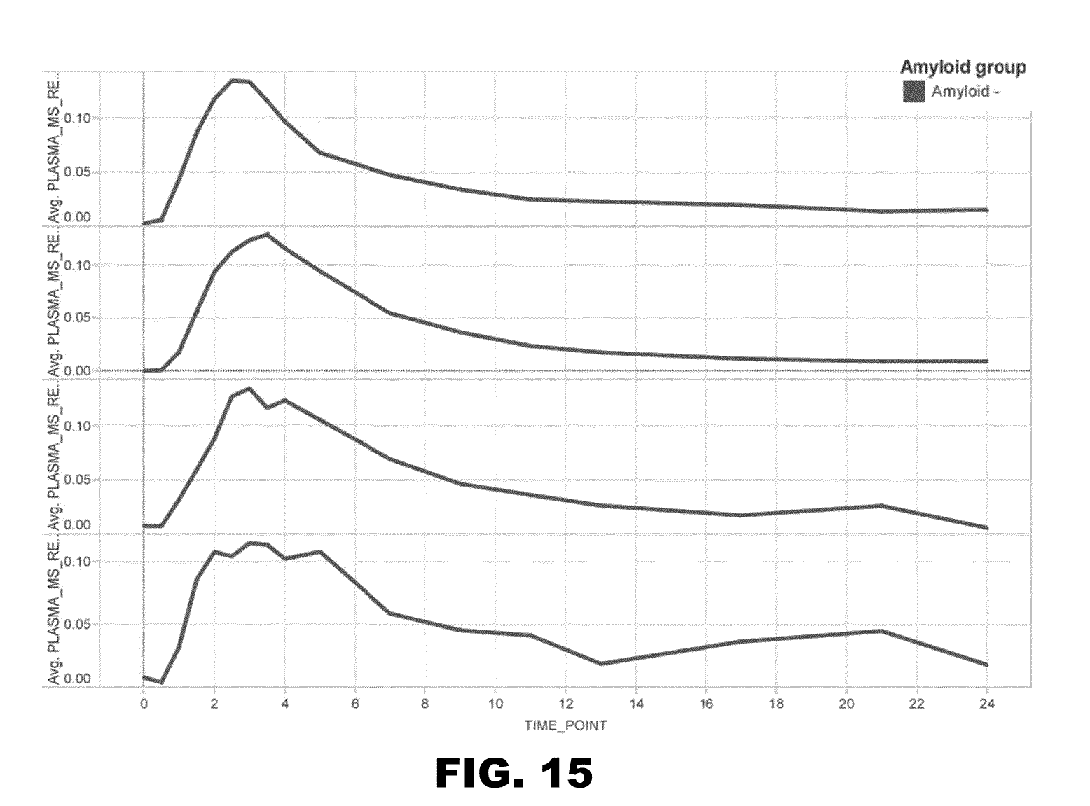

Late onset AD bolus: 31 total subjects (28 with known amyloid status by PET PIB scan or CSF amyloid-beta 42 concentrations) were given either an IV bolus or oral bolus of 800 mg of ⁶C₁₃ leucine at time zero. The IV or oral bolus was applied over less than 10 minutes. At multiple time points blood samples were drawn, centrifuged and frozen. Plasma samples were then immunoprecipitated for amyloid-beta which was processed according to protocol. Both labeled and unlabeled Aβ isoforms were quantified and shown for selected hours at 1.5 hr, 3 hr, 9 hr and 24 hr of labeling. FIG. 4-15: Note the significant differences in the Aβ42:Aβ40 TTR ratios, Aβ42: Aβtotal TTR ratios, Aβ42: Aβ38 TTR ratios and in Aβ38:Aβ40 or Aβ: Aβtotal ratios by each hour. Specific findings include increased Aβ42:Aβ40, Aβ42: Aβ38, or Aβ42: Total Aβ TTR ratios during early hours (1.5, 3) during increasing abeta labeling with decreased ratios at later hours (9 and 24) during decreasing abeta labeling. While Total Aβ to Aβ38, Aβ40 or Aβ42 was decreased in early hours and increased in later hours.

### Example 3

### CNS Aβ42 Kinetics are Altered with Amyloidosis

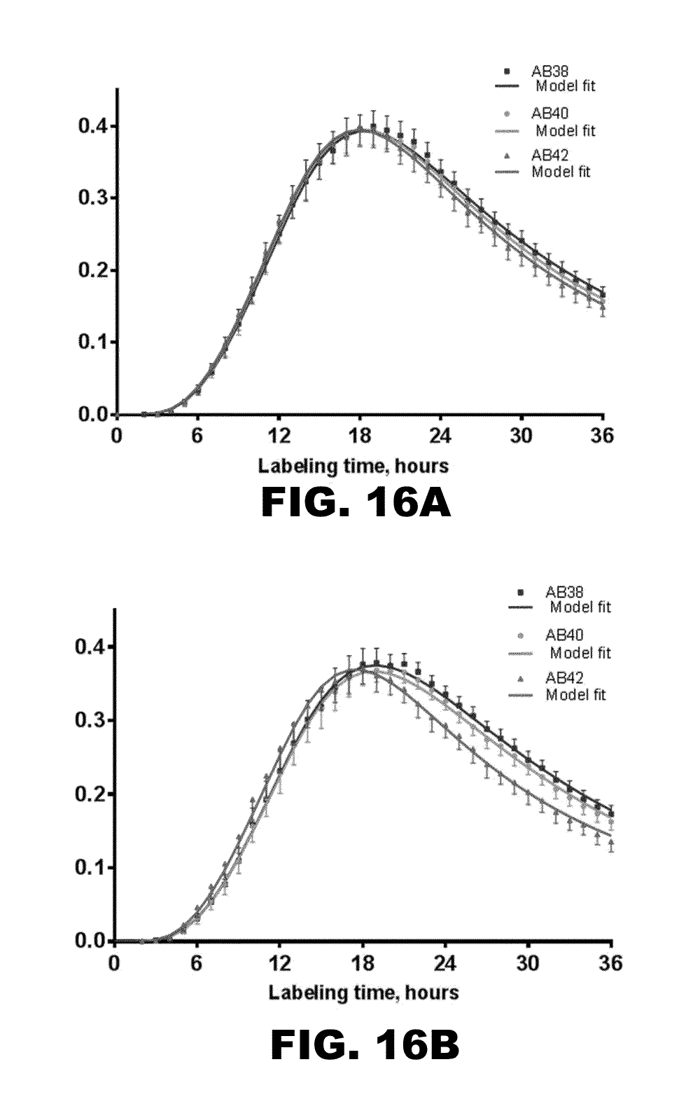

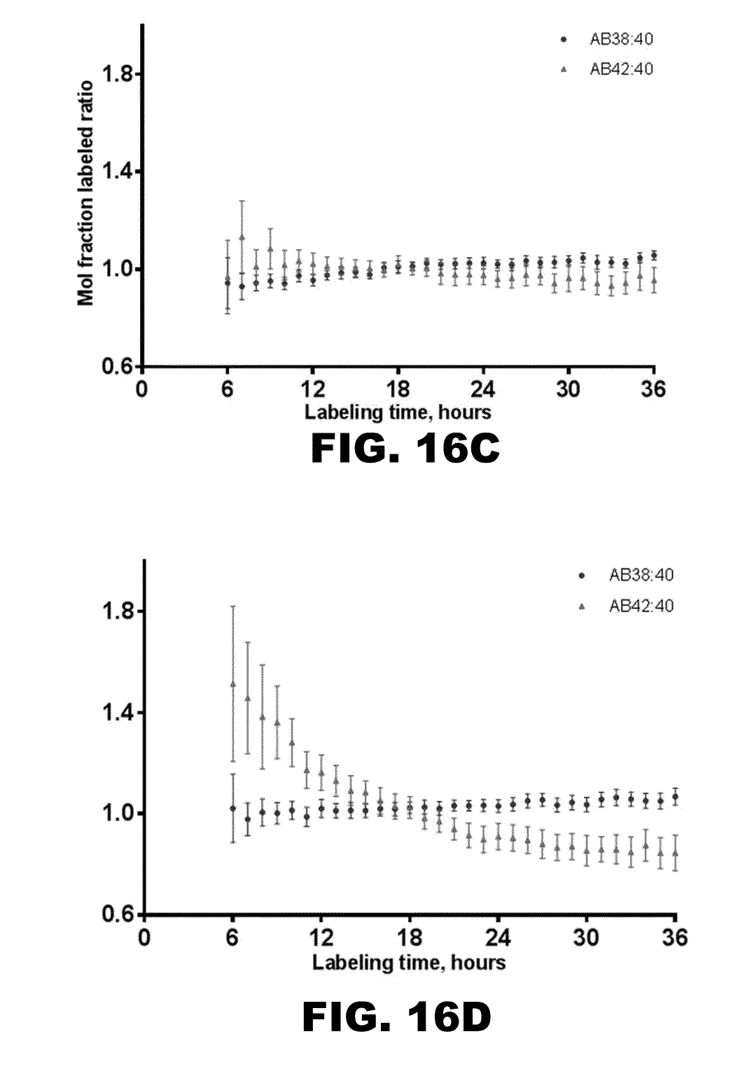

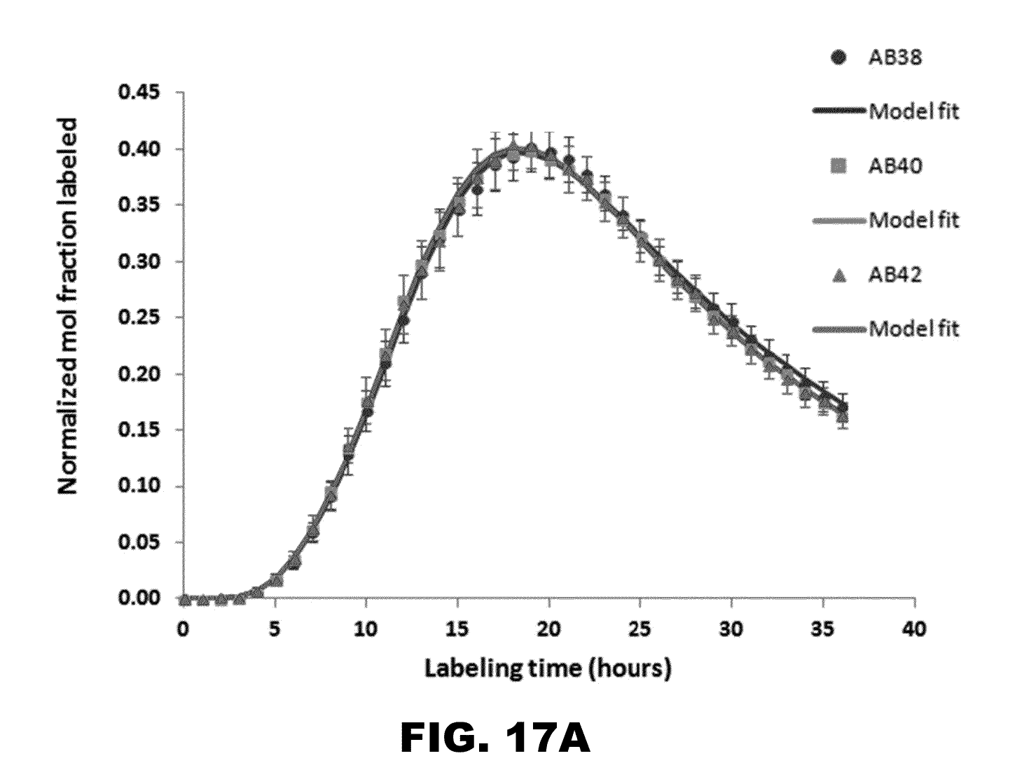

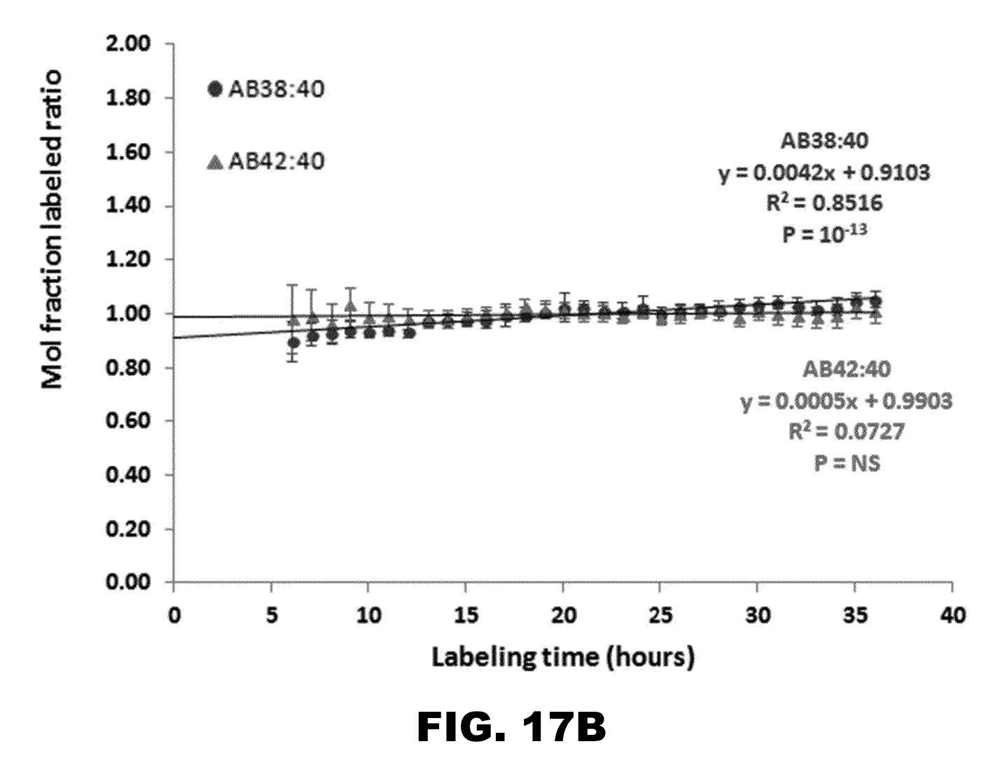

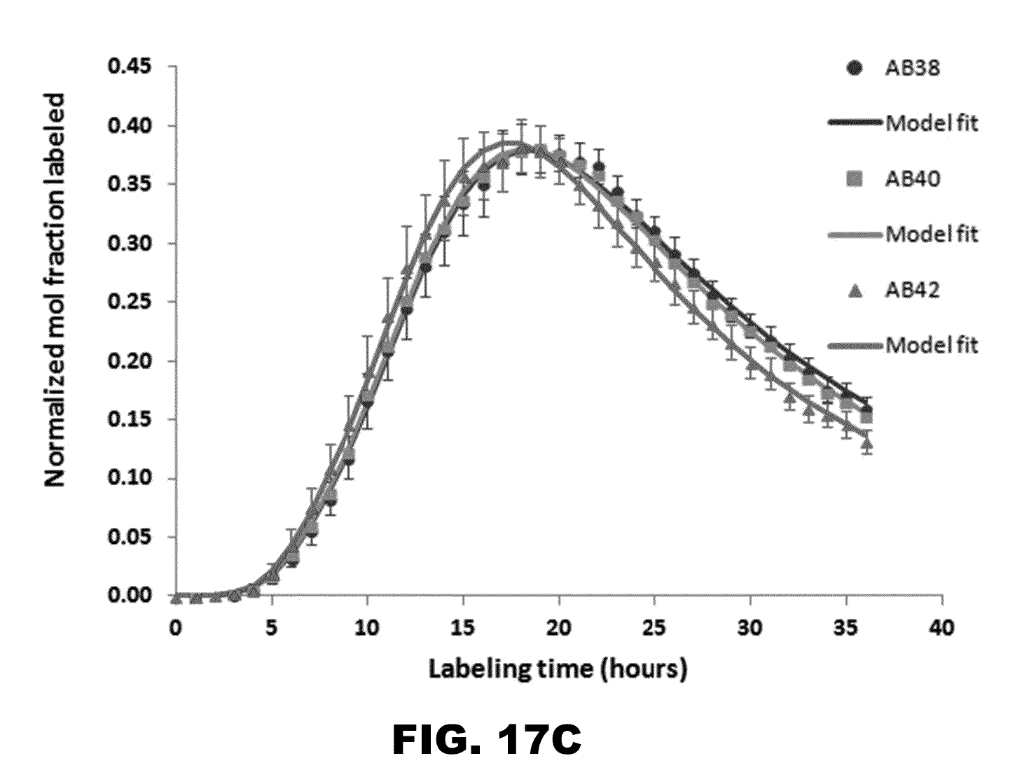

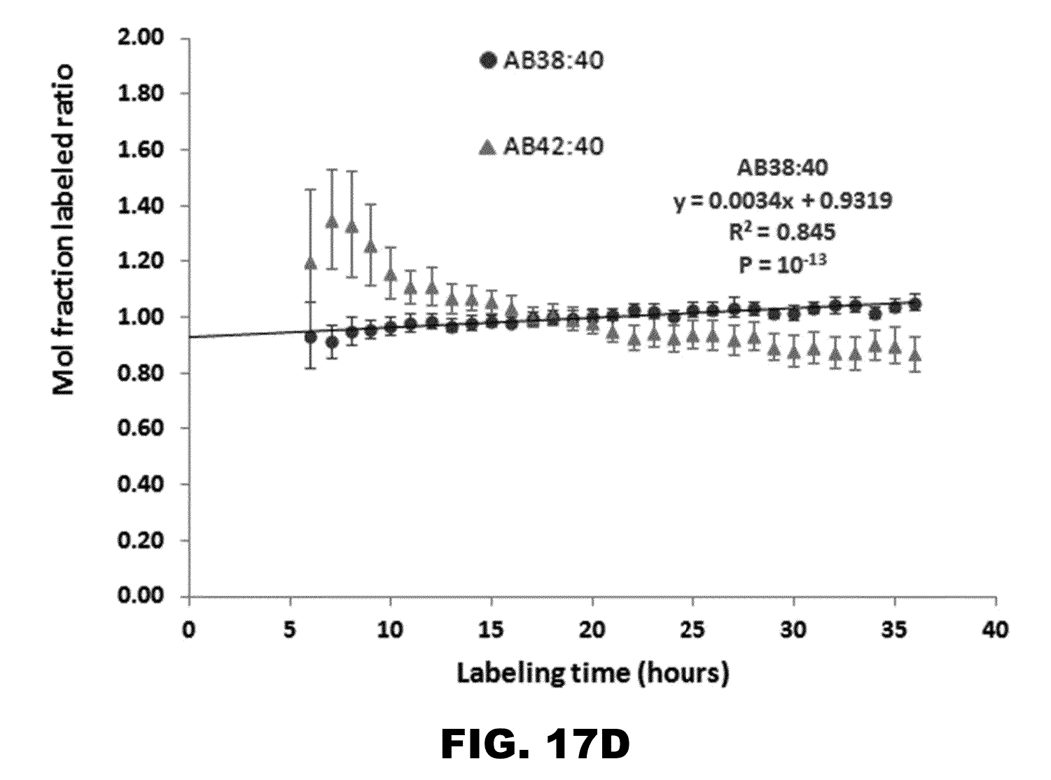

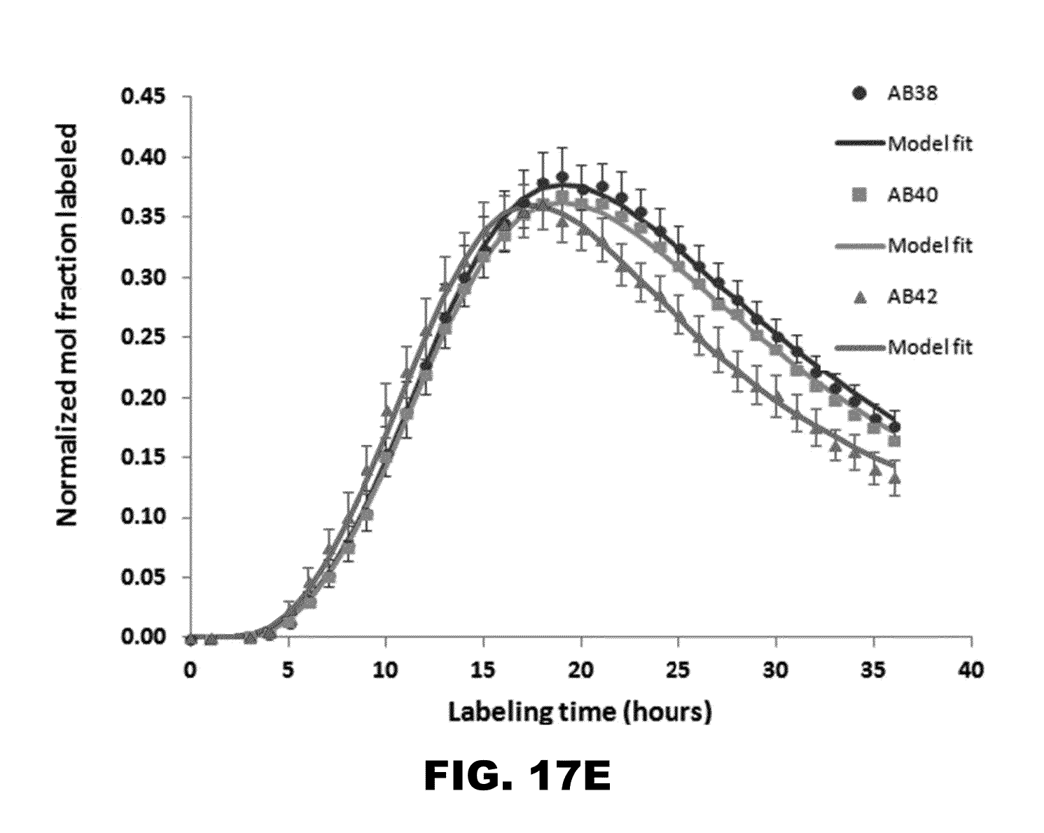

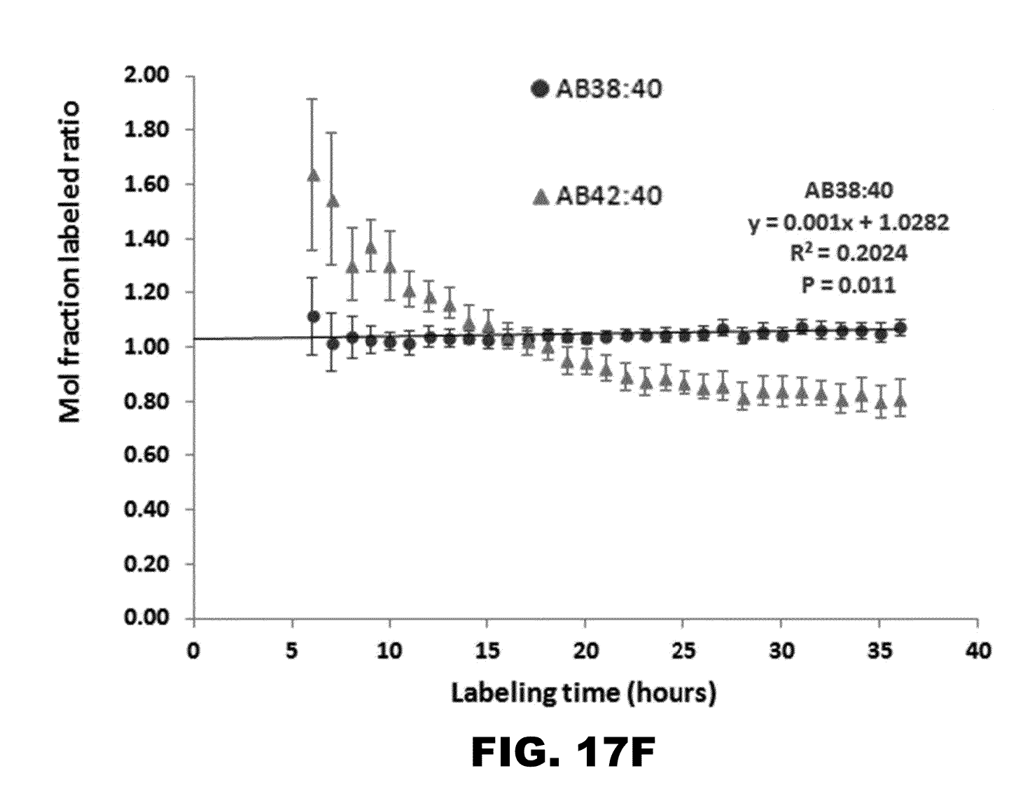

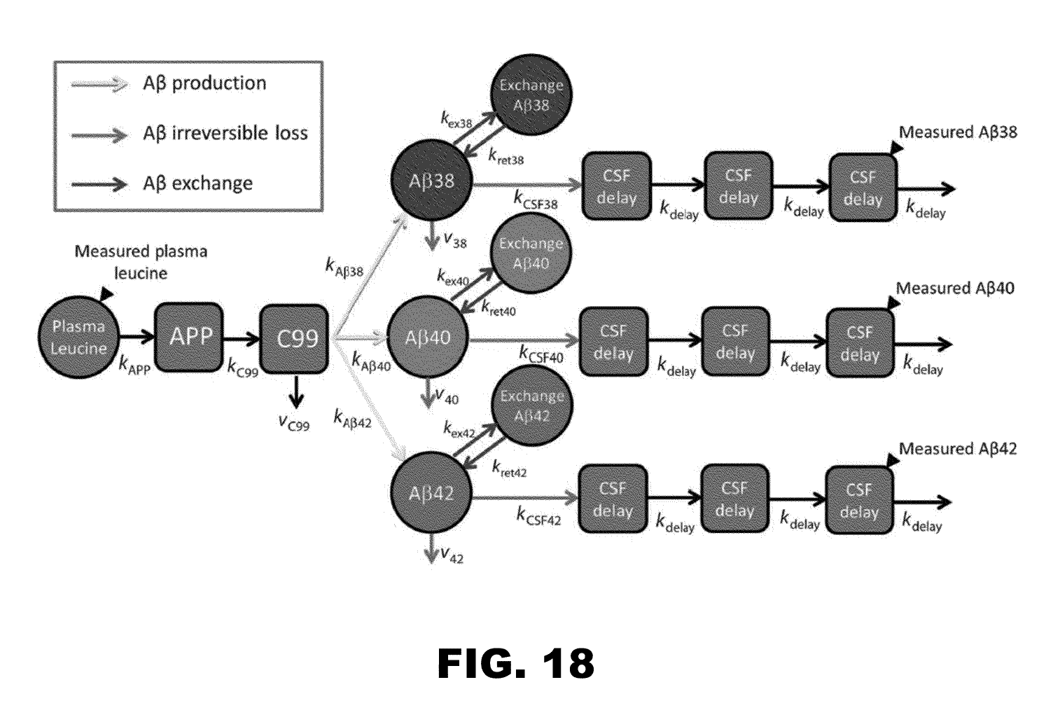

In order to understand the potential interaction between brain amyloidosis and soluble Aβ kinetics, Aβ38, Aβ40 and Aβ42 kinetics were quantified and compared between amyloid negative and positive participants matched for age (Table1). The SILK time course for Aβ38, Aβ40 and Aβ42 were similar in amyloid negative participants. The SILK Aβ38 and Aβ40 time courses were similar in amyloid positive participants; however, Aβ42 labeling kinetics peaked significantly earlier than Aβ38 and Aβ40 in amyloid positive participants (FIG. 16, Table 1). To compare Aβ SILK time courses between amyloid positive and negative participants, Aβ isotopic enrichment ratios were calculated (FIG. 16B). This revealed the Aβ38/Aβ40 isotope enrichment ratio was close to 1.0 throughout the time course in both amyloid negative and positive groups, indicating the same kinetics of Aβ38 and Aβ40 with regard to amyloid status. However in the amyloid positive group, the SILK Aβ42/Aβ40 ratio was greater than one during the rise to peak labeling and less than one after the peak (FIG. 16B), consistent with a prior report of faster soluble Aβ42 labeling kinetics associated with amyloidosis in ADAD.¹⁴ This indicated a specific disturbance in soluble Aβ42 kinetics in the amyloid positive group only. Aβ42 SILK alterations were evident in some amyloid negative participants; so we examined the Aβ SILK profiles by tertiles of CSF Aβ42/Aβ40 concentration ratios which revealed similar findings. The anomalous Aβ42 SILK peak morphology for Aβ42 was completely absent in the 34 participants with CSF Aβ42/Aβ40 concentration ratios>0.16, was evident with concentration ratios between 0.10-0.16, and most pronounced in participants with ratios<0.1 (FIG. 17).

A compartmental model was used to determine the kinetic parameters of Aβ38, Aβ40, and Aβ42 turnover kinetics (see Methods¹⁴ and FIG. 18). The model has three main parameters that are adjusted to fit the kinetic data: the fractional turnover rate (FTR), reversible exchange (kₑₓ), and delay (k_{delay}). Turnover of the soluble Aβ peptides is characterized by the FTR, representing the irreversible loss of soluble peptides (made up of transport from brain to CSF fluid, deposition into amyloid plaques, local tissue uptake, and proteolysis). Reversible exchange (kₑₓ) of isotopically labeled Aβ42 with previously existing (i.e. unlabeled) Aβ42 was needed to fit the bi-exponential labeling decay curve in participants with amyloidosis. The delay rate constant (k_{delay}) accounts for the approximate nine hour delay between cessation of isotope labeled amino acid infusion and the peak labeling of Aβ in CSF. The delay rate constant might reflect the rate of fluid flow through CNS and is taken to be equivalent for all Aβ isoforms. The model provided an excellent fit to the Aβ isoform labeling time courses in all participants (FIGS. 16, 17, and 19) with an average R2=0.991±0.007, 0.993±0.004 and 0.981±0.016 for Aβ38, Aβ40, and Aβ42, respectively.

The FTR of Aβ42 was significantly faster in amyloid positive compared to amyloid negative participants (0.112±0.035 vs. 0.094±0.031 pools/h, P=0.011; Table 1). The kinetic anomaly of Aβ42 is more pronounced in amyloid positive participants when inter-subject variability is reduced by normalization to the FTR of Aβ40, as the Aβ42/Aβ40 FTR ratio was ˜1.4 (P=10-10 for amyloidosis status; Table 1).

An exchange process of Aβ42 was evident in amyloid positive participants (exchange rate constant 0.049±0.054 pools/h, approximately half the magnitude of Aβ42 FTR), but nearly absent in amyloid negative participants (0.006±0.032 pools/h, P<10-5; Table 1). Thus, increased Aβ42 FTR and exchange appear to be the major kinetic alterations associated with amyloid plaques.

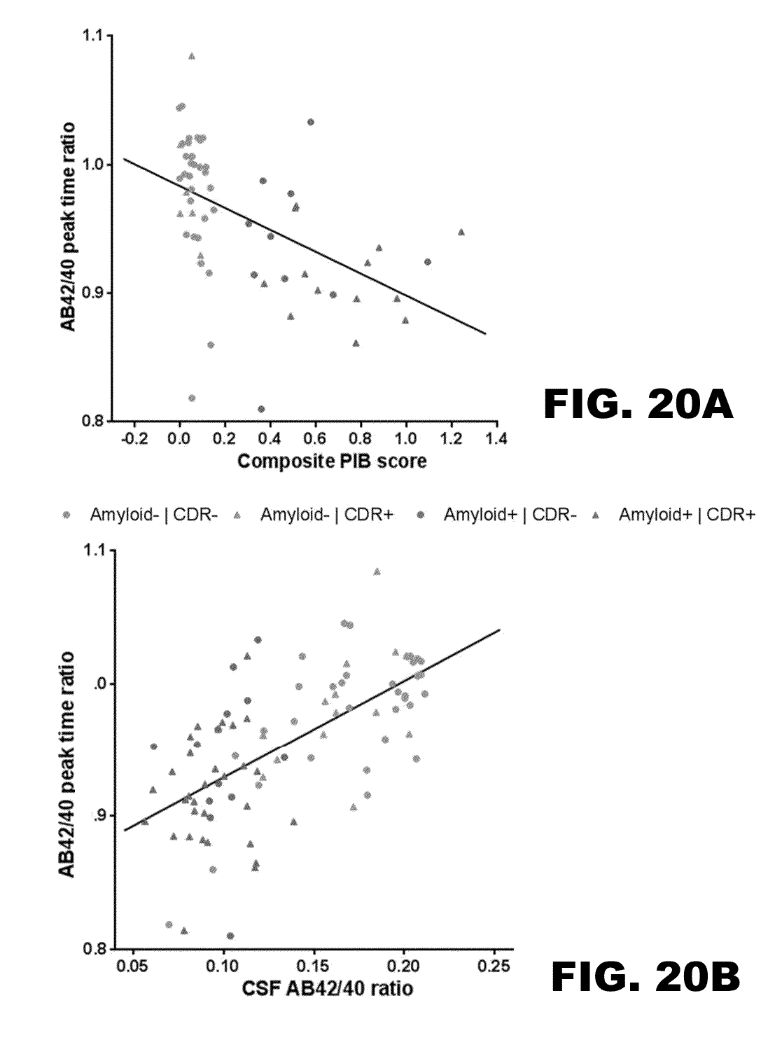

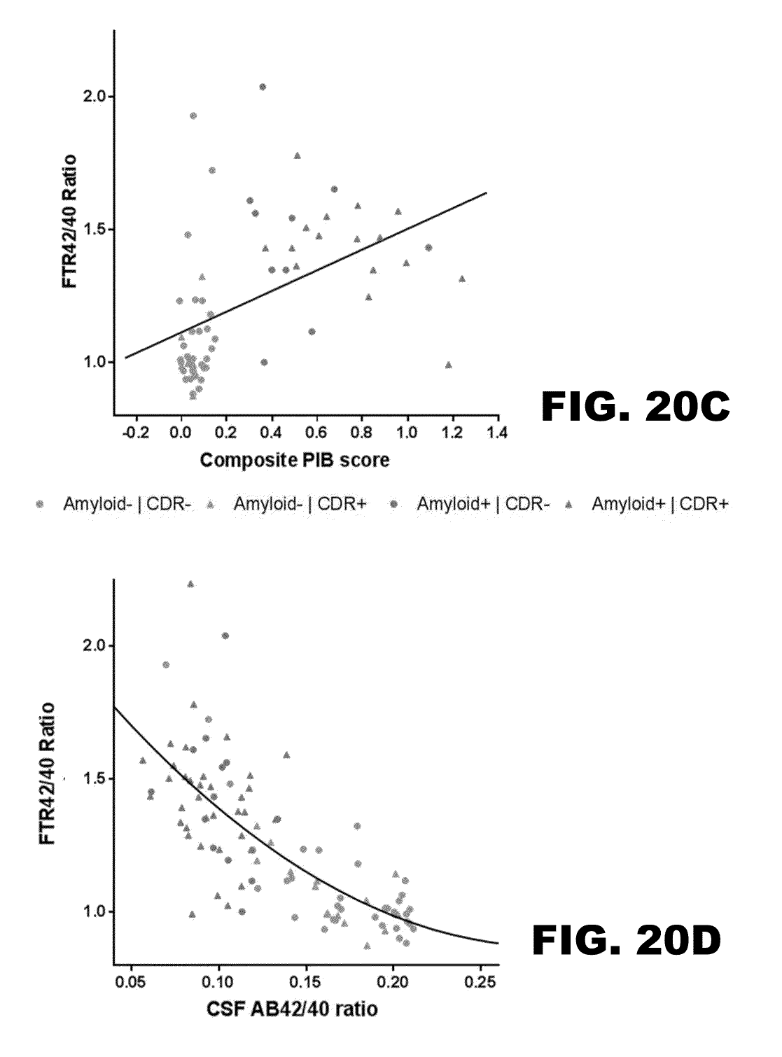

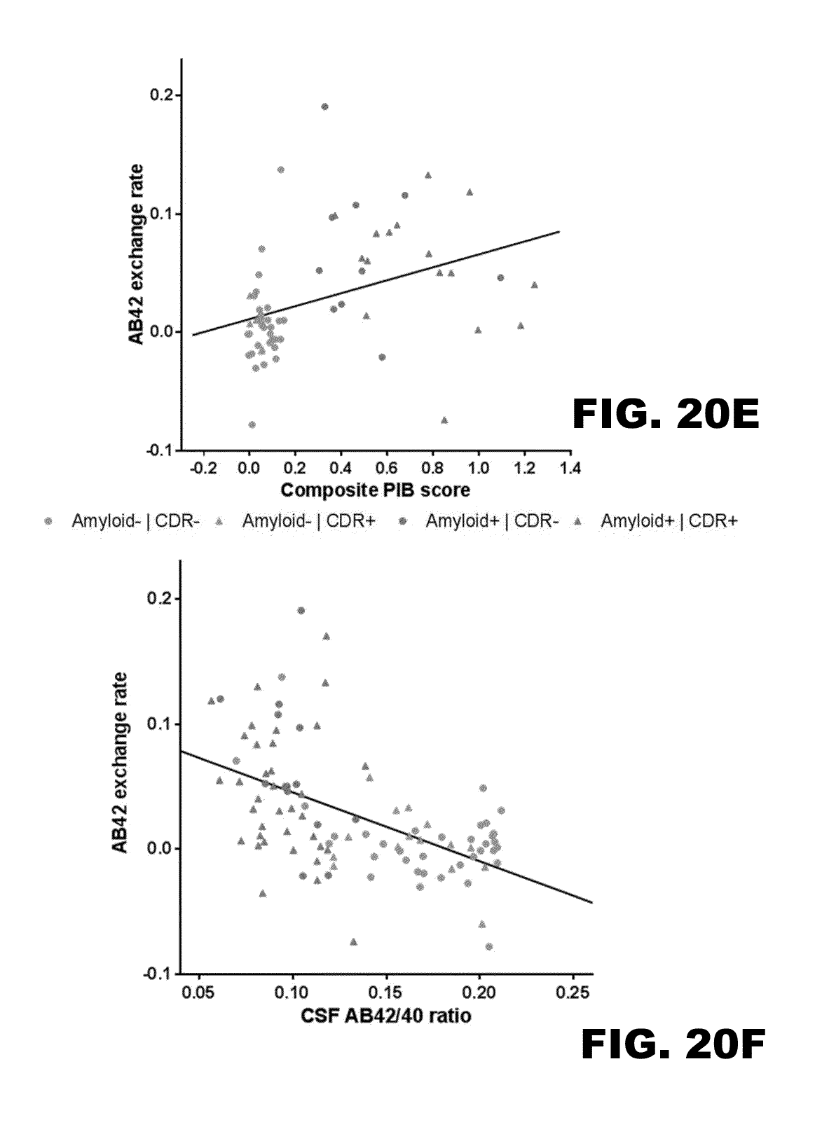

Correlation of Aβ SILK parameters and measures of amyloidosis identified linear correlations of Aβ42/40 peak labeling time ratios and PET PIB MCBP (r=−0.47) and CSF Aβ42/40 concentration ratios, (r=0.63, FIG. 20). FTR Aβ42/40 and Aβ42 exchange demonstrate a non-linear or state binary change relationship to amyloidosis (FIG. 20), suggesting these measures detect the absence or presence of amyloidosis, and the Aβ42/40 peak labeling time ratios more accurately quantify the amount of fibrillar amyloid plaques.

Table 1A. Associations between subject characteristics and Aβ kinetics with amyloidosis and cognitive impairment.

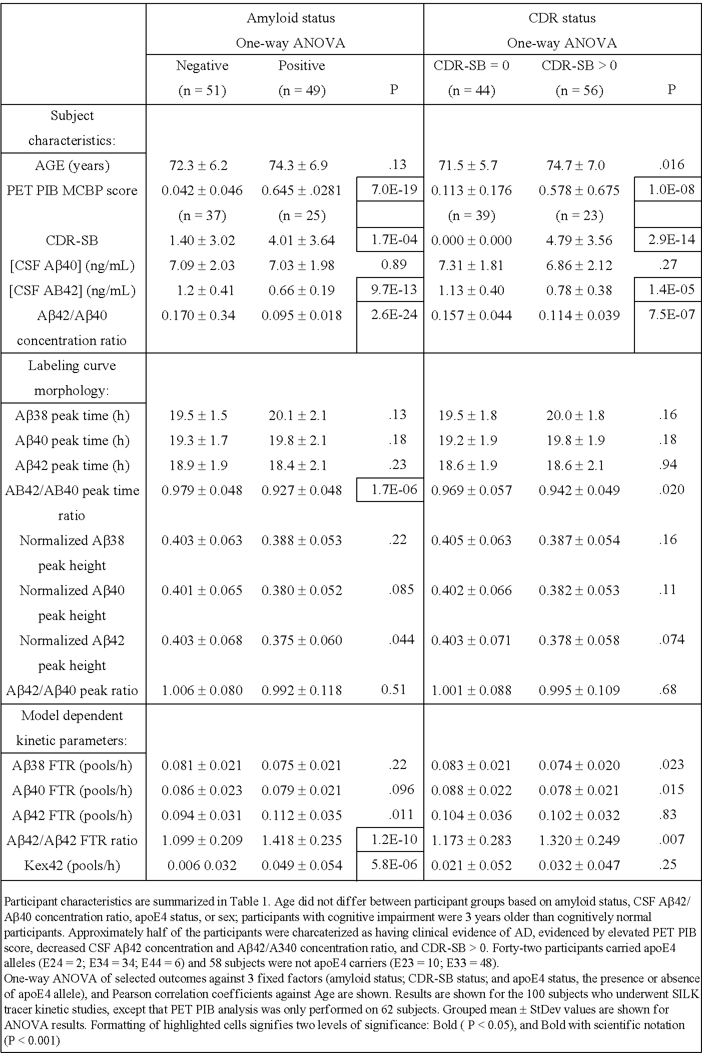

Table 1B. Associations between subject characteristics and Aβ kinetics with apoE4 and age.

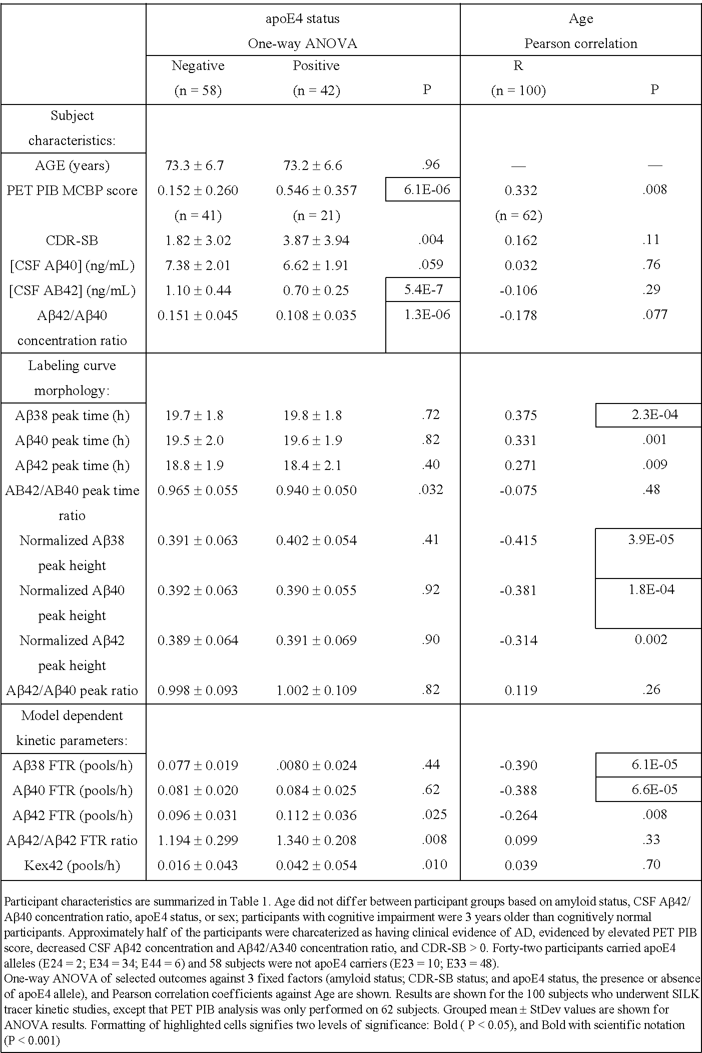

### Example 4

### CNS Aβ42 Kinetics are Altered with Amyloidosis

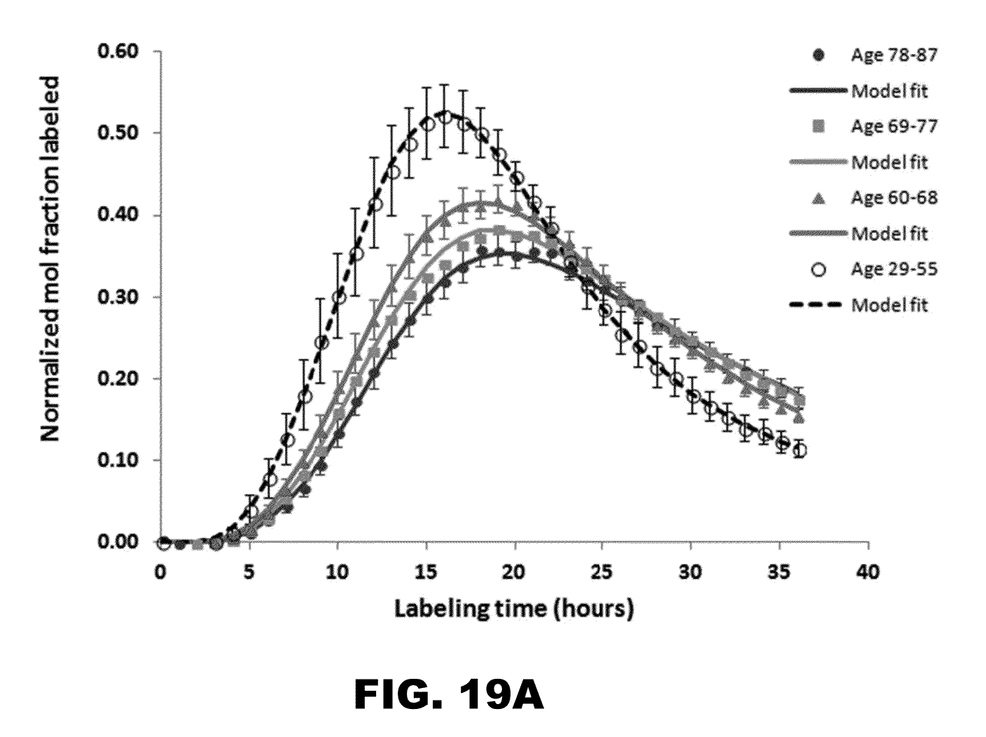

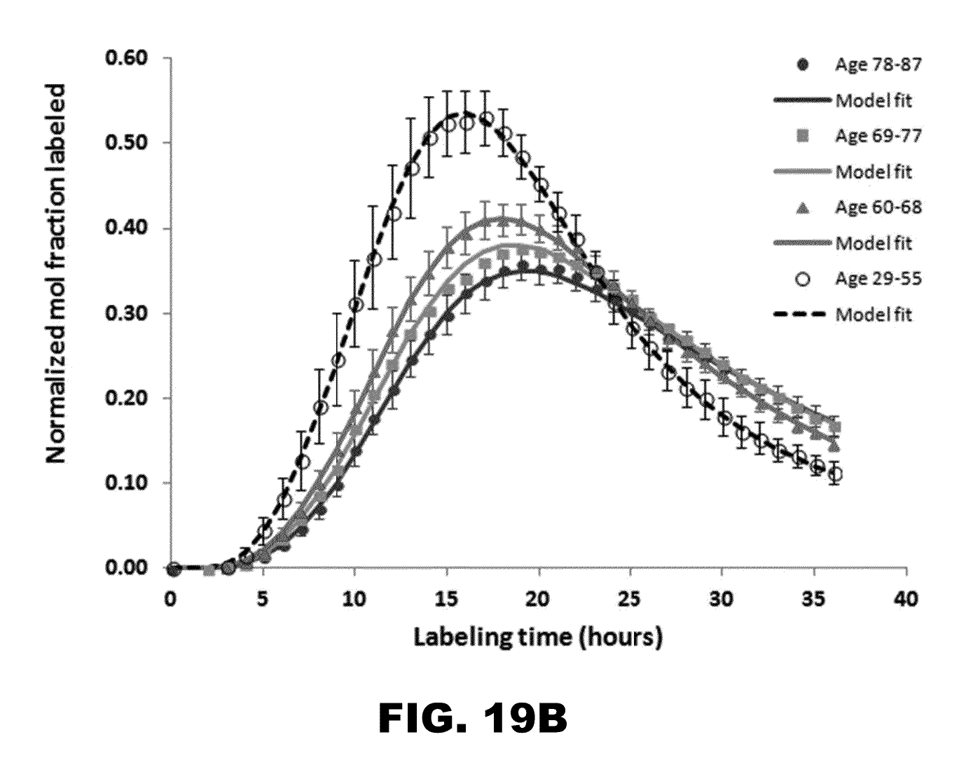

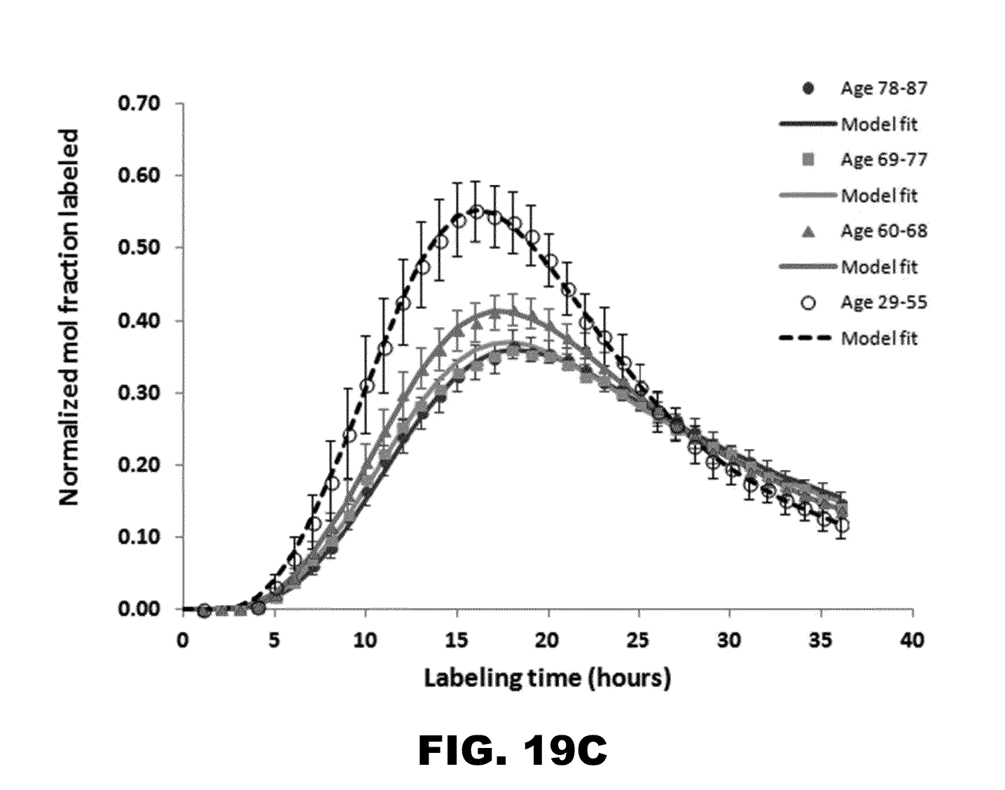

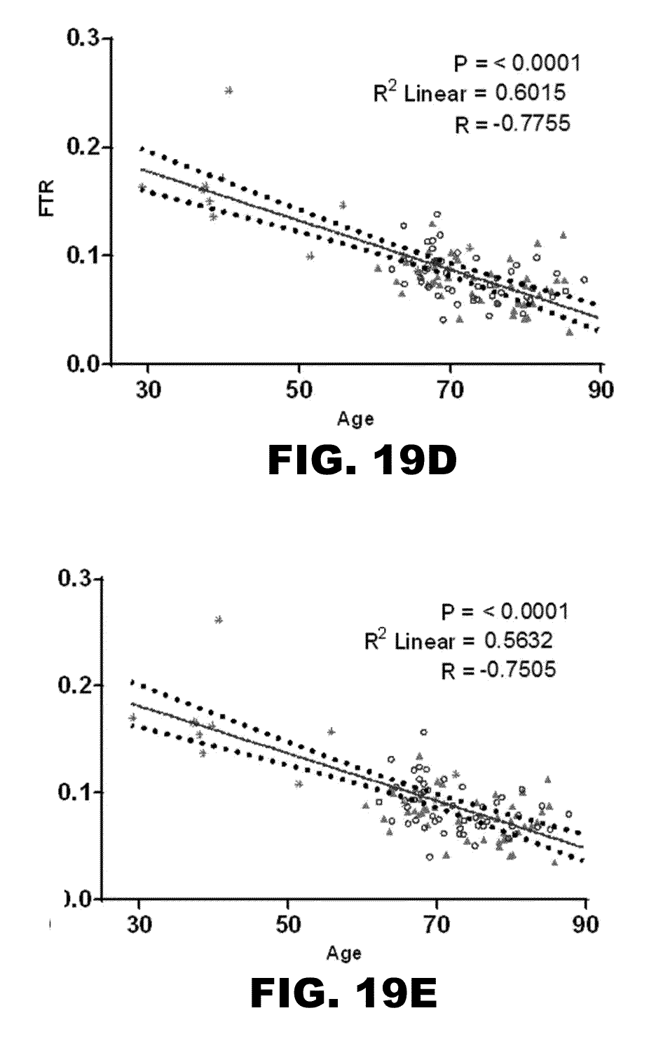

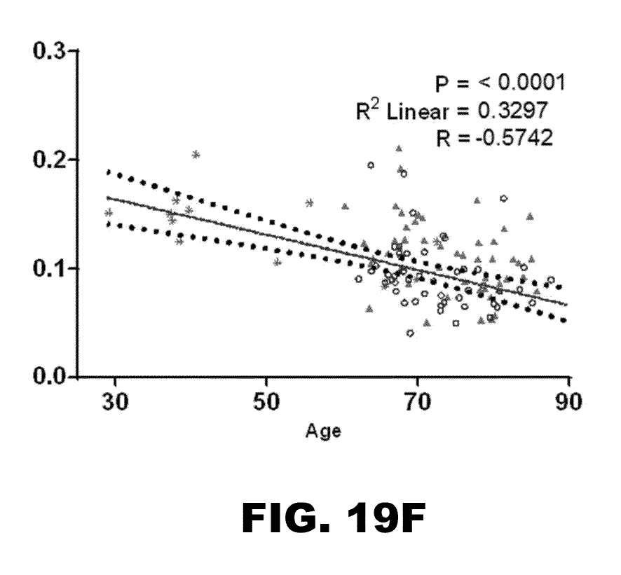

In order to understand how age affects Aβ kinetics, Aβ isoform kinetics were compared over a broad age range (30's to 90's). Aβ38, Aβ40 and Aβ42 turnover rates were negatively correlated with participant age as demonstrated by the SILK tracer kinetic time course for each Aβ isoform (FIG. 19). There were significant negative correlations between age and the FTRs of Aβ38 (r=−0.77), Aβ40 (r=−0.75) and Aβ42 (r=−0.57) (FIG. 19). The turnover of Aβ slowed by approximately two-fold from 30 to 70 years of age with high consistency. The correlation of turnover with age was lower for Aβ42 than Aβ38 and Aβ40 due to the presence of plaques. This age-associated slowing of Aβ peptide turnover affects all Aβ isoforms in a parallel fashion, and is independent of the amyloid-associated alterations.

### Example 5

### Interaction Between Amyloidosis and Cognitive Impairment for Aβ Isoform Kinetics

Although the mean FTR of Aβ38 (0.078±0.021) and Aβ40 (0.082±0.022) was not significantly different by univariate analysis of amyloid status (Table 1), significant differences were present with an interaction of amyloid status and cognitive impairment (Amyloid*CDR). FTR was significantly faster in Aβ42 (57% faster), and to a lesser extent for Aβ40 (17% faster) and Aβ38 (22% faster) in the cognitively normal, amyloid positive group compared to the cognitively normal, amyloid negative group (Table 2). There was no interaction between cognitive status and amyloid status for Aβ42 exchange (kₑₓ42, Table 2).

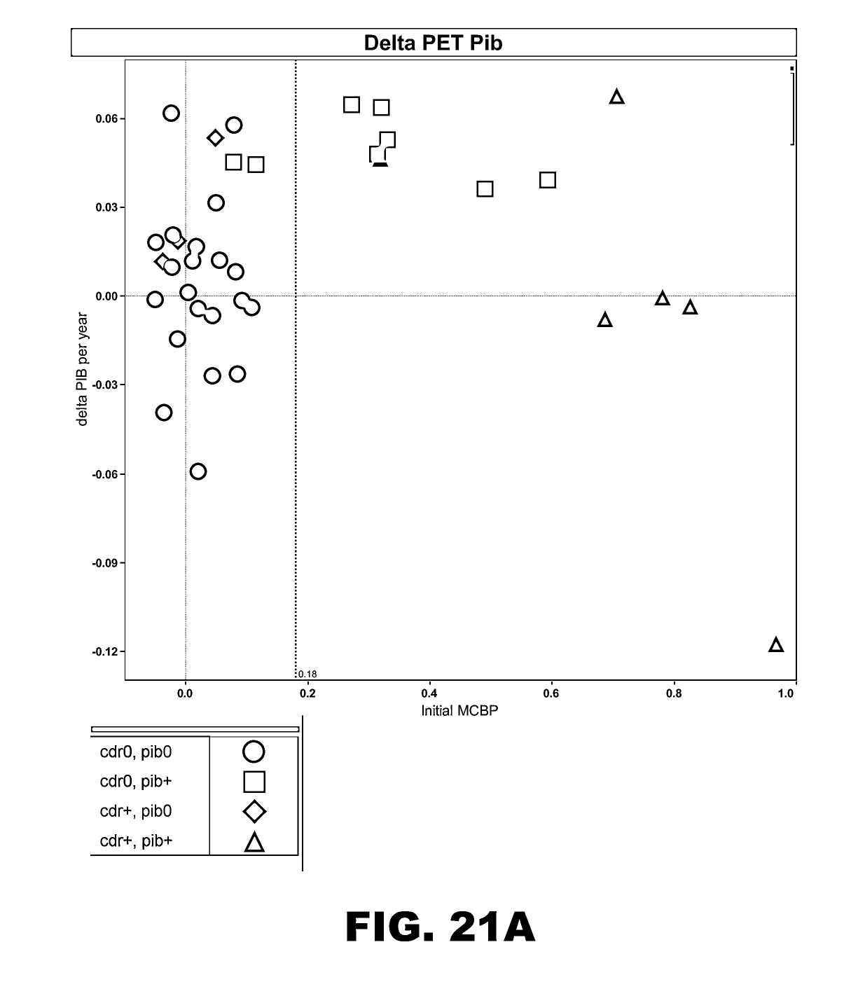

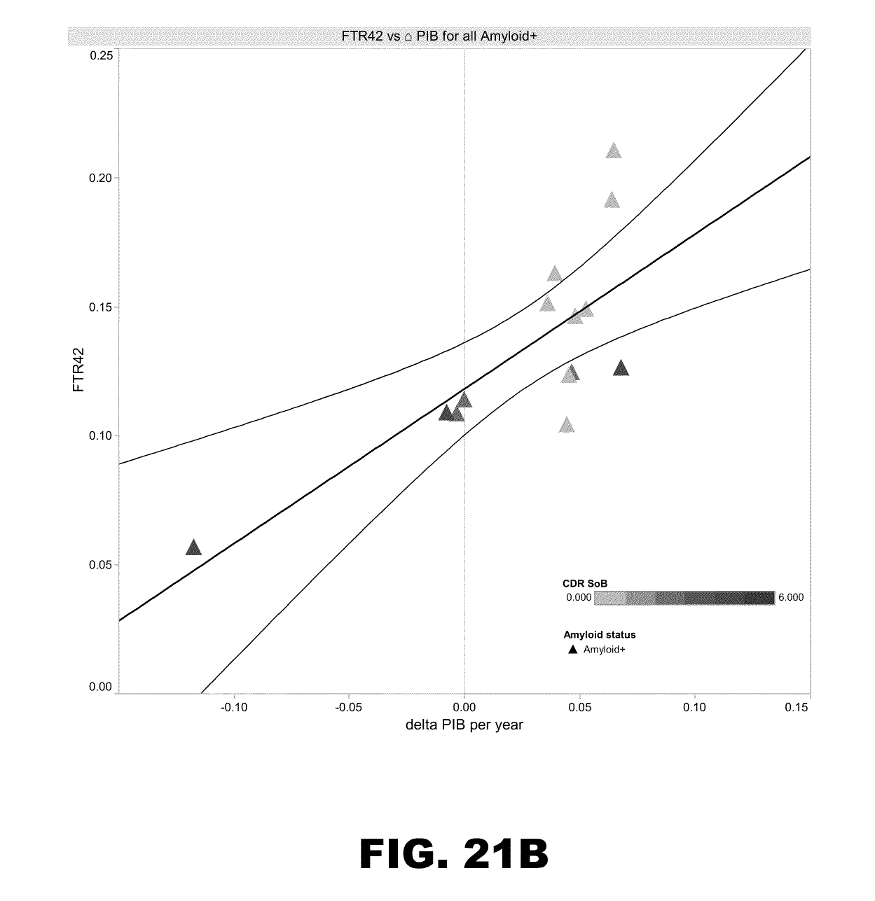

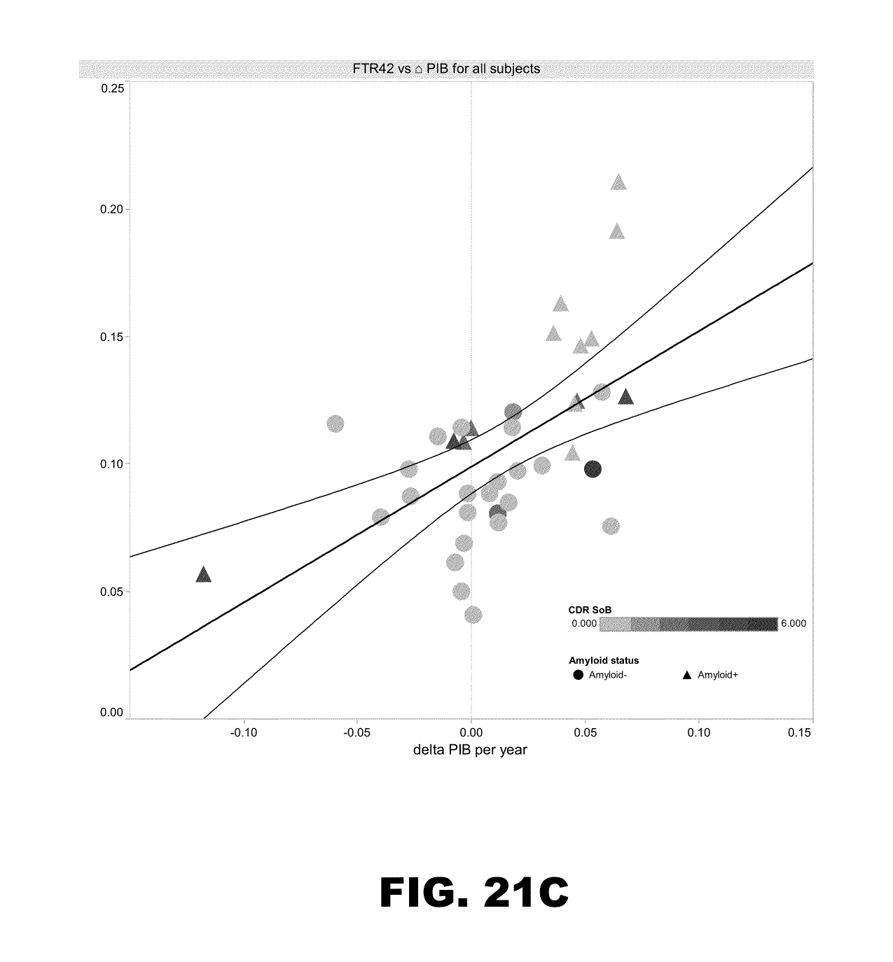

In order to evaluate this interaction, the active fibrillar amyloid deposition were compared by calculating the change in PIB over time and compared to the Aβ42 FTR. In participants who received initial and follow-up PIB scans, the change in PIB per year was greater in the cognitively normal, PIB(+) group than the cognitively normal, PIB(−) group (0.049±0.011 vs. 0.003±0.025). Consistent with the FTR Aβ42, there was a decrease in fibrillar amyloid deposition in the cognitively affected, PIB(+) group (−0.002±0.064). Substantial increases in the rate of PIB increase were present in all cognitively normal PIB(+), but decreased after participants were cognitively affected (FIG. 21A). There was a positive correlation in PIB+ participants of R=0.75, p=0.002 (FIG. 21B) and in both PIB+ and PIB− participants, R=0.56, p=0.0002 (FIG. 21C).

TABLE 2
Multivariate ANOVA analysis of amyloid status, age,
and cognitive impairment on Aβ SILK parameters

| Param- | | Overall | |
|---|---|---|---|
| eters | Effect | p-value | Mean(95% confidence limits) |
| Aβ42 | Amyloid | 0.02 | Amyloid+ 0.124 (0.112-0.135) |
| FTR | | | Amyloid− 0.101 (0.086-0.115) |
| | CDR*Am- | 0.002 | CDR = 0 Amyloid− 0.091(0.070-0.111) |
| | yloid | | CDR = 0 Amyloid+ 0.143(0.124-0.163) |
| | | | CDR > 0 Amyloid− 0.110(0.093-0.128) |
| | | | CDR > 0 Amyloid+ 0.104(0.093-0.115) |
| | age | 0.21 | — |
| Aβ40 | Amyloid | 0.62 | — |
| FTR | CDR*Am- | 0.007 | CDR = 0 Amyloid− 0.082(0.068-0.096) |
| | yloid | | CDR = 0 Amyloid+ 0.096(0.083-0.109) |
| | | | CDR > 0 Amyloid− 0.094(0.083-0.106) |
| | | | CDR > 0 Amyloid+ 0.074(0.067-0.081) |
| | Age | 0.012 | — |
| Aβ38 | Amyloid | 0.97 | — |
| FTR | CDR*Am- | 0.007 | CDR = 0 Amyloid− 0.075(0.062-0.088) |
| | yloid | | CDR = 0 Amyloid+ 0.092(0.079-0.104) |
| | | | CDR > 0 Amyloid− 0.088(0.077-0.099) |
| | | | CDR > 0 Amyloid+ 0.071(0.064-0.078) |
| | age | 0.01 | — |
| Aβ42/ | Amyloid | <.0001 | Amyloid+ 1.448(1.363-1.533) |
| Aβ40 | | | Amyloid− 1.145(1.036-1.254) |
| FTR | CDR*Am- | 0.47 | — |
| ratio | yloid | | |
| | age | 0.16 | — |
| kₑₓ₄₂ | Amyloid | 0.001 | Amyloid+ 0.059(0.042-0.076) |
| | | | Amyloid− 0.006(−0.016-0.029) |
| | CDR*Am- | 0.13 | — |
| | yloid | | |
| | age | 0.48 | — |

### Example 6

### ApoE4 Effects

The effect of Apolipoprotein E4 (ApoE4) allele for Aβ kinetic alterations was evaluated. The majority of participants with ApoE4 had clear evidence of amyloidosis: of the 42 participants with one or more ApoE4 alleles, 34 (81%) were characterized as amyloid positive; 33 (79%) had CSF Aβ42/Aβ40 concentration ratio<=0.12; and 30 (71%) had cognitive impairment (CDR-SB>0). PET PIB score was available in 21 ApoE4 carriers; 17 (81%) of these had PET PIB MCP>0.18. Thus, when one-way ANOVA was performed using ApoE4 status, the outcomes were generally consistent with the presence of amyloid plaques in participants carrying the ApoE4 allele (Table 1). No significant effects of ApoE4 on the exchange of Aβ42 or Aβ kinetic rates were observed by multivariate analysis when amyloid status was included as a factor in the analysis. Thus, given the high association between ApoE4 and the presence of amyloid plaques, ApoE4 effects independent of amyloid status in this study could not be determined.

### Example 7

### Conclusions for Examples 3-7

Examples 3-7 provide the first comprehensive analysis of Aβ isoform kinetics in humans by age and amyloidosis. These findings are the first to link Aβ with age, which is the single largest risk factor for AD.2, 17, 18 Aβ turnover rate was highly correlated with age and is an excellent biomarker for chronological age (Pearson correlation of 0.77, FIG. 19).¹⁹ The remarkable two-fold slowing of Aβ turnover rate over four decades may account for the increasing liability of amyloidosis associated with aging. Amyloidosis then greatly increases the risk of cognitive decline and AD.²⁰⁻²³ For example, as Aβ clearance rate from the CNS decreases, Aβ may be more liable to aggregation or modification. This finding may also explain why early onset (e.g. 30's to 50's) dominantly inherited AD also has a clear age-dependent onset.⁵ This study found clear and significant alterations in Aβ42 kinetics in the presence of amyloidosis. The soluble Aβ42 irreversible loss (FTR) is faster in the presence of amyloidosis, while an increased exchange process increases the time that Aβ42 is present in the CNS. These findings are similar to those seen in amyloid positive autosomal dominant AD,¹⁴ suggesting that once amyloid deposition occurs, ADAD and sporadic AD have a common pathophysiology in amyloidosis and altered soluble Aβ42 kinetic rates.

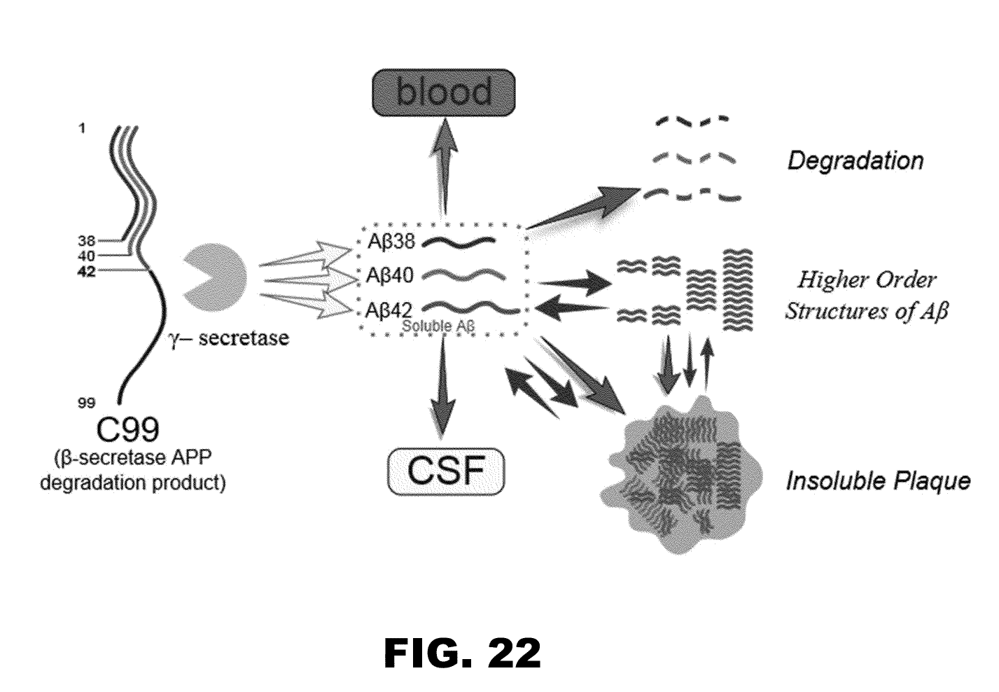

Two aspects of Aβ42 kinetics are altered in amyloidosis. First, there is a ten-fold increase in the exchange of newly synthesized soluble Aβ42 with one or more pools, which may represent higher-order aggregates such as oligomers or the surface of plaques. Second, there is increased irreversible loss of soluble Aβ42, perhaps due to irreversible aggregation in higher order Aβ structures such as oligomers or amyloid plaques. Without wishing to be bound by theory, applicants have proposed a biological hypothesis to account for the current understanding of CNS Aβ biology in amyloidosis which fits with the Aβ kinetic measures disclosed above (FIG. 22). Future testing of this model can be addressed with animal model studies, longitudinal clinical observations, or interventional studies. For example, a drug which blocks new oligomer, protofibril or plaque formation would be expected to normalize the exchange and irreversible loss.

An interaction in the turnover rate of Aβ42 with amyloidosis and clinical symptoms was noted. The change in insoluble amyloid by PET PIB also demonstrates a similar interaction (FIG. 21). Taken together, these findings suggest that Aβ42 FTR is a measure of irreversible loss due to plaque deposition. Lesser effects and interactions were noted for Aβ40 and Aβ38 FTR, suggesting these isoforms are also affected to a lesser degree.

ApoE allele genotype was evaluated with respect to Aβ kinetics, however, ApoE4 was highly associated with amyloidosis so that independent comparisons could not be made. Studies of Aβ kinetics by ApoE genotype in younger participants are likely to inform about the potential impact of ApoE on Aβ metabolism before amyloidosis occurs and potentially an Aβ mechanism for ApoE4's increased risk of AD.

The Aβ kinetic model can be used to estimate how long Aβ42 would need to accumulate to reach amounts of amyloidosis typical of AD. The model provides a clear and highly identifiable estimate of the increase in Aβ42 turnover in late onset AD. Comparing the clinically normal amyloid positive group (FTR Aβ42=0.143 pool/h) to the clinically normal amyloid negative group (FTR Aβ42=0.091 pool/h) we propose that this difference is due to active deposition into amyloid plaques (deposition of Aβ42 rate constant=0.143-0.091=0.052 pool/h). Using literature values for the pool size of soluble Aβ42 (0.2-1 ng per gram of brain tissue), for an AD brain mass of 1100 g²⁴, this is a pool size of 220-1100 ng). The deposition rate constant multiplied by the pool size yields an approximate estimate of the rate of deposition of Aβ42 into plaques. Using a value from the median of this range (600 ng) yields a rate of deposition of Aβ42 into plaques of 600 ng*0.052 pools/hr=31.2 ng/h, or 273 μg/year. The amount of insoluble Aβ42 in AD brains is in the range of 0.1-20 μg per gram of brain tissue, or 0.5-60 mg per brain²⁵⁻²⁹. Using an intermediate value of 11 mg per brain, the simple calculation estimates that plaques build up over about 40 years. Recent results³⁰ of PET PIB longitudinal accumulation rates suggest that approximately 40 years of amyloid accumulation occur in AD, while other estimates are 15-20 years.^{21,31} This simple model does not account for potentially exponential growth in the early to middle phase of plaque growth nor does it predict a plateau in plaque loads as seen in previous studies.^{23,31} However, it does suggest that the value for FTR Aβ42 in AD is of the correct order of magnitude for the estimated 15-20 years of amyloid growth reported in observational studies.^{23,31,32}

These findings provide a first link between aging, Aβ kinetics, and amyloidosis which will assist in the design of observational and interventional studies in AD. Future studies into the causes of slowed Aβ turnover rates associated with aging may lead to prevention strategies for amyloidosis. The concept that protein kinetics reveals changes in the physiology of aging and amyloid disorders may be applied to other disease states such as sarcopenia, osteoporosis, heart failure, and cancer. Further, they provide a framework for studying protein kinetics and physiological changes in aging and disease states.

### Methods for Examples

These human studies took place at the Washington University School of Medicine in St. Louis and were approved by the Washington University Human Studies Committee and the General Clinical Research Center (GCRC) Advisory Committee. All participants completed informed written consent. One hundred and one participants were enrolled for these studies comprising 57 men (aged from 60.4 to 87.7) and 44 women (aged 63.8 to 85.2). Deposition of amyloid plaques was quantified in 62 subjects based on the mean cortical binding potential (MCBP) score of [¹¹C]PIB-PET.¹⁵ PET PIB scans were performed within 3 years before or after the SILK tracer study date. Cognitive status using the Clinical Dementia Rating sum of boxes score (CDR-SB16) and ApoE genotyping³³ was assessed in all subjects. Amyloid status was assigned based on PIB score, if available (amyloid positive if MCBP PIB score>0.18), or based on CSF Aβ42/40 concentration ratio if PIB score was not available (amyloid positive if Aβ42/40 concentration ratio<0.12). Participant characteristics are presented in Table 1.

Aβ SILK data of 12 younger amyloid negative subjects that were previously published14 were included for assessment of age effects. These subjects were non-carriers of presenilin mutations; 5 male/7 female; age 48.0±14.6 (range 29.2-72.6 years); MCBP PIB score 0.026±0.045 (range −0.026 to 0.120); all CDR=0; apoE genotypes: E23 (n=2), E33 (n=6), E34 (n=4).

Description of Tracer Protocol & Sample Collection:

The procedure for stable isotope amino acid tracer administration and sample collection was previously described.¹² Briefly, intravenous and intrathecal lumbar catheters were placed between 7:00 AM and 9:00 AM, and the collection of samples was started between 8:00 AM and 10:00 AM. After initial CSF and plasma baseline samples were collected, each participant was infused with a bolus of 3 mg/kg L-[U-¹³C₆] leucine for 10 minutes, followed by a constant infusion (2 mg/kg/h) for the remainder of the first 9 hours. Blood samples (12 mL) were obtained hourly for the first 16 h and every other hour thereafter. CSF samples (6 mL) were obtained hourly throughout each study. Aliquots of plasma and CSF were frozen at −80° C. immediately in 1 mL pdypropylene tubes after being collected for subsequent determination of plasma leucine and CSF Aβ isoform peptide enrichment. The sample collection for one subject was truncated at 4 hours; this subject was excluded from the analysis.

Aβ SILK:

All samples were processed and measured in a blinded fashion with data results and individual analysis completed before unblinding to participant's disease state. The procedure for sample preparation, data acquisition and processing were previously described.³⁴ Briefly, 1 mL of CSF from each hour of collection and media standards were thawed. Aβ was purified and processed for mass spectrometry by immunoprecipitation with a mid-domain binding antibody HJ5.1 (Aβ amino acids 13 to 28). The immunopurification mixture was comprised of 800 μL CSF, 20 μL of a solution containing uniformly ¹⁵N-labeled Aβ40 (10 ng), Aβ42 (1 ng), and Aβ38 (1.5 ng) as internal standard; 12.5 μL 100× protease inhibitor, 110 μL 5M guanidine hydrochloride in 50 mM Tris-HCl (pH 8.0), and 30 μL antibody-bead slurry (50% PBS). The purified Aβ was then digested with Lys-N(Metalloendopeptidase) and isotopic enrichment of Aβ c-terminal isoform specific peptides (Aβ29-38, Aβ29-40, and Aβ29-42) were measured using a nano-liquid chromatography (NanoLC-2D-Ultra system (Eksigent Technologies, CA USA)) coupled to a TSQ Vantage triple quadrupole mass spectrometer (ThermoScientific, San Jose USA) that was equipped with a column-heating nanospray source (Phoenix S&T, Chester Pa. USA). Xcalibur V2.1 was used to collect and quantify the mass spectrometry data for SILK. Aβ SILK tracer kinetics for 24 participants were previously reported13 using a different analytical method of immunoprecipitation with Aβ42 and Aβ40 specific antibodies and measuring only the Aβ mid-domain peptide. Samples for 100 subjects (including the prior 24 subjects) were analyzed utilizing the more specific and sensitive method34 in this study.

For determination of plasma ¹³C₆-leucine enrichment, amino acids were recovered from plasma using cation exchange chromatography, converted to N-heptafluorobutyryl n-propyl ester derivatives, and ¹³C₆-leucine enrichment (tracer:tracee ratio) was quantified by selected ion monitoring (m/z 349 and 355) using gas chromatography-negative chemical ionization-mass spectrometry (Agilent 6890N Gas Chromatograph and Agilent 5973N Mass Selective Detector (GC-MS); Agilent, Palo Alto, Calif.) as described.35

CSF Concentrations:

Absolute quantification of Aβ38, Aβ40, and Aβ42 isoform amounts in CSF was measured by isotope dilution mass spectrometry. One mL aliquots of CSF (t=0) from each of 101 participants were randomly split into 2 groups: 50 and 51 respectively and processed the same way as SILK protocol described above and analyzed as 2 consecutive assays. An artificial CSF was made comprised of serial dilutions of synthetic standards of Aβ38, Aβ40 and Aβ42 (rPeptide, Bogart, Ga.) into PBS with protease-free BSA added as a carrier protein. The Aβ standard was diluted two-fold in serial fashion with ranges starting from 7.5 ng to 0.47 ng for Aβ38, 50 ng to 3.12 ng for Aβ40 and 5 ng to 0.31 ng for Aβ42. The generated calibration curve was spiked with 20 μL of a solution containing uniformly ¹⁵N-labeled Aβ38, Aβ40, and Aβ42 (rPeptide, Bogart, Ga.) as internal standard, consisting of 1.5 ng of Aβ38, 10 ng of Aβ40 and 1 ng of Aβ42. The resulting standard curves for the Aβ isoforms were used to calculate the concentration of Aβ isoforms in CSF. LC-MS/MS measurements were performed on Waters Xevo TQ-S triple quadrupole mass spectrometer (Waters Inc., Milford, Mass.) coupled to Waters nano-ACQUITY UPLC and equipped with Waters BEH130 nanoAcquity UPLC column (C18 particle, 1.7 μm, 100 μm×100 mm) and Waters nano-ESI ionization source. Data was acquired and quantified using Waters MassLynx 4.1 software suite.

Kinetics Analysis:

The time and magnitude of the peak maximum was determined by fitting a 2nd order curve to enrichment vs. time over an 11-h interval centered at the approximate peak maximum. The compartmental model previously developed and applied to Autosomal Dominant AD participants was used to determine model-dependent parameters of Aβ turnover kinetics (FIG. 18).¹⁴ Modeling was performed using the Population Kinetics (PopKinetics, version 1.0.1) companion application to the SAAM II modeling program (version 1.2.1, SAAM Institute, University of Washington, Seattle). PopKinetics performed an iterative two-stage approach to optimize kinetic parameters to individual participants as well as the population as a whole by including a term that represents the population mean and standard deviation for each adjustable parameter such that variability of the kinetic parameters across the population is minimized. Parameters of the compartmental model were adjusted to optimally fit the shape and magnitude of the enrichment time course, including the FTR for irreversible loss (affects the peak time, peak magnitude, and steepness of the rise and fall from the peak), an exchange process for Aβ42 (affects the shape of the back end of the curve, primarily when amyloidosis is evident), and a delay time (affects the time at which labeled peptides are detected at the lumbar sampling site). The model consists of a plasma leucine pool, a subsystem for production of Aβ through APP and C99, turnover of Aβ, and transport of Aβ to CSF. Fluid transport through the CSF is depicted as a system of 3 compartments, which together with the APP and C99 compartment represent a total of 5 compartments that comprise a time delay that is common to all Aβ isoforms, which can be resolved from the turnover of Aβ.14 Aβ42 exchanges with another compartment, particularly when amyloid plaques are present, which alters the shape of the back end of the tracer time course and causes isotopic dilution of the peak enrichment for Aβ42. An exchange process for Aβ38 and Aβ40 was initially included in the model,¹⁴ but PopKinetics optimized this process to zero for all participants regardless of the presence of amyloid plaques so this process was removed from the model. Tracer to tracee ratios (TTR) obtained from the mass spectrometric analysis for plasma leucine, Aβ38, Aβ40 and Aβ42 were converted to mole fraction labeled for modeling analysis, as this measure of isotopic enrichment has been shown to be most appropriate for compartmental modeling of stable isotope tracer data.³⁶

The principal parameters obtained from the kinetic analysis include: the fractional turnover rate (FTR) for the irreversible loss of each peptide, which is the sum of losses to CSF (k_{CSF}) and other loss pathways (v38, v40, or v42); kₑₓ42; k_{delay} (turnover rate of each CSF delay compartment, which is also the turnover rate of APP [k_{C99}] and the sum of all losses from C99); k_{APP} (production rate of APP); and the individual rate constants for Aβ peptide production from C99 (k_{Aβxx}). k_{CSF40} was assumed to be 50% of FTR_{Aβ40}, with the same value applied to k_{CSF38} and k_{CSF42} since bulk fluid transport from the brain into CSF is expected to be equivalent for all peptides, and v_{C99} was assumed to be 50% of the total turnover rate of C99.14 Major conclusions of the study concerning the impact of amyloidosis and age on Aβ kinetics were not affected by these assumptions. PopKinetics optimized 11 adjustable parameters against the measured enrichments and CSF concentrations for Aβ38, Aβ40 & Aβ42 for each participant: the FTR for each peptide; kₑₓ42; k_{delay}; k_{APP}; rate constants for production of Aβ38 and Aβ42 (k_{Aβ38} and k_{Aβ42}; k_{Aβ40} is determined based on these values and other constraints); and a scaling factor for each Aβ peptide that accounts for any isotopic dilution between plasma leucine and the precursor pool for APP synthesis and sources of analytical error that systematically affect the accuracy of isotopic enrichment measurements for a given set of samples.

Statistical Analysis:

The amyloid status of 62 participants was defined by their PIB scores. Participants were classified as positive or negative using the published threshold (i.e., a PIB score of less or equal than 0.18 is defined as Amyloid negative while a PIB score of greater than 0.18 is defined as Amyloid positive. In the absence of PIB score, the amyloid status of the remaining 28 articipants was defined by the CSF Aβ42/40 ratio, using a cut-off of 0.12 based on a ROC curve analysis. The cut-off value was achieved by maximizing the sum of sensitivity and specificity. All analyses were conducted in SAS, version 9.3 (SAS Institute). Statistical significance was defined by p<0.05.

Each kinetic parameter was further analyzed using the analysis of covariance (ANCOVA) models in which age was treated as a continuous covariate (centered at the mean) and amyloid status, clinical status (cognitively impaired or not), and/or APOE4 were treated as classification predictors. All possible interactions across these variables were included in the model first. When the highest order of interaction was not significant, a reduced model was then fitted after removing the highest order of interaction. When there were no interactions between age and any other classification variables, the age-adjusted main as well as interactive effects of amyloid status, clinical status, and APOE4 were then reported. Further analyses were also conducted to examine the effects of other covariates such as gender. All these analyses were implemented in PROC GLM/SAS.

### REFERENCES

1. Thies W, Bleiler L, Alzheimer's A. 2013 Alzheimer's disease facts and figures Alzheimer's Association. Alzheimers & Dementia 2013; 9:208-245.
2. Jorm A F, Jolley D. The incidence of dementia-A meta-analysis. Neurology 1998; 51:728-733.
3. Qiu C, Kivipelto M, von Strauss E. Epidemiology of Alzheimer's disease: occurrence, determinants, and strategies toward intervention. Dialogues in clinical neuroscience 2009; 11:111-128.
4. Roses A D, Saunders A M. APOE is a major susceptibility gene for Alzheimer's disease. Current opinion in biotechnology 1994; 5:663-667.
5. Ryman D C, Acosta-Baena N, Aisen P S, et al. Symptom onset in autosomal dominant Alzheimer disease: A systematic review and meta-analysis. Neurology 2014; 83:253-260.
6. Goate A, Chartierharlin M C, Mullan M, et al. SEGREGATION OF A MISSENSE MUTATION IN THE AMYLOID PRECURSOR PROTEIN GENE WITH FAMILIAL ALZHEIMERS-DISEASE. Nature 1991; 349:704-706.
7. Stgeorgehyslop P, Haines J, Rogaev E, et al. GENETIC-EVIDENCE FOR A NOVEL FAMILIAL ALZHEIMERS-DISEASE LOCUS ON CHROMOSOME-14. Nature Genetics 1992; 2:330-334.
8. Levylahad E, Wasco W, Poorkaj P, et al. CANDIDATE GENE FOR THE CHROMOSOME-1 FAMILIAL ALZHEIMERS-DISEASE LOCUS. Science 1995; 269:973977.
9. Sherrington R, Rogaev E I, Liang Y, et al. CLONING OF A GENE BEARING MISSENSE MUTATIONS IN EARLY-ONSET FAMILIAL ALZHEIMERS-DISEASE. Nature 1995; 375:754-760.
10. Jonsson T, Atwal J K, Steinberg S, et al. A mutation in APP protects against Alzheimer's disease and age-related cognitive decline. Nature 2012; 488:96-99.
11. Selkoe D J. PHYSIOLOGICAL PRODUCTION OF THE BETA-AMYLOID PROTEIN AND THE MECHANISM OF ALZHEIMERS-DISEASE. Trends in Neurosciences 1993; 16:403-409.
12. Bateman R J, Munsell L Y, Morris J C, Swarm R, Yarasheski K E, Holtzman D M. Human amyloid-beta synthesis and clearance rates as measured in cerebrospinal fluid in vivo. Nature medicine 2006; 12:856-861.
13. Mawuenyega K G, Sigurdson W, Ovod V, et al. Decreased Clearance of CNS beta-Amyloid in Alzheimer's Disease. Science 2010; 330:1774-1774.
14. Potter R, Patterson B W, Elbert D L, et al. Increased in Vivo Amyloid-beta 42 Production, Exchange, and Loss in Presenilin Mutation Carriers. Science Translational Medicine 2013; 5.
15. Mintun M A, LaRossa G N, Sheline Y I, et al. (11) PIB in a nondemented population—Potential antecedent marker of Alzheimer disease. Neurology 2006; 67:446-452.
16. Morris J C. THE CLINICAL DEMENTIA RATING (CDR)—CURRENT VERSION AND SCORING RULES. Neurology 1993; 43:2412-2414.
17. Gao S, Hendrie H C, Hall K S. The relationships between age, sex, and the incidence of dementia and Alzheimer disease—A meta-analysis. Archives of General Psychiatry 1998; 55:809815.
18. Barnes D E, Yaffe K. The projected effect of risk factor reduction on Alzheimer's disease prevalence. Lancet Neurology 2011; 10:819-828.
19. von Zglinicki T, Martin-Ruiz C M. Telomeres as biomarkers for ageing and age-related diseases. Current Molecular Medicine 2005; 5:197-203.
20. Doraiswamy P M, Sperling R A, Coleman R E, et al. Amyloid-beta assessed by florbetapir F 18 PET and 18-month cognitive decline A multicenter study. Neurology 2012; 79:1636-1644.
21. Ewers M, Insel P, Jagust W J, et al. CSF Biomarker and PIB-PET-Derived Beta-Amyloid Signature Predicts Metabolic, Gray Matter, and Cognitive Changes in Nondemented Subjects. Cerebral Cortex 2012; 22:1993-2004.
22. Lim Y Y, Maruff P, Pietrzak R H, et al. Effect of amyloid on memory and non-memory decline from preclinical to clinical Alzheimer's disease. Brain 2014; 137:221-231.
23. Bateman R J, Xiong C, Benzinger T L S, et al. Clinical and Biomarker Changes in Dominantly Inherited Alzheimer's Disease. New England Journal of Medicine 2012; 367:795804.
24. Arnold S E, Hyman B T, Flory J, Damasio A R, Van Hoesen G W. The Topographical and Neuroanatomical Distribution of Neurofibrillary Tangles and Neuritic Plaques in the Cerebral Cortex of Patients with Alzheimer's Disease. Cerebral Cortex 1991; 1:103-116.
25. Roher A E, Esh C L, Kokjohn T A, et al. Amyloid beta peptides in human plasma and tissues and their significance for Alzheimer's disease. Alzheimers & Dementia 2009; 5:18-29.
26. Hellstrom-Lindahl E, Viitanen M, Marutle A. Comparison of A beta levels in the brain of familial and sporadic Alzheimer's disease. Neurochemistry International 2009; 55:243-252.
27. Gravina S A, Ho L B, Eckman C B, et al. AMYLOID-BETA PROTEIN (A-BETA) IN ALZHEIMERS-DISEASE BRAIN—BIOCHEMICAL AND IMMUNOCYTOCHEMICAL ANALYSIS WITH ANTIBODIES SPECIFIC FOR FORMS ENDING AT A-BETA-40 OR A-BETA-42(43). Journal of Biological Chemistry 1995; 270:7013-7016.
28. Naslund J, Haroutunian V, Mohs R, et al. Correlation between elevated levels of amyloid beta-peptide in the brain and cognitive decline. Jama-Journal of the American Medical Association 2000; 283:1571-1577.
29. Lewis H, Beher D, Cookson N, et al. Quantification of Alzheimer pathology in ageing and dementia: age-related accumulation of amyloid-beta(42) peptide in vascular dementia. Neuropathology and Applied Neurobiology 2006; 32:103-118.
30. Masters C. How to Change and Monitor the Rates of Aβ Amyloid Accumulation and Cognitive Decline in Alzheimer's Disease. Copenhagen, Denmark: Alzheimer's Association International Conference, 2014.
31. Jack C R, Jr., Knopman D S, Jagust W J, et al. Tracking pathophysiological processes in Alzheimer's disease: an updated hypothetical model of dynamic biomarkers. Lancet Neurology 2013; 12:207-216.
32. Rowe C C, Ellis K A, Rimajova M, et al. Amyloid imaging results from the Australian Imaging, Biomarkers and Lifestyle (AIBL) study of aging. Neurobiology of Aging 2010; 31:1275-1283.
33. Head D, Bugg J M, Goate A M, et al. Exercise Engagement as a Moderator of the Effects of APOE Genotype on Amyloid Deposition. Archives of Neurology 2012; 69:636-643.
34. Mawuenyega K G, Kasten T, Sigurdson W, Bateman R J. Amyloid-beta isoform metabolism quantitation by stable isotope-labeled kinetics. Analytical Biochemistry 2013; 440:56-62.
35. Reeds D N, Cade W T, Patterson B W, Powderly W G, Klein S, Yarasheski K E. Whole-body proteolysis rate is elevated in HIV-associated insulin resistance. Diabetes 2006; 55:28492855.
36. Ramakrishnan R. Studying apolipoprotein turnover with stable isotope tracers: correct analysis is by modeling enrichments. Journal of Lipid Research 2006; 47:2738-2753.

What is claimed is:

1. A method for detecting Aβ amyloidosis in a subject, the method comprising measuring the in vivo relative labeling of at least two Aβ variants in a blood sample from the subject, and calculating a ratio of relative labeling of the first Aβ variant to the relative labeling of the second Aβ variant, wherein a ratio other than 1 indicates the presence of Aβ amyloidosis.

2. The method of claim 1, wherein the in vivo relative labeling of the at least two Aβ variants is measured by:
a. administering a labeled amino acid to the subject;
b. obtaining a biological sample from the subject, the biological sample comprising an Aβ variant fraction labeled with the moiety and an Aβ variant fraction not labeled with the moiety; and
c. detecting the amount of labeled Aβ variant and the amount of unlabeled Aβ variant, wherein the ratio of labeled Aβ variant to unlabeled Aβ variant represents the relative labeling of said Aβ variant in the subject.

3. The method of claim 2, wherein the at least two Aβ variants are selected from a group consisting of Aβtotal, Aβ38, Aβ40 and Aβ42.

4. The method of claim 3, wherein the relative labeling of Aβ variants are measured at about 1 minute to about 4 hours after administering a labeled moiety to the subject.

5. The method of claim 4, wherein a ratio of relative labeling of Aβ42 to an Aβ variant, measured at about 1 minute to about 4 hours, of more than 1 at indicates the presence of Aβ amyloidosis.

6. The method of claim 5, wherein the relative labeling of Aβ variants are measured at about 3 hours.

7. The method of claim 3, wherein the relative labeling of Aβ variants are measured at about 8 to about 24 hours after administering a labeled moiety to the subject.

8. The method of claim 7, wherein a ratio of relative labeling of Aβ42 to an Aβ variant, measured at about 8 to about 24 hours, of less than 1 indicates the presence of Aβ amyloidosis.

9. The method of claim 8, wherein the relative labeling of Aβ variants are measured at about 24 hours.

10. The method of claim 1, wherein the labeled amino acid comprises a non-radioactive isotope selected from the group consisting of ²H, ¹³C, ¹⁵N, ¹⁷O, ¹⁸O, ³³S, ³⁴S, and ³⁶S.

11. The method of claim 2, wherein the labeled amino acid is ¹³C₆-leucine.

12. The method of claim 2, wherein the labeled moiety is administered to the subject intravenously, intra-arterially, subcutaneously, intraperitoneally, intramuscularly, or orally.

13. The method of claim 2, further comprising purifying the labeled protein fraction and the unlabeled protein fraction from the biological sample.

14. The method of claim 16, wherein the protein is separated by immunoprecipitation.

15. The method of claim 2, wherein the amount of labeled Aβ variant and the amount of unlabeled Aβ variant is detected by mass spectrometry.

16. A method for detecting Aβ amyloidosis in a subject, the method comprising measuring the in vivo relative labeling of Aβ42 and another Aβ variant in at least one biological sample obtained from the subject; and calculating a ratio of the Aβ42 Fractional Turnover Rate (FTR) to the other Aβ variant FTR, wherein FTR is a rate of irreversible loss of an Aβ variant from the central nervous system; and wherein a ratio of greater than about 1 indicates the presence of Aβ amyloidosis.

17. The method of claim 17, wherein the in vivo relative labeling of Aβ42 and the other Aβ variant is measured according to claim 2.

18. A method for detecting Aβ amyloidosis in a subject, the method comprising measuring the in vivo relative labeling of Aβ42 and another Aβ variant in at least two biological samples obtained from the subject; determining peak time for labeled Aβ42 and the other labeled Aβ variant; and calculating a ratio of the Aβ42 peak time to the peak time of the other Aβ variant; wherein a ratio of less than about 1 indicates the presence of Aβ amyloidosis.

19. The method of claim 19, wherein the in vivo relative labeling of Aβ42 and the other Aβ variant is measured according to claim 2.
